(19) United States
(12) Patent Application Publication
(10) Pub. No.: US 2023/0192718 A1
(43) Pub. Date: Jun. 22, 2023

(54) BICYCLIC ENONE CARBOXYLATES AS MODULATORS OF TRANSPORTERS AND USES THEREOF

(71) Applicant: NIROGY THERAPEUTICS, INC., Natick, MA (US)

(72) Inventor: Vincent Sandanayaka, Northborough, MA (US)

(21) Appl. No.: 18/172,742

(22) Filed: Feb. 22, 2023

Related U.S. Application Data
(63) Continuation of application No. 16/980,588, filed on Sep. 14, 2020, now abandoned.
(60) Provisional application No. 62/650,592, filed on Mar. 30, 2018.

(51) Int. Cl. C07D 495/04; C07D 513/04; A61P 35/00

(52) U.S. Cl. CPC C07D 495/04; C07D 513/04; A61P 35/00

(57) ABSTRACT

The invention generally relates to the field of monocarboxylate transporter inhibitors, and more particularly to new bicyclic enone carboxylate enone compounds, the synthesis and use of these compounds and their pharmaceutical compositions, e.g., in the treatment, modulation and/or prevention of physiological conditions associated with monocarboxylate transporter activity such as in treating cancer and other neoplastic disorders, tissue and organ transplant rejection.

### CROSS-REFERENCE TO RELATED APPLICATIONS

This application claims priority from U.S. provisional application 62/650,592, filed Mar. 30, 2018, which is incorporated herein by reference in its entirety.

### GOVERNMENT RIGHTS STATEMENT

This invention was made with Government support under grant 1R43CA217564-01A1 awarded by the National Institutes of Health. The Government has certain rights in the invention.

### FIELD OF THE INVENTION

The present invention relates to compounds useful as transporter modulators. The invention also provides pharmaceutically acceptable compositions comprising compounds of the present invention and methods of using said compositions in the treatment of various disorders.

### BACKGROUND

It has been well demonstrated that tumors display altered cellular metabolism, in which cancer cells exhibit high rate of glucose consumption compared to the untransformed normal cells. Tumors contain well oxygenated (aerobic), and poorly oxygenated (hypoxic) regions. Compared to normal cells, some cancer cells are heavily dependent upon either aerobic glycolysis (Warburg effect, 1956) or anerobic glycolysis (especially in hypoxic regions) for energy (ATP) production while maintaining a certain level of oxidative phosphorylation. This glycolytic switch by highly proliferating and hypoxic cancer cells provides the energy and biosynthetic needs for cancer cell survival. To maintain this metabolic phenotype, cancer cells up regulate a series of proteins, including glycolytic enzymes and pH regulators; monocarboxylate transporters (MCTs) that will facilitate the efflux of lactate co-transported with a proton. This fundamental difference between normal cells and cancer cells has not been previously applied to cancer therapy.

MCTs mediate influx and efflux of monocarboxylates such as lactate, pyruvate, ketone bodies (acetoacetate and beta-hydroxybutyrate) across cell membranes. These monocarboxylates play essential roles in carbohydrate, amino acid, and fat metabolism in mammalian cells, and must be rapidly transported across plasma membrane of cells. MCTs catalyse the transport of these solutes via a facilitative diffusion mechanism that requires co-transport of protons. Monocarboxylates such as lactate, pyruvate, and ketone bodies play a central role in cellular metabolism and metabolic communications among tissues. Lactate is the end product of aerobic glycolysis. Lactate has recently emerged as a critical regulator of cancer development, invasion, and metastasis. Tumor lactate levels correlate well with metastasis, tumor recurrence, and poor prognosis (J. Clin. Invest 2013).

MCTs are 12-span transmembrane proteins with N- and C-terminus in cytosolic domain, and are members of solute carrier SLC16A gene family. MCT family contains 14 members, and so far MCT1, MCT2, MCT3, and MCT4 are well characterized [Biochemical Journal (1999), 343:281-299].

Regulation and function of MCT1 and MCT4 are dependent upon interaction of other protein such as the chaperone CD147 (basigin, EMMPRIN), a member of immunoglobulin super family with a single transmembrane helix. Many studies have shown the tight association of CD147 and MCT1 and MCT4 [Future Oncology (2010), (1), 127]. CD147 acts as a chaperone to bring MCT1 and MCT4 to the plasma membrane and remain closely associated for the essential function of MCTs.

Malignant tumors contain aerobic and hypoxic regions, and the hypoxia increases the risk of cancer invasion and metastasis. Tumor hypoxia leads to treatment failure, relapse, and patient mortality as these hypoxic cells are generally resistant to standard chemo- and radiation therapy. In regions of hypoxia, cancer cells metabolize glucose into lactate whereas nearby aerobic cancer cells take up this lactate via the MCT1 for oxidative phosphorylation (OXPHOS). Under hypoxic conditions, cancer cells up regulate glucose transporters and consume large quantities of glucose. Cancer cells also up regulate glycolytic enzymes and convert glucose into lactate, which is then efflux out of cell via MCT4. The nearby aerobic cancer cells take up this lactate via MCT1 for energy generation through OXPHOS. Thus, the limited glucose availability to the tumor is used most efficiently via synergistic metabolic symbiosis. This utilization of lactate as an energy substitute for survival prevents the aerobic cells from consuming large quantities of glucose.

### SUMMARY OF THE INVENTION

In one aspect, the invention relates to compounds that are effective as monocarboxylate transport modulators. Such compounds have of formula I:

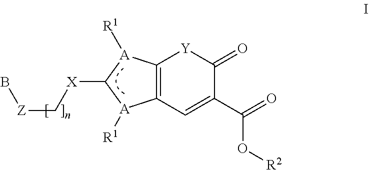

wherein:
n is 0, 1, or 2;
X is either O, or NR″;
Y is either O, or NR″;
Z is a bond, CH₂, C═O, SO₂;

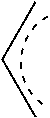

is either

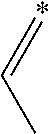

or

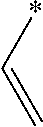 [SMILES: *C=C]

resonance isomers;
A is a nitrogen (N), sulfur (S), oxygen (O), or a carbon (C) atom optionally substituted by H or R″ substituent;
R¹ is independently selected from the group consisting of hydrogen, halogen (Br, F, I, Cl), alkyl, —CHF₂, —CF₃, —CN, —C(O)R″, —C(O)OR″, —SO₂R″, —C(O)NR″₂, and —C(O)N(OR″)R″,

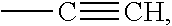

with the proviso that when A is O or S, R¹ does not exist;
R² is independently selected from the group of hydrogen, —C(O)R″, —(CH₂)₀₋₄C(O)R″, —(CH₂)₀₋₄C(O)OR″, or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur;
B is a ring selected from a 3-8 membered saturated or partially unsaturated monocyclic carbocyclic ring, phenyl, an 8-10 membered bicyclic aryl ring, a 3-8 membered saturated or partially unsaturated monocyclic or bicyclic heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur, wherein B is optionally substituted with one or more R″ substituents;
R″ is hydrogen or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur.

### DETAILED DESCRIPTION

In certain embodiments, the present invention relates to a compound of formula I:

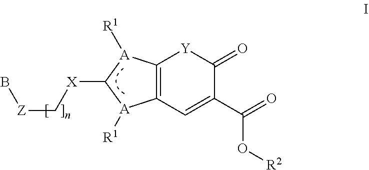

wherein:

n is 0, 1, or 2;
X is either O or NR″;
Y is either O or NR″;
Z is a bond, CH₂, C═O, or SO₂;

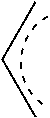

is either

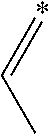

or

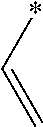 [SMILES: *C=C]

resonance isomers;
A is a nitrogen (N), sulfur (S), oxygen (O), or a carbon (C) atom optionally substituted by H or R″ substituent;
R¹ is independently selected from the group consisting of hydrogen, halogen (Br, F, I, Cl), alkyl, —CHF₂, —CF₃, —CN, —C(O)R″, —C(O)OR″, —SO₂R″, —C(O)NR″₂, and —C(O)N(OR″)R″,

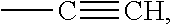

with the proviso that when A is O or S, R¹ does not exist;
R² is independently selected from the group of hydrogen, —C(O)R″, —(CH₂)₀₋₄C(O)R″, —(CH₂)₀₋₄C(O)OR″, or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur;
B is a ring selected from a 3-8 membered saturated or partially unsaturated monocyclic carbocyclic ring, phenyl, an 8-10 membered bicyclic aryl ring, a 3-8 membered saturated or partially unsaturated monocyclic or bicyclic heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur, wherein B is optionally substituted with one or more R″ substituents;
R″ is hydrogen or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur.

Compounds described herein and pharmaceutically acceptable compositions thereof, are useful for treating a variety of diseases, disorders or conditions, associated with abnormal cellular responses triggered by altered cellular metabolism. Such diseases, disorders, or conditions include those described below.

Compounds provided by this invention are also useful for the study of monocarboxylate transport modulation in biological and pathological phenomena; the study of intracellular and intercellular signal transduction pathways mediated by lactate and other monocarboxylates, and the comparative evaluation of new monocarboxylate transport modulators.

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention and claims that follow.

As used herein, the following definitions shall apply unless otherwise indicated. For purposes of this invention, the chemical elements are identified in accordance with the Periodic Table of the Elements CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

Compounds of the present invention may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers or enantiomers, with all possible isomers and mixtures thereof, including optical isomers, being included in the present invention.

Generally, reference to a certain element such as hydrogen or H is meant (if appropriate) to include all isotopes of that element, for example, deuterium and tritium for hydrogen.

The term “alkyl” as used herein means a straight- or branched-chain hydrocarbon having from one to eight carbon atoms, and includes, for example, and without being limited thereto, methyl, ethyl, propyl, isopropyl, t-butyl and the like. Substituted alkyl includes, for example, and without being limited thereto, haloalkyl, hydroxyalkyl, cyanoalkyl, and the like. This is applied to any of the groups mentioned herein, such as substituted “alkenyl”, “alkynyl”, “aryl”, etc.

The term “alkenyl” as used herein means a straight- or branched-chain aliphatic hydrocarbon having at least one double bond. The alkene may have from two to eight carbon atoms, and includes, for example, and without being limited thereto, ethenyl, 1-propenyl, 1-butenyl and the like. The term “alkenyl” encompass radicals having “cis” and “trans” orientations, or alternatively, “E” and “Z” orientations.

The term “alkynyl” as used herein means a straight- or branched-chain aliphatic hydrocarbon having at least one triple bond. The alkyne may have from two to eight carbon atoms, and includes, for example, and without being limited thereto, 1-propynyl (propargyl), 1-butynyl and the like.

The term “cycloalkyl” as used herein means an aliphatic carbocyclic system (which may be unsaturated) containing one or more rings wherein such rings may be attached together in a pendent manner or may be fused. In one aspect, the ring(s) may have from three to seven carbon atoms, and includes, for example, and without being limited thereto, cyclopropyl, cyclohexyl, cyclohexenyl and the like.

The term “heterocycloalkyl” as used herein means a heterocyclic system (which may be unsaturated) having at least one heteroatom selected from N, S and/or O and containing one or more rings wherein such rings may be attached together in a pendent manner or may be fused. In one aspect, the ring(s) may have a three- to seven-membered cyclic group and includes, for example, and without being limited thereto, piperidinyl, piperazinyl, pyrrolidinyl, tetrahydrofuranyl, tetrahydropyranyl and the like.

The term “heteroatom” means one or more of oxygen, sulfur, nitrogen, phosphorus, or silicon.

The term “unsaturated”, as used herein, means that a moiety has one or more units of unsaturation.

The term “alkoxy” as used herein means a straight- or branched-chain oxygen-containing hydrocarbon; in one aspect, having from one to eight carbon atoms and includes, for example, and without being limited thereto, methoxy, ethoxy, propyloxy, isopropyloxy, t-butoxy and the like.

The term “halo” or “halogen” includes, for example, and without being limited thereto, fluoro, chloro, bromo, and iodo, in both radioactive and non-radioactive forms.

The term “alkylene” as used herein means a difunctional branched or unbranched saturated hydrocarbon; in one aspect, having one to eight carbon atoms, and includes, for example, and without being limited thereto, methylene, ethylene, n-propylene, n-butylene and the like.

The term “aryl”, alone or in combination, as used herein means a carbocyclic aromatic system containing one or more rings. In particular embodiments, aryl is one, two or three rings.

In one aspect, the aryl has five to twelve ring atoms. The term “aryl” encompasses aromatic radicals such as phenyl, naphthyl, tetrahydronaphthyl, indanyl, biphenyl, phenanthryl, anthryl or acenaphthyl. The “aryl” group may have 1 to 4 substituents such as lower alkyl, hydroxyl, halo, haloalkyl, nitro, cyano, alkoxy, lower alkylamino and the like.

The term “heteroaryl”, alone or in combination, as used herein means an aromatic system having at least one heteroatom selected from N, S and/or O and containing one or more rings. In particular embodiments, heteroaryl is one, two or three rings. In one aspect, the heteroaryl has five to twelve ring atoms. The term “heteroaryl” encompasses heteroaromatic groups such as triazolyl, imidazolyl, pyrrolyl, tetrazolyl, pyridyl, indolyl, furyl, benzofuryl, thienyl, benzothienyl, quinolyl, oxazolyl, thiazolyl and the like. The “heteroaryl” group may have 1 to 4 substituents such as lower alkyl, hydroxyl, halo, haloalkyl, nitro, cyano, alkoxy, lower alkylamino and the like.

It is understood that substituents and substitution patterns on the compounds of the invention may be selected by one of ordinary skill in the art to provide compounds that are chemically stable and that can be readily synthesized by techniques known in the art, as well as those methods set forth below. If a substituent is itself substituted with more than one group, it is understood that these multiple groups may be on the same carbon or on different carbons, as long as a stable structure results.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘)}₂; SiR^{∘}₃; —(C₁₋₄ straight or branched)alkylene)O—N(R^{∘)}₂; or —(C₁₋₄ straight or branched)alkylene)C(O)O—N(R^{∘)}₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R_{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this invention include those derived from suitable inorganic and organic acids and bases.

As used herein, and as would be understood by the person of skill in the art, the recitation of “a compound”—unless expressly further limited—is intended to include salts of that compound. Thus, for example, the recitation “a compound of formula I” as depicted above, in which R² is H, would include salts in which the carboxylic acid is of the formula COO⁻M⁺, wherein M is any counterion. In a particular embodiment, the term “compound of formula I” refers to the compound or a pharmaceutically acceptable salt thereof. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

The term “stereoisomers” is a general term for all isomers of the individual molecules that differ only in the orientation of their atoms in space. It includes mirror image isomers (enantiomers), geometric (cis/trans) isomers and isomers of compounds with more than one chiral centre that are not mirror images of one another (diastereomers).

The term “treat” or “treating” means to alleviate symptoms, eliminate the causation of the symptoms either on a temporary or permanent basis, or to inhibit or slow the appearance of symptoms of the named disorder or condition. The term “therapeutically effective amount” means an amount of the compound which is effective in treating or lessening the severity of one or more symptoms of a disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to the patient. One example of such a carrier is pharmaceutically acceptable oil typically used for parenteral administration.

When introducing elements disclosed herein, the articles “a”, “an”, “the”, and “said” are intended to mean that there are one or more of the elements. The terms “comprising”, “having”, “including” are intended to be open-ended and mean that there may be additional elements other than the listed elements.

According to one aspect, the present invention relates to a compound of formula I,

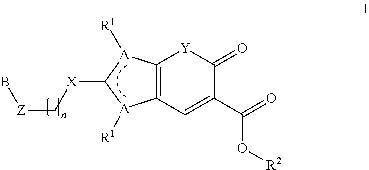

wherein:

n is 0, 1, or 2;
X is either O, NR″;
Y is either O, or NR″;
Z is a bond, CH₂, C═O, SO₂;

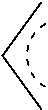

is either

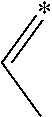 [SMILES: *=CC]

or

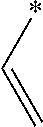 [SMILES: *C=C]

resonance isomers;
A is a nitrogen (N), sulfur (S), oxygen (O), or a carbon (C) atom optionally substituted by H or R″ substituent;
R¹ is independently selected from the group consisting of hydrogen, halogen (Br, F, I, Cl), alkyl, —CHF₂, —CF₃, —CN, —C(O)R″, —C(O)OR″, —SO₂R″, —C(O)NR″₂, and —C(O)N(OR″)R″,

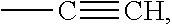

with the proviso that when A is O or S, R¹ does not exist;
R² is independently selected from the group of hydrogen, —C(O)R″, —(CH₂)₀₋₄C(O)R″, —(CH₂)₀₋₄C(O)OR″, or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur;
B is a ring selected from a 3-8 membered saturated or partially unsaturated monocyclic carbocyclic ring, phenyl, an 8-10 membered bicyclic aryl ring, a 3-8 membered saturated or partially unsaturated monocyclic or bicyclic heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur, wherein B is optionally substituted with one or more R″ substituents;
R″ is hydrogen or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur.

In some embodiments, the compound has the following structure:

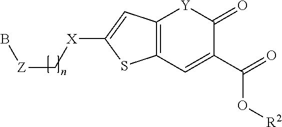

wherein:
n is 0, 1, or 2;
Z is a bond, CH₂, C═O, SO₂;
X is either O, or NR″;
Y is either O, or NR″;
R² is independently selected from the group of hydrogen, —C(O)R″, —(CH₂)₀₋₄C(O)R″, —(CH₂)₀₋₄C(O)OR″, or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur;
B is a ring selected from a 3-8 membered saturated or partially unsaturated monocyclic carbocyclic ring, phenyl, an 8-10 membered bicyclic aryl ring, a 3-8 membered saturated or partially unsaturated monocyclic or bicyclic heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur, wherein B is optionally substituted with one or more R″ substituents;
R″ is hydrogen or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur;

In one embodiment, n is equal to 1.

In some embodiments, Z is a “bond”.

In other embodiments, compound has the following structure;

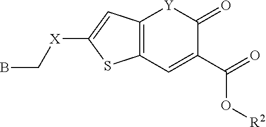

wherein:

X is either O or NR″;
Y is either O or NR″; R² is independently selected from the group of hydrogen, —C(O)R″, —(CH₂)₀₋₄C(O)R″, —(CH₂)₀₋₄C(O)OR″, or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur; B is a ring selected from a 3-8 membered saturated or partially unsaturated monocyclic carbocyclic ring, phenyl, an 8-10 membered bicyclic aryl ring, a 3-8 membered saturated or partially unsaturated monocyclic or bicyclic heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur, wherein B is optionally substituted with one or more R″ substituents;
R″ is hydrogen or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur;

In one embodiment, Y is oxygen.

In other embodiments, R² is hydrogen.

In a further embodiment, the base addition salt is formed from sodium, potassium, magnesium, calcium.

In some embodiments, the compound has the following structure:

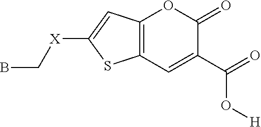

wherein each of B, and X, is as defined above and described herein.

In one embodiment, X is nitrogen.

In a further embodiment, the base addition salt is formed from sodium, potassium, magnesium, calcium.

In some embodiments, the compound has the following structure:

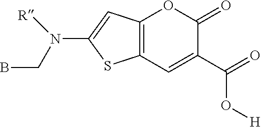

wherein each of B and R″ are as defined above and described herein.

In some embodiments, R is an alkyl (e.g., methyl). In some embodiments, B is substituted or unsubstituted phenyl. In some embodiments, B is substituted or unsubstituted heteroaryl (e.g., pyridyl) or 3-8 membered saturated monocyclic carbocyclic or heterocyclic ring.

In some embodiments, the compound is selected from;

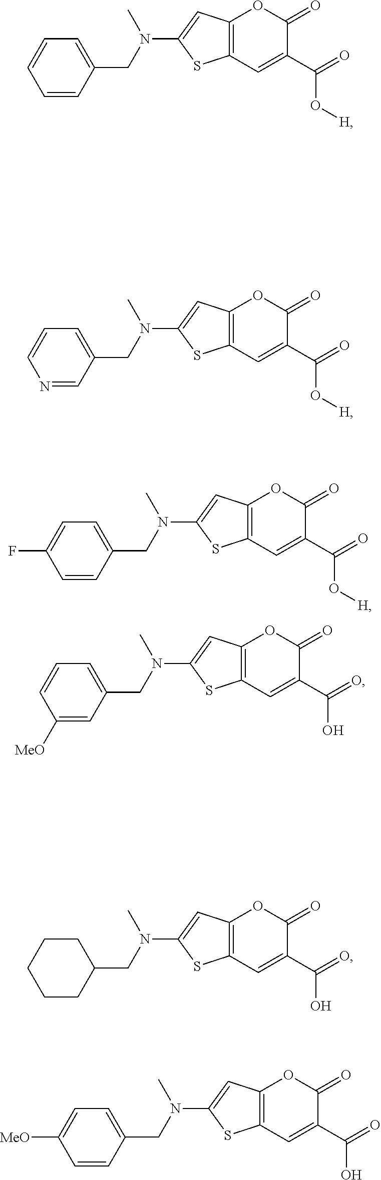

In some embodiments, the compound has the following structure:

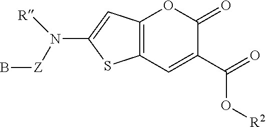

wherein:

### Z is —O or SO₂;

R² is selected from the group of hydrogen, —C(O)R″, —(CH₂)₀₋₄C(O)R″, —(CH₂)₀₋₄C(O)OR″, or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur;
B is a ring selected from a 3-8 membered saturated or partially unsaturated monocyclic carbocyclic ring, phenyl, an 8-10 membered bicyclic aryl ring, a 3-8 membered saturated or partially unsaturated monocyclic or bicyclic heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur, wherein B is optionally substituted with one or more R″ substituents;
R″ is hydrogen or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur.

In other embodiments, compound has the following structures:

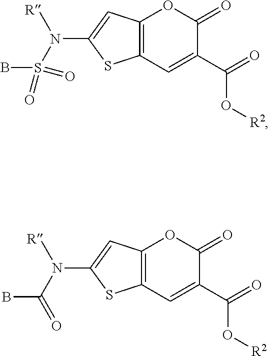

wherein:
R² is selected from the group of hydrogen, —C(O)R″, —(CH₂)₀₋₄C(O)R″, —(CH₂)₀₋₄C(O)OR″, or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur;
B is a ring selected from a 3-8 membered saturated or partially unsaturated monocyclic carbocyclic ring, phenyl, an 8-10 membered bicyclic aryl ring, a 3-8 membered saturated or partially unsaturated monocyclic or bicyclic heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur, wherein B is optionally substituted with one or more R″ substituents;
R″ is hydrogen or an optionally substituted group selected from C₁₋₆ alkyl, 3-8 membered saturated or partially unsaturated cycloalkyl ring, 3-8 membered saturated or partially unsaturated heterocycloalkyl ring having 1-2 heteroatoms independently selected from nitrogen, oxygen, and sulfur, phenyl, or a 5-6 membered heteroaryl ring having 1-4 heteroatoms independently selected from nitrogen, oxygen, and sulfur;

In other embodiments, compound has the following structures:

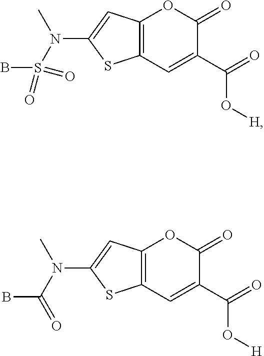

wherein:
B is a ring selected from a 3-8 membered saturated or partially unsaturated monocyclic carbocyclic ring, phenyl, an 8-10 membered bicyclic aryl ring, a 3-8 membered saturated or partially unsaturated monocyclic or bicyclic heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or an 8-10 membered bicyclic heteroaryl ring having 1-4 heteroatoms independently selected from nitrogen, oxygen, and sulfur, wherein B is optionally substituted with one or more R″ substituents;

In some embodiments, the compound is selected from;

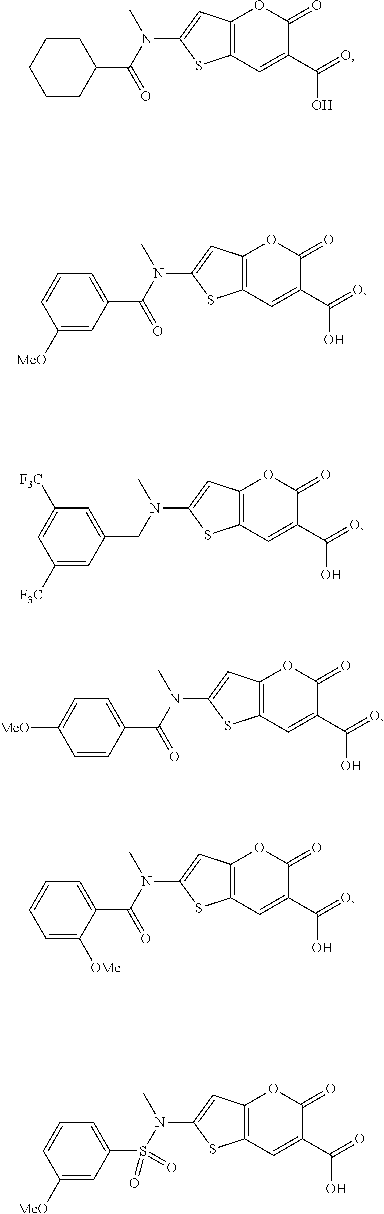 [SMILES: CN(C(=O)C1CCCCC1)c1cc2oc(=O)c(C(=O)O)cc2s1 ; COc1cccc(C(=O)N(C)c2cc3oc(=O)c(C(=O)O)cc3s2)c1 ; CN(Cc1cc(C(F)(F)F)cc(C(F)(F)F)c1)c1cc2oc(=O)c(C(=O)O)cc2s1 ; COc1ccc(C(=O)N(C)c2cc3oc(=O)c(C(=O)O)cc3s2)cc1 ; COc1ccccc1C(=O)N(C)c1cc2oc(=O)c(C(=O)O)cc2s1 ; COc1cccc(S(=O)(=O)N(C)c2cc3oc(=O)c(C(=O)O)cc3s2)c1]

In some embodiments, the compound has the following structure:

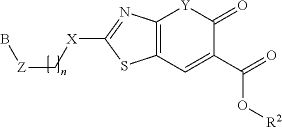

wherein, each of X, Y, Z, n, R², and B are as defined as above.

In one embodiment, n is equal to 1.

In other embodiments, Z is a “bond”.

In some embodiments, the compound has the following structure:

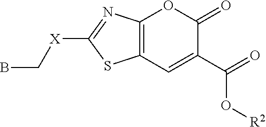

wherein, each of X and B are as defined as above.

In one embodiment, R² is hydrogen.

In some embodiments, the compound has the following structure:

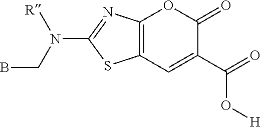

wherein each of B and R″ are as defined above and described herein.

In some embodiments, R is an alkyl (e.g., methyl). In some embodiments, B is substituted or unsubstituted phenyl. In some embodiments, B is substituted or unsubstituted heteroaryl (e.g., pyridyl) or 3-8 membered saturated monocyclic carbocyclic or heterocyclic ring.

In some embodiments, the compound is selected from;

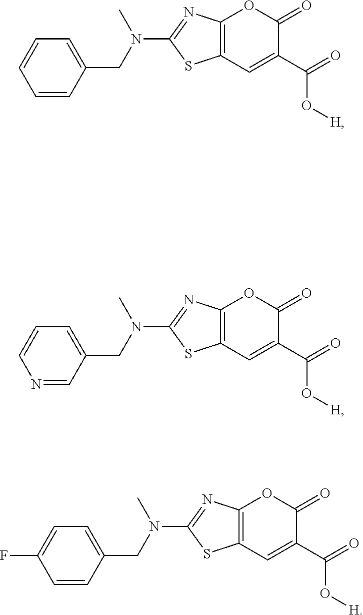

In one aspect, the invention features a pharmaceutical composition comprising a compound described herein, and a pharmaceutically acceptable carrier.

In another aspect, the invention features a method of treating a neoplastic or metabolic disorder in a subject, comprising administering a pharmaceutically effective amount of a compound, prodrug thereof, or composition described herein.

Also provided herein are methods of treating a disease associated with expression or activity of MCT1, MCT2, MCT3, MCT4, CD147, NFkB, p53 in a subject comprising administering to the patient a therapeutically effective amount of a compound described herein. For example, provided herein are methods of treating various cancers in mammals specifically including humans, dogs, cats, and farm animals, including hematologic malignancies (leukemias, lymphomas, myelomas, myelodysplastic and myeloproliferative syndromes) and solid tumors (carcinomas such as prostate, breast, lung, colon, pancreatic, renal, brain, CNS, skin, cervical, ovarian as well as soft tissue and osteo-sarcomas, and stromal tumors), inflammatory disorders such as rheumatoid arthritis, osteoarthritis, psoriatic arthritis, multiple sclerosis, systemic lupus, systemic sclerosis, vasculitis syndromes (small, medium and large vessel), atherosclerosis, psoriasis and other dermatological inflammatory disorders (such as pemphigous, pemphigoid, allergic dermatitis), and urticarial syndromes comprising administering a compound represented by formula I.

Also provided are compounds represented by formula I for use in therapy and/or for the manufacture of a medicament for the treatment of a disease associated with expression or activity of MCT1, MCT2, MCT3, MCT4, CD147, NFkB, p53 in a subject.

In yet another aspect, the compound or composition is administrable intravenously and/or intraperitoneally and/or orally.

In some embodiments, the present invention provides a compound selected from:
2-(benzyl(methyl)amino)-5-oxo-5H-thieno[3,2-b]pyran-6-carboxylic acid
2-((4-fluorobenzyl)(methyl)amino)-5-oxo-5H-thieno[3,2-b]pyran-6-carboxylic acid
2-((3-fluorobenzyl)(methyl)amino)-5-oxo-5H-thieno[3,2-b]pyran-6-carboxylic acid
2-((3-methoxybenzyl)(methyl)amino)-5-oxo-5H-thieno[3,2-b]pyran-6-carboxylic acid
2-((cyclohexylmethyl)(methyl)amino)-5-oxo-5H-thieno[3,2-b]pyran-6-carboxylic acid
2-(methyl(3-(trifluoromethyl)benzyl)amino)-5-oxo-5H-thieno[3,2-b]pyran-6-carboxylic acid
2-((3,5-bis(trifluoromethyl)benzyl)(methyl)amino)-5-oxo-5H-thieno[3,2-b]pyran-6-carboxylic acid
2-(methyl((tetrahydro-2H-pyran-4-yl)methyl)amino)-5-oxo-5H-thieno[3,2-b]pyran-6-carboxylic acid
2-(3-methoxy-N-methylbenzamido)-5-oxo-5H-thieno[3,2-b]pyran-6-carboxylic acid
2-(N-methylcyclohexanecarboxamido)-5-oxo-5H-thieno[3,2-b]pyran-6-carboxylic acid
2-(4-fluoro-N-methylbenzamido)-5-oxo-5H-thieno[3,2-b]pyran-6-carboxylic acid
2-(methyl((1-methylpiperidin-4-yl)methyl)amino)-5-oxo-5H-thieno[3,2-b]pyran-6-carboxylic acid
2-(methyl(pyridin-3-ylmethyl)amino)-5-oxo-5H-thieno[3,2-b]pyran-6-carboxylic acid
2-(methyl(thiophen-2-ylmethyl)amino)-5-oxo-5H-thieno[3,2-b]pyran-6-carboxylic acid
2-[Methyl-(3-trifluoromethyl-benzoyl)-amino]-5-oxo-5H-thieno[3,2-b]pyran-6-carboxylic acid
2-[(4-Methoxy-benzoyl)-methyl-amino]-5-oxo-5H-thieno[3,2-b]pyran-6-carboxylic acid
2-[(2-Methoxy-benzoyl)-methyl-amino]-5-oxo-5H-thieno[3,2-b]pyran-6-carboxylic acid
2-[Methyl-(tetrahydro-pyran-4-carbonyl)-amino]-5-oxo-5H-thieno[3,2-b]pyran-6-carboxylic acid
2-[(4-Fluoro-3-methoxy-benzoyl)-methyl-amino]-5-oxo-5H-thieno[3,2-b]pyran-6-carboxylic acid
2-[Methyl-(4-trifluoromethyl-benzoyl)-amino]-5-oxo-5H-thieno[3,2-b]pyran-6-carboxylic acid
2-[(4-Methoxy-benzyl)-methyl-amino]-5-oxo-5H-thieno[3,2-b]pyran-6-carboxylic acid
2-[(2-Methoxy-benzyl)-methyl-amino]-5-oxo-5H-thieno[3,2-b]pyran-6-carboxylic acid
2-[Methyl-(4-trifluoromethyl-benzyl)-amino]-5-oxo-5H-thieno[3,2-b]pyran-6-carboxylic acid
2-[(3-Methoxy-benzenesulfonyl)-methyl-amino]-5-oxo-5H-thieno[3,2-b]pyran-6-carboxylic acid
2-[(4-Methoxy-benzenesulfonyl)-methyl-amino]-5-oxo-5H-thieno[3,2-b]pyran-6-carboxylic acid
2-[(4-Fluoro-benzenesulfonyl)-methyl-amino]-5-oxo-5H-thieno[3,2-b]pyran-6-carboxylic acid
2-[Methyl-(4-trifluoromethyl-benzenesulfonyl)-amino]-5-oxo-5H-thieno[3,2-b]pyran-6-carboxylic acid
2-[Methyl-(3-trifluoromethyl-benzenesulfonyl)-amino]-5-oxo-5H-thieno[3,2-b]pyran-6-carboxylic acid.

In one aspect, the invention relates to a composition comprising a compound of this invention or a pharmaceutically acceptable derivative thereof and a pharmaceutically acceptable carrier, adjuvant, or vehicle. The amount of compound in compositions of this invention is such that is effective to measurably inhibit monocarboxylate transport, in a biological sample or in a patient. In certain embodiments, the amount of compound in compositions of this invention is such that is effective to measurably inhibit monocarboxylate transport in a biological sample or in a patient. In certain embodiments, a composition of this invention is formulated for administration to a patient in need of such composition. In some embodiments, a composition of this invention is formulated for oral administration, intravenous, subcutaneous, intraperitoneal or dermatological application to a patient.

The term “patient”, as used herein, means an animal. In some embodiments, the animal is a mammal. In certain embodiments, the patient is a veterinary patient (i.e., a non-human mammal patient). In some embodiments, the patient is a dog. In other embodiments, the patient is a human.

Compounds and compositions described herein are generally useful for the inhibition of monocarboxylate transport. The activity of a compound utilized in this invention as an inhibitor of monocarboxylate transport may be assayed in vitro, in vivo or in a cell line. Detailed conditions for assaying a compound utilized in this invention as an inhibitor of monocarboxylate transport are set forth in the Examples below.

The compounds and compositions described herein can be administered to cells in culture, e.g. in vitro or ex vivo, or to a subject, e.g., in vivo, to treat, prevent, and/or diagnose a variety of disorders, including those described herein below.

As used herein, the term “treat” or “treatment” is defined as the application or administration of a compound, alone or in combination with, a second compound to a subject, e.g., a patient, or application or administration of the compound to an isolated tissue or cell, e.g., cell line, from a subject, e.g., a patient, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder).

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, the term “subject” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, cow, pig, etc, and companion animals (dog, cat, horse etc).

Provided compounds are inhibitors of monocarboxylate transport and are therefore useful for treating one or more disorders associated with activity of monocarboxylate transport. Thus, in certain embodiments, the present invention provides a method for treating a monocarboxylate transport-mediated disorder comprising the step of administering to a patient in need thereof a compound of the present invention, or pharmaceutically acceptable composition thereof.

As used herein, the term “monocarboxylate transport-mediated” disorder or condition, as used herein, means any disease or other deleterious condition in which monocarboxylate transport is known to play a role. Accordingly, another embodiment of the present invention relates to treating or lessening the severity of one or more diseases in which monocarboxylate transport is known to play a role. Specifically, the present invention relates to a method of treating or lessening the severity of a disease or condition selected from a proliferative disorder, wherein said method comprises administering to a patient in need thereof a compound or composition according to the present invention. Such disorders are set forth in detail below.

### Neoplastic Disorders

A compound or composition described herein can be used to treat a neoplastic disorder. A “neoplastic disorder” is a disease or disorder characterized by cells that have the capacity for autonomous growth or replication, e.g., an abnormal state or condition characterized by proliferative cell growth. Exemplary neoplastic disorders include: carcinoma, sarcoma, metastatic disorders (e.g., tumors arising from prostate, colon, lung, breast, cervical, ovarian, liver, melanoma, brain, CNS, head and neck, osteosarcoma, gastrointestinal, pancreatic, hematopoietic neoplastic disorders, e.g., leukemias, lymphomas, myeloma and other malignant plasma cell disorders, and metastatic tumors. Prevalent cancers include: breast, prostate, colon, lung, liver, and pancreatic cancers. Treatment with the compound may be in an amount effective to ameliorate at least one symptom of the neoplastic disorder, e.g., reduced cell proliferation, reduced tumor mass, etc.

The disclosed methods are useful in the prevention and treatment of cancer, including for example, solid tumors, soft tissue tumors, and metastases thereof, as well as in familial cancer syndromes such as Li Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. Exemplary cancers described by the National Cancer Institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated or prevented in accordance with the methods described herein.

In some embodiments, a compound described herein is administered together with an additional cancer treatment. Exemplary cancer treatments include, for example: chemotherapy, targeted therapies such as antibody therapies, kinase inhibitors, immunotherapy, immune checkpoint inhibitors, cancer metabolism therapies, hormonal therapy, and anti-angiogenic therapies.

Immune Activation in the Tumor Microenvironment

In some embodiments, a compound described herein may be used to activate immune cells in the tumor leading to cancer cell killing. Lactate is a metabolite produced from cancer cell metabolism, which suppress the immune system in the local tumor microenvironment. A compound described herein may decrease the lactate content in the tumor microenvironment thus preventing and immune suppression.

Compounds and methods described herein may be used to prevent or treat a disease or disorder associated with angiogenesis. Diseases associated with angiogenesis include cancer, cardiovascular diseases and muscular degeneration. Angiogenesis is the physiological processes involving the growth of new vessels from pre-existing blood vessels. Angiogenesis is the normal and vital process in growth and development, as well as in wound healing and in granular tissue. However, it is also a fundamental step in the transition of tumors from a dormant state to a malignant one. Angiogenesis may be a target for combating diseases characterized by either poor vascularization or abnormal vasculature.

Application of specific compounds that may inhibit the creation of new blood vessels in the body may help combat such diseases. The presence of blood vessels, where there should be none, may affect the normal properties of a tissue, increasing the likelihood of failure. The absence of blood vessels in a repairing or otherwise metabolically active tissue may inhibit repair or other essential functions. Several diseases such as ischemic chronic wounds are the results of failure or insufficient blood vessel formation and may be treated by a local expansion of blood vessels, thus bringing new nutrients to the site, facilitating repair. Other diseases such as age-related muscular degeneration may be created by a local expansion of blood vessels, interfering with normal physiological processes.

Vascular endothelial growth factor (VEGF) has been demonstrated to be a major contributor to angiogenesis, increasing the number of capillaries in a given network. Upregulation of VEGF is a major component of the physiological response to exercise and its role in angiogenesis is suspected to be a possible treatment for vascular injuries. In vitro studies clearly demonstrated that VEGF is a potent stimulator of angiogenesis because, in the presence of this growth factor, plated endothelial cells will proliferate and migrate, eventually forming tube structures resembling capillaries.

Tumors induce blood vessel growth by secreting various growth factors (e.g. VEGF). Growth factors such as bFGF and VEGF can induce capillary growth into the tumor, which some researchers suspect supply required nutrients allowing for tumor expansion.

Angiogenesis represents an excellent target for the treatment of cancer and cardiovascular diseases. It is a potent physiological process that underlies the natural manner in which our bodies responds to a diminution of blood supply to vital organs, namely the production of new collateral vessels to overcome the ischemic insult.

Overexpression of VEGF causes increased permeability in blood vessels in addition to stimulating angiogenesis. In wet muscular degeneration, VEGF causes proliferation of capillaries into the retina. Since the increase in angiogenesis also causes edema, blood and other retinal fluids leak into the retina causing loss of vision.

Antiangiogenic therapy can include kinase inhibitors targeting vascular endothelial growth factor (VEGF) such as sutinib, sorafenib, monoclonal antibodies, receptor “decoys” to VEGF, VEGF-Trap, thalidomide, its analogs (lenalidomide, pomalidomide), agents targeting non-VEGF angiogenic targets such as fibroblast growth factor (FGF), angiopoietins, angiostatin, or endostatin.

The body's immune system detects foreign objects and organisms such as bacteria, virus, and other pathogens, and protects the body by eliminating those harmful matters. Sometimes, those immune system responses against foreign pathogens or tissues become more harmful to the host, for example, allergies to food and extrinsic antigens such as pollen and respiratory diseases such as asthma. In addition, strong responses against transplant tissues or organs occur leading to the rejection of them. In such cases, immunosuppressive drugs are needed to avoid those complications.

Additionally, the body's immune system does not exert responses against self-tissues or self-antigens under normal circumstances. However, in some cases, body exerts a strong immune response against self-tissues aggressively leading to a variety of autoimmune diseases such as rheumatoid arthritis, multiple sclerosis, type I diabetes, etc. Most immune responses are initiated and controlled by T helper lymphocytes, which respond to antigens.

A number of immunosuppressive therapies have been developed over the last decades. These include rapamycin, which disrupts the cytokine such as IL-2-driven T-cell proliferation by interfering with TOR (Target of Rapamycin) function. However, rapamycin has been shown to cause significant side effects including hyperlipidemia (Hong et al, Semin. Nephrol., 10(2); 108-125, 2000).

Compounds and compositions described herein may also be used to treat selectively sub-population of patients who express either MCT1 or MCT4 or both. It is known that a patient's response to a drug may be dependent upon patient's genetic profile and/or the type of the disease. It has been demonstrated that MCT4 is a biomarker that predicts poor overall survival of aggressive triple negative breast cancer patients.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### ABBREVIATIONS

atm Atmosphere
aq. Aqueous
BINAP 2,2′-bis(diphenylphosphino)-1,1′-binaphthyl
Boc tert-butoxycarbonyl
CH₃CN Acetonitrile
CDI N,N′-Carbonyldiimidazole
DCC N,N-Dicyclohexylcarbodiimide
DCM dichloromethane
DBU Diaza(1,3)bicyclo[5.4.0]undecane
DEA Diethylamine
DIEA N,N-Diisopropyl ethylamine
DIBAL-H Diisobutylaluminium hydride
DIC N,N′-Diisopropylcarbodiimide
DMAP N,N-Dimethyl-4-aminopyridine
DMF Dimethylformamide
DMSO Dimethylsulfoxide
DPPF Diphenylphosphinoferrocene
EA Ethyl acetate
EDCI N-[3-(dimethylamino)propyl]-N′-ethylcarbodiimide hydrochloride
EDC 1-Ethyl-3-(3-dimethylaminopropyl)carbodiimide
Et₂O Diethylether
EtOAc Ethyl acetate
EtOH Ethanol
EtI Iodoethane
Et Ethyl
FCC Flash Column chromatography
Fmoc 9-fluorenylmethyloxycarbonyl
h hour(s)
HetAr Heteroaryl
HOBt N-Hydroxybenzotriazole
HATU 1-[Bis(dimethylamino)methylene]-1H-1,2,3-triazolo[4,5-b]pyridinium 3-oxid hexafluorophosphate
HBTU O-(Benzotriazol-1-yl)-N,N,N′,N′-tetramethyluronium hexafluorophosphate
HPLC High performance liquid chromatography
L Leaving group
LAH Lithium aluminium hydride
LCMS HPLC mass spec
MCPBA m-Chlorobenzoic acid
MeCN Acetonitrile
MeOH Methanol
min Minutes
MeI Iodomethane
MeMgCl Methyl magnesium chloride
Me Methyl
n-BuLi 1-Butyllithium
NaOAc Sodium acetate
NMR Monocarboxylate magnetic resonance
NMP N-Methyl pyrrolidinone
nBuLi 1-Butyl lithium
o.n. Over night
RT, rt, r.t. Room temperature
TEA Triethylamine
THF Tetrahydrofuran
nBu normal Butyl
OMs Mesylate or methane sulfonate ester
OTs Tosylate, toluene sulfonate or 4-methylbenzene sulfonate ester
PCC Pyridinium chlorochromate
PPTS Pyridinium p-toluenesulfonate
TBAF Tetrabutylammonium fluoride
TLC Thin Layer Chromatography
TMSI Trimethylsilyliodide
pTsOHp-Toluenesulfonic acid
SPE Solid phase extraction (usually containing silica gel for mini-chromatography)
sat. Saturated
PG Protecting group
mins minutes

Throughout the following description of such processes it is to be understood that, where appropriate, suitable protecting groups will be added to, and subsequently removed from, the various reactants and Intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any Intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of Intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C. The term “reflux” shall mean, unless otherwise stated, in reference to an employed solvent a temperature at or above the boiling point of named solvent.

### General Synthetic Methods

Several general methods for preparing compounds of Formula I are illustrated in the following Schemes and Examples. Starting materials and the requisite Intermediates are in some cases commercially available or can be prepared according to literature procedures (Bioorg. Med. Chem. 16, 2008, 9487-9497; Med. Chem. Res. 2012; Asian J. Chem. 16, 2004, 1374-1380), or as illustrated herein. In the steps where product was obtained as a mixture of isomers, pure isomers can be easily separated using chromatographic methods in the literature.

It is understood that the functional groups present in compounds described in the Schemes below can be further manipulated, when appropriate, using the standard functional group transformation techniques available to those skilled in the art, to provide desired compounds described in this invention. Other variations or modifications, which will be obvious to those skilled in the art, are within the scope and teachings of this invention.

Certain bicyclic enone carboxylic acid compounds of Formula I, wherein the group B is selected from aryl and heteroaryl optionally substituted with one or more substituents and, the X is a nitrogen, n is 1, and the R″ group is an alkyl group can be prepared in accordance with an exemplary Scheme 1.

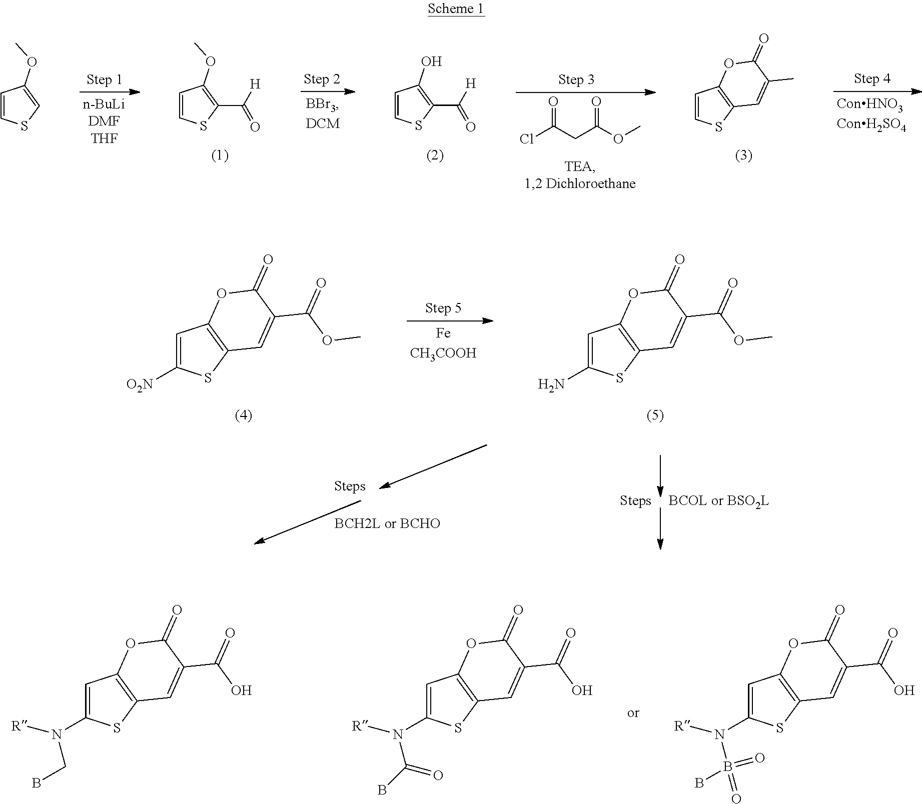

In Scheme 2, an exemplary general method is described for the preparation of certain bicyclic enone carboxylic acid compounds of Formula I, wherein the A is sulfur and nitrogen providing thiazole moiety.

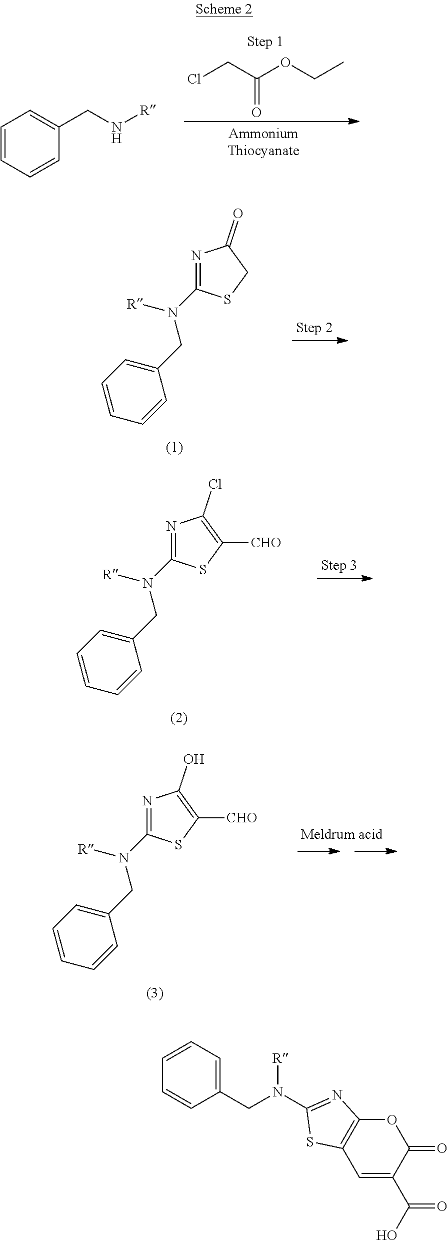

In Scheme 3, an exemplary general method is described for the preparation of certain bicyclic enone carboxylic acid compounds of Formula I, wherein the X is oxygen providing ether moiety.

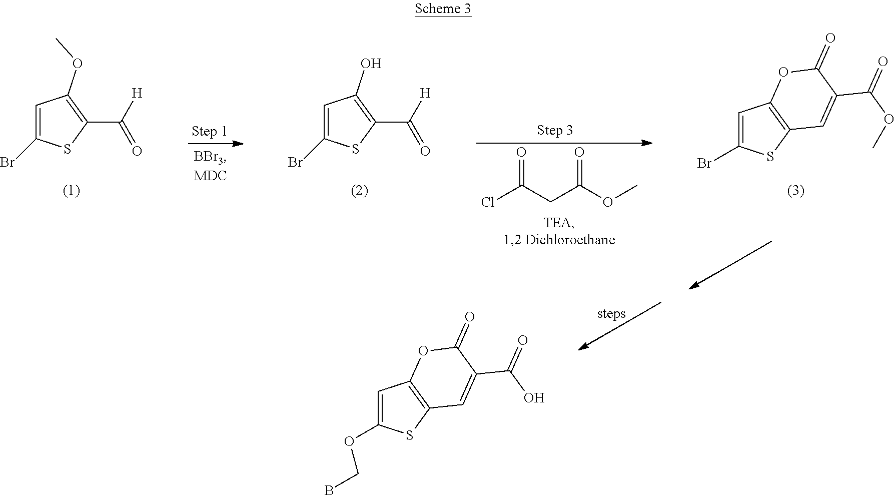

In Scheme 4, an exemplary general method is described for the preparation of certain bicyclic enone carboxylic acid compounds of Formula I, wherein the Y is nitrogen providing amide moiety.

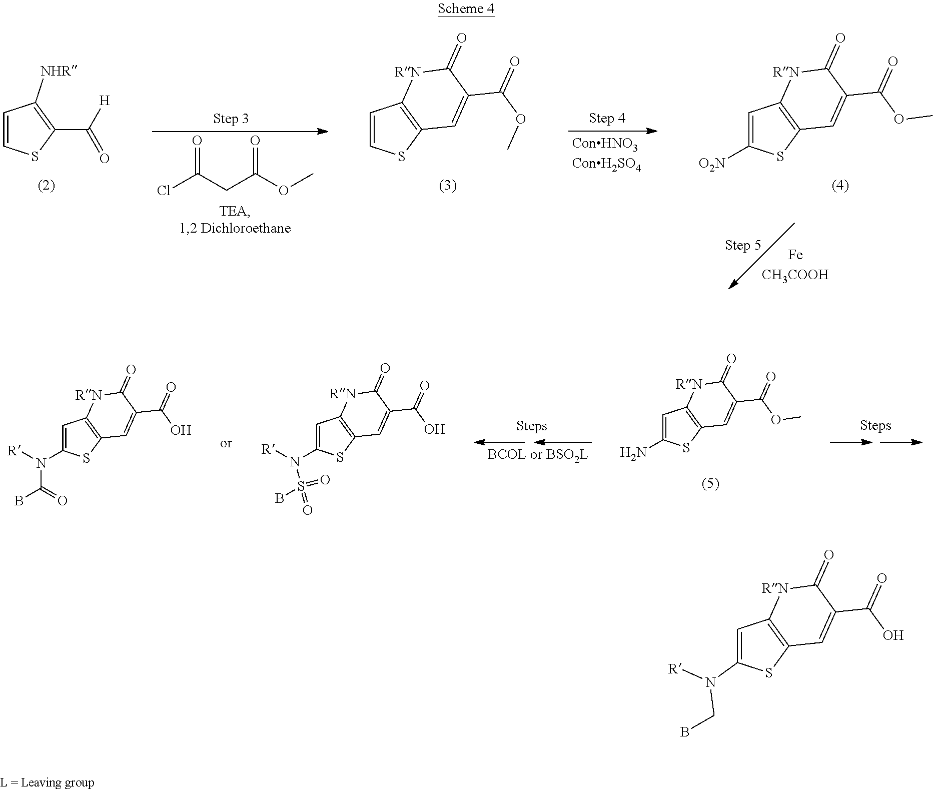

Preparation of Common Intermediate (5) in Scheme I:

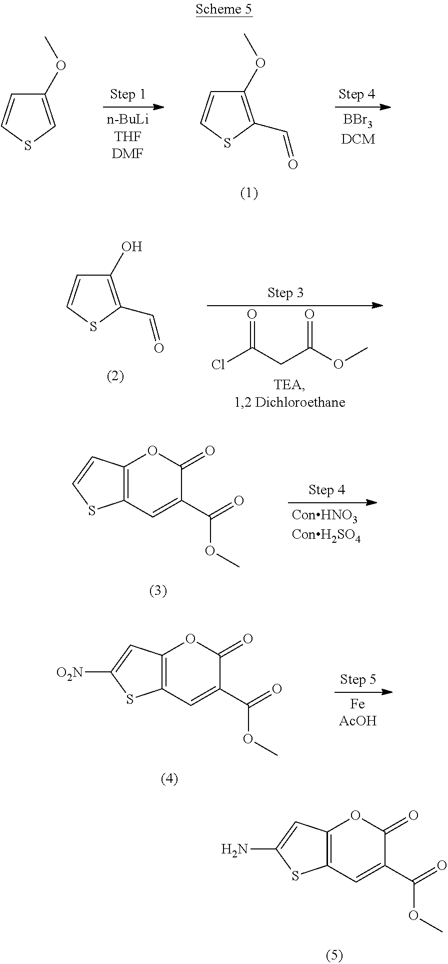

Step 1

In a 2 L, 3-necked round-bottom flask, 3-methoxythiophene (126 g, 1.11 mol) was charged into anhydrous THF (1.5 L) under nitrogen atmosphere. To this reaction mixture, n-BuLi (486 mL, 1.22 mol) was added dropwise at rt, and the resultant mixture was refluxed for 2 h, cooled to −10° C. followed by dropwise addition of DMF (105 g, 1.44 mol). Then reaction mixture was allowed to stir at rt overnight until the reaction completion monitored by TLC. To this reaction mixture was added saturated ammonium chloride solution, and the organic phase was collected. The aqueous phase was extracted with ethyl acetate, and the combined organic layer was dried over anhydrous sodium sulfate, concentrated under reduced pressure. The crude product was purified by silica gel column chromatography (5-15% EtOAc in Hexanes) to obtain 110 g of 3-methoxythiophene-2-carbaldehyde (1); Yield, 70.1%; ¹H NMR (400 MHz, DMSO-d6): δ 9.91-9.78 (m, 1H), 8.15-7.99 (m, 1H), 7.23-7.08 (m, 1H), 3.97 (s, 3H).

Step 2

In a 3 L, 3-necked round bottom flask, Intermediate (1) (95 g, 669 mmol) was charged with dichloromethane (2 L) at 0° C. To this reaction mixture, boron tribromide (184 g, 736 mmol) was added dropwise. Then reaction mixture was allowed to stir at rt, and the reaction completion was monitored by TLC. The resultant mixture was poured into crushed ice and ammonium chloride solution. The slurry obtained was filtered through a pad of Celite. The organic phase was separated, and the aqueous layer was extracted several times with dichloromethane. The combined organic layer was dried over sodium sulfate, concentrated under reduced pressure, and crude mixture was purified by silica gel column chromatography using dichloromethane as mobile phase to obtain 69 g of 3-hydroxythiophene-2-carbaldehyde (2); Yield, 80.6%; ¹H NMR (400 MHz, DMSO-d6): δ 11.49 (s, 1H), 9.96-9.75 (m, 1H), 8.00-7.79 (m, 1H), 6.77-6.74 (m, 1H).

Step 3

In a 3-necked, 3 L round-bottom flask, Intermediate (2) (40 g, 312 mmol) was charged with 1,2-dichloroethane (2 L) under nitrogen. To this solution, methyl malonyl chloride (51.2 g, 375 mmol) was added dropwise, and reaction mixture was refluxed for 2 h followed by addition of TEA (47.3 g, 468 mmol) at rt. Reaction completion was monitored by TLC, and the mixture was cooled to rt and poured into water. The reaction mixture was extracted with dichloromethane. The organic layer was separated, dried over sodium sulfate, concentrated under reduced pressure, and purified by silica gel column chromatography (5-40% EtOAc in hexanes) to obtain 20.5 g of methyl 5-oxo-5H-thieno[3,2-b]pyran-6-carboxylate (3); Yield, 31.2%; ¹H NMR (400 MHz, DMSO-d6): δ 8.96 (s, 1H), 8.29 (d, J=5.5 Hz, 1H), 7.28 (d, J=5.5 Hz, 1H), 3.78 (s, 3H).

Step 4 Intermediate (3) (30 g, 142.8 mmol) was charged in a round-bottom flask, and was added concentrated H₂SO₄ (180 mL) at 0° C. followed by HNO₃ (90 mL) addition dropwise maintaining the temperature. Reaction completion was monitored by TLC. The resultant mixture was poured into ice-water slurry and the mixture was extracted with dichloromethane, and dried over sodium sulfate. The organic layer was concentrated under reduced pressure to obtain the crude compound, which was purified by silica gel column chromatography (dichloromethane gradient) to get 18.2 g of methyl-2-nitro-5-oxo-5H-thieno[3,2-b]pyran-6-carboxylate (4); Yield, 51.6%; ¹H NMR (400 MHz, DMSO-d6): δ 8.96 (s, 1H), 8.34 (s, 1H), 3.82 (s, 3H).

Step 5

Intermediate (4) (16 g, 62.7 mmol), acetic acid (240 mL), and iron powder (31.6 g, 564.3 mmol) were charged in a 500 mL single necked round-bottom flask. The reaction mixture was refluxed for 2 h and the reaction completion was monitored by TLC. Acetic acid was distilled off from crude reaction mixture. The resultant mixture was purified by column chromatography to afford 10.5 g of methyl 2-amino-5-oxo-5H-thieno[3,2-b]pyran-6-carboxylate (5); Yield, 74.4%; ¹H NMR (400 MHz, DMSO-d6): δ 8.38 (s, 1H), 8.18 (s, 2H), 5.99 (s, 1H), 3.73 (s, 3H).

In a similar manner, the following compounds were synthesized:

### Example 1

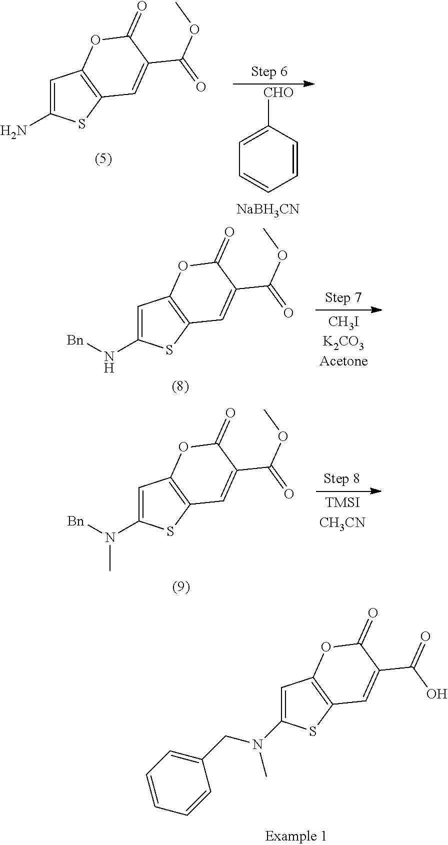

Step 6

Common Intermediate (5) (0.5 g, 2.22 mmol) from Scheme 5 was charged in 100 mL single necked round-bottomed flask along with benzaldehyde. Catalytic acetic acid was added to this reaction mixture and heated to 80° C. After starting was consumed, reaction mixture was brought to room temperature and EtOH was charged as solvent. Sodium cyanoborohydride (0.209 g, 3.33 mmol) was added at room temperature. On reaction completion, solvent was distilled off and quenched into water. Compound was extracted using dichloromethane and dried using anhydrous sodium sulphate. Organic layer was concentrated under reduced pressure and obtained compound was purified by column chromatography (Gradient 0-3% MeOH in DCM) to obtain 0.35 g of methyl 2-(benzylamino)-5-oxo-5H-thieno[3,2-b]pyran-6-carboxylate (8); Yield, 41.66%; ¹H NMR (400 MHz, DMSO-d₆): δ 9.2 (s, 1H), 8.46 (s, 1H), 7.31-7.39 (m, 5H), 6.2 (s, 1H), 4.4-4.47 (s, 2H), 3.65-3.68 (s, 3H).

Step 7

In 100 mL single necked round-bottomed flask, Intermediate (8) (0.3 g, 0.952 mmol) was charged in acetone. To this reaction mixture, CH₃I (0.268 g, 1.904 mmol), K₂CO₃ (0.26 g, 1.904 mmol) was added and reaction was refluxed for 1 h. Upon completion of reaction monitored by TLC, solvent was concentrated under reduced pressure to obtain crude compound. Crude compound was partitioned between water and dichloromethane. Organic layer was dried over anhydrous sodium sulfate to obtain 0.25 g of methyl 2-(benzyl(methyl)amino)-5-oxo-5H-thieno[3,2-b]pyran-6-carboxylate (9) which was used without any further purification; Yield, 79.9%; ¹H NMR (400 MHz, DMSO-d6): δ 8.54 (s, 1H), 7.29-7.41 (m, 5H), 6.41 (s, 1H), 4.74 (s, 2H), 3.69 (s, 2H and 3H), 3.18 (s, 3H).

Step 8

In a 50 mL single necked flask, Intermediate (9) (0.1 g, 0.303 mmol) was charged in CH₃CN (10 mL). TMSI (0.06 g, 0.303 mmol) was added and stirred under inert condition for 2 h. To this reaction mixture, water was added and allowed to stir for 15 min. Compound was extracted using dichloromethanedichloromethane. Organic phase was given saturated sodium thiosulphate and organic layer was concentrated under reduced pressure to obtained crude solid compound which was washed with 20% ethyl acetate in hexane and dried under vacuum to afford 50 mg of 2-(benzyl(methyl) amino)-5-oxo-5H-thieno[3,2-b]pyran-6-carboxylic acid (Example 1); Yield, 55.6%; ¹H NMR (400 MHz, DMSO-d₆): δ 12.18 (s, 1H), 8.57 (s, 1H), 7.30-7.41 (m, 5H), 6.53 (s, 1H), 4.77 (s, 2H), 3.2 (s, 3H), MS(ESI) 329.9 (M+14); HPLC, 98%.

### Example 2

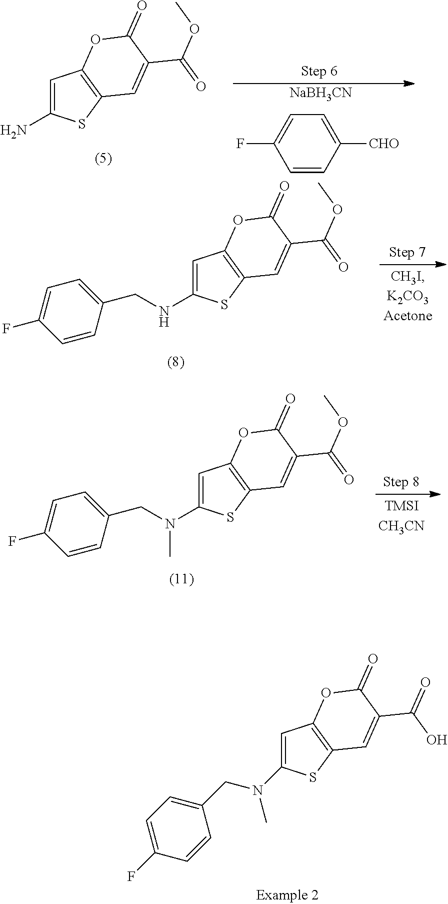

Step 6

Common Intermediate (5) (0.7 g, 3.11 mmol) from Scheme 5 was charged in 100 mL single necked round-bottomed flask along with 4-fluorobenzaldehyde (0.385 g, 3.11 mmol). Catalytic acetic acid was added to this reaction mixture and heated to 80° C. After starting was consumed, reaction mixture was brought to room temperature and EtOH was charged as solvent. Sodium cyanoborohydride (0.292 g, 34.66 mmol) was added at room temperature. On reaction completion, solvent was distilled off and quenched into water. Compound was extracted using dichloromethane and dried using anhydrous sodium sulphate. Organic layer was concentrated under reduced pressure and obtained compound was purified by column chromatography (Gradient 0-3% MeOH in DCM) to obtain 0.5 g of methyl 2-(4-fluorobenzylamino)-5-oxo-5H-thieno[3,2-b]pyran-6-carboxylate (10); Yield, 50%.

Step 7

In 100 mL single necked round-bottomed flask, Intermediate (10) (0.5 g, 1.501 mmol) was charged in acetone. To this reaction mixture, CH₃I (0.42 g, 13 mmol), K₂CO₃ (0.41 g, 3 mmol) was added and reaction was refluxed for 1 h. Upon completion of reaction monitored by TLC, solvent was concentrated under reduced pressure to obtain crude compound. Crude compound was partitioned between water and dichloromethane. Organic layer was dried over anhydrous sodium sulfate to obtain 0.35 g of methyl 2-((4-fluorobenzyl)(methyl)amino)-5-oxo-5H-thieno[3,2-b]pyran-6-carboxylate (11); Yield, 67.3%, which was used without any further purification.

Step 8

In a 50 mL single necked flask, Intermediate (11) (0.1 g, 0.288 mmol) was charged in CH₃CN (10 mL). TMSI (0.057 g, 0.288 mmol) was added and stirred under inert condition for 2 h. To this reaction mixture, water was added and allowed to stir for 15 min. Compound was extracted using dichloromethane. Organic phase was given saturated sodium thiosulphate and organic layer was concentrated under reduced pressure to obtained crude solid compound which was washed with 20% ethyl acetate in hexane and dried under vacuum to afford 50 mg of 24(4-fluorobenzyl)(methyl)amino)-5-oxo-5H-thieno[3,2-b]pyran-6-carboxylic acid (Example 2); Yield, 55.55%; ¹H NMR (400 MHz, DMSO-d₆): δ 12.19 (s, 1H), 8.58 (s, 1H), 7.20-7.38 (m, 4H), 6.54 (s, 1H), 4.75 (s, 2H), 3.18 (s, 3H), MS(ESI) 347.7 (M+14); HPLC, 97%.

### Example 3

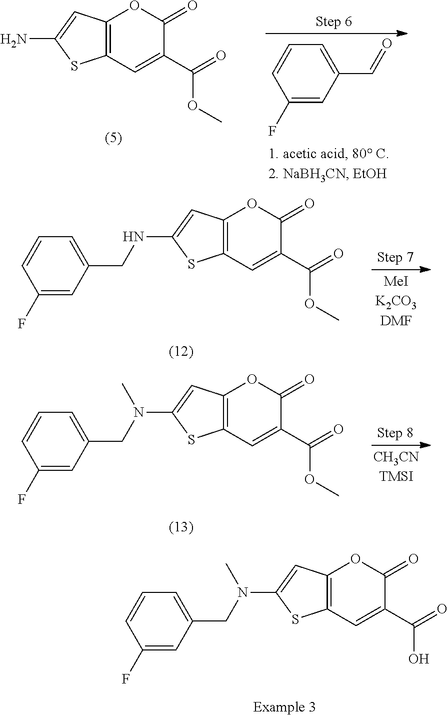 [SMILES: COC(=O)c1cc2sc(N)cc2oc1=O ; O=Cc1cccc(F)c1 ; COC(=O)c1cc2sc(NCc3cccc(F)c3)cc2oc1=O ; COC(=O)c1cc2sc(N(C)Cc3cccc(F)c3)cc2oc1=O ; CN(Cc1cccc(F)c1)c1cc2oc(=O)c(C(=O)O)cc2s1]

Step 6

To a mixture of Common Intermediate (5) (600 mg, 2.666 mmol) from Scheme 5 and 3-fluorobenzaldehyde (496 mg, 4.0 mmol) was added HOAc (150 uL). The resultant mixture was heated under microwave conditions at 80° C. for 1 h. To the reaction mixture was added EtOH (15 mL) and sodium cyanoborohydride (1.85 g, 29.33 mmol) at rt. The resultant mixture was stirred overnight, and mixture was concentrated. To the product was added water and the mixture was extracted with dichloromethane, and the organic layer was separated. The organic phase was dried over anhydrous sodium sulfate and concentrated under reduced pressure. The crude product was purified by silica gel column chromatography to afford 240 mg of methyl 2-[(3-fluorobenzyl)amino]-5-oxo-5H-thieno[3,2-b]pyran-6-carboxylate (12); Yield, 27.0%; ¹H NMR (400 MHz, DMSO-d6): δ 9.14 (s, 1H), 8.45 (s, 1H), 7.46-7.36 (m, 1H), 7.19 (d, J=6.9 Hz, 2H), 7.12 (t, J=7.9 Hz, 1H), 6.19 (s, 1H), 4.48 (s, 2H), 3.66 (s, 3H).

Step 7

To the suspension of Intermediate (12) (340 mg, 1.021 mmol) in DMF (30 mL) was added potassium carbonate (282 mg, 2.042 mmol) followed by methyl iodide (1.45 g, 10.21 mmol) at rt. The resultant mixture was heated to 60° C. for 3 h. The reaction was monitored by TLC and the reaction mixture was concentrated under reduced pressure. The crude product was purified by silica gel column chromatography to afford product 300 mg of methyl 2-((3-fluorobenzyl)(methyl)amino)-5-oxo-5H-thieno[3,2-b]pyran-6-carboxylate (13); Yield, 84.7%; ¹H NMR (400 MHz, DMSO-d6): δ 8.53 (s, 1H), 7.42-7.40 (m, 1H), 7.13-7.09 (m, 3H), 6.37 (s, 1H), 4.74 (s, 2H), 3.68 (s, 3H), 3.18 (s, 3H).

Step 8

In a 100 mL single necked flask, Intermediate (13) (300 mg, 0.864 mmol) was dissolved with dry acetonitrile (50 mL). To the solution was added trimethylsilyl iodide (346 mg, 1.73 mmol) at 20° C., and the mixture was stirred for 16 h at that temperature. After the reaction completion, the mixture was filtered, and the cake was washed with a small amount of acetonitrile to afford 150 mg of 2-[(3-fluorobenzyl)(methyl)amino]-5-oxo-5H-thieno[3,2-b]pyran-6-carboxylic acid (Example 3); Yield, 52.1%; ¹H NMR (400 MHz, DMSO-d6): δ 8.56 (s, 1H), 7.42-41 (m, 1H), 7.18-7.07 (m, 3H), 6.49 (s, 1H), 4.76 (s, 2H), 3.20 (s, 3H); ¹⁹F NMR (376 MHz, DMSO-d6): δ −112.63; LC-MS (ESI): 334 (M+H); HPLC: 98.5%.

### Example 4

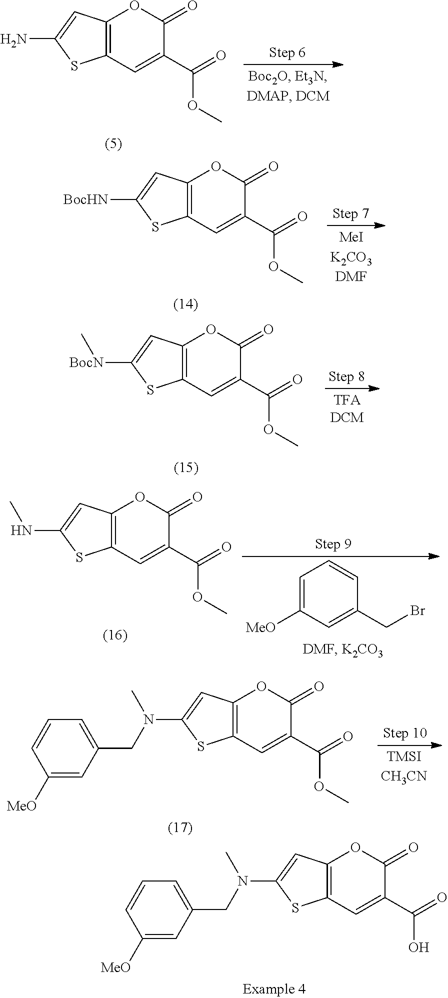

Step 6

To a solution of Common Intermediate (5) (4.5 g, 20.0 mmol) from Scheme 5 and di-tert-butyldicarbonate (8.73 g, 40.0 mmol) in dichloromethane (60 mL) was added triethylamine (4.05 g, 40.0 mmol) and N,N′-dimethylaminopyridine (366 mg, 3 mmol) at rt. The resultant mixture was stirred for 4 h at rt, concentrated in vacuo on reaction completion, and the crude product was purified by silica gel column chromatography to afford 3.0 g of methyl 2-((tert-butoxycarbonyl)amino)-5-oxo-5H-thieno-[3,2-b]pyran-6-carboxylate (14); Yield: 46.1%; ¹H NMR (400 MHz, DMSO-d6): δ 11.58 (s, 1H), 8.78 (s, 1H), 6.54 (s, 1H), 3.73 (s, 3H), 1.48 (s, 9H).

Step 7

To a solution of Intermediate (14) (3.0 g, 9.23 mmol) and potassium carbonate (2.55 g, 18.46 mmol) in DMF (60 mL) was added methyl iodide (13.1 g, 92.31 mmol) at rt. The resultant mixture was heated to 60° C. under N₂ for 4 h. After the reaction completion, the mixture was concentrated in vacuo, and the crude product was diluted with water (50 mL) followed by extraction several times with EtOAc. The combined organic layer was dried over anhydrous sodium sulfate and concentrated in vacuo to afford 3.5 g of methyl 2-((tert-butoxycarbonyl)-(methyl)amino)-5-oxo-5H-thieno[3,2-b]pyran-6-carboxylate (15) which was used for next step without further purification; ¹H NMR (400 MHz, DMSO-d6): δ 8.78 (s, 1H), 6.86 (s, 1H), 3.74 (s, 3H), 3.39 (s, 3H), 1.52 (s, 9H).

Step 8

To a solution of Intermediate (15) (3.5 g, 9.23 mmol) in dichloromethane (30 mL) was added trifluoroacetic acid (10 mL) at rt and stirred for 16 h. On completion, the reaction mixture was concentrated in vacuo, and the crude product was triturated with diethyl ether and filtered to afford 2.0 g of methyl 2-(methylamino)-5-oxo-5H-thieno[3,2-b]pyran-6-carboxylate (16) as yellow solid; Yield, 90.9% (for steps 13 and 14); ¹H NMR (400 MHz, DMSO-d6): δ 8.43 (s, 1H) 6.08 (s, 1H), 3.66 (s, 3H), 3.40 (s, 3H).

Step 9

To a solution of Intermediate (16) (400 mg, 1.67 mmol) and potassium carbonate (461 mg, 3.34 mmol) in DMF (5 mL) was added 1-(bromomethyl)-3-methoxybenzene (1.34 g, 6.69 mmol) at rt, and the mixture was heated under N₂ for 4 h at 60° C. After the reaction completion, the mixture was concentrated in vacuo, and the crude product was dissolved in water (50 mL) and extracted several times with EtOAc. The combined organic layer was dried over anhydrous sodium sulfate, concentrated in vacuo, and purified by silica gel column chromatography to afford 500 mg of methyl 2-((3-methoxybenzyl)(methyl)amino)-5-oxo-5H-thieno[3,2-b]pyran carboxylate (17) as yellow solid; Yield: 83.3%; ¹H NMR (400 MHz, DMSO-d6): δ 8.51 (s, 1H), 7.28 (t, J=8.1 Hz, 1H), 8.88-8.84 (m, 3H), 6.36 (s, 1H), 4.68 (s, 2H), 3.72 (s, 3H), 3.67 (s, 3H), 3.15 (s, 3H).

Step 10

In a 100 mL single necked flask, Intermediate (17) (400 mg, 1.114 mmol) was dissolved in dry acetonitrile (50 mL). To the solution was added trimethylsilyl iodide (346 mg, 1.66 mmol) at rt, and the mixture was stirred for 16 h. After the reaction completion, the mixture was filtered, and the crude product was washed with acetonitrile and dried in vacuo to afford 190 mg of 2-((3-methoxybenzil)-(methyl)amino)-5-oxo-5H-thieno[3,2-b]pyran-6-carboxylic acid (Example 4); Yield: 49.5%; ¹H NMR (400 MHz, DMSO-d6): δ 12.16 (s, 1H), 8.55 (s, 1H), 7.28 (t, J=8.0 Hz, 1H), 6.91-6.81 (m, 3H), 6.49 (s, 1H), 4.71 (s, 2H), 3.73 (s, 3H), 3.17 (s, 3H), LC-MS (ESI): 346 (M+H)⁺; HPLC: 98.8%.

### Example 5

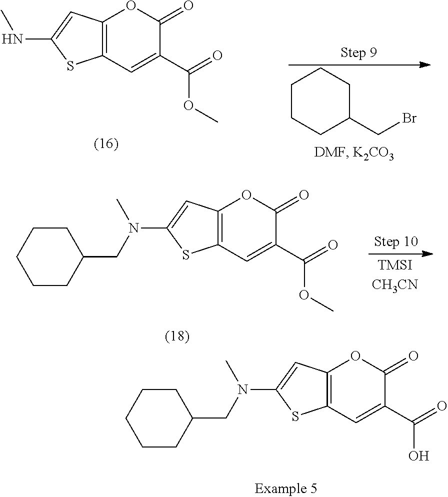

Step 9

To a solution of Intermediate (16) (400 mg, 1.67 mmol) from Example 4 and potassium carbonate (691 mg, 5.01 mmol) in DMF (10 mL) was added (bromomethyl)cyclohexane (1.18 g, 6.68 mmol) at rt and heated at 60° C. under N₂ for 4 h. After the reaction completion, the solution was concentrated, and the crude product was treated with water (50 mL) and extracted several times with EtOAc. The combined organic layer was dried over anhydrous sodium sulfate and concentrated in vacuo. The crude product was purified by silica gel column chromatography to afford 470 mg of methyl 2-((cyclohexylmethyl)(methyl)amino)-5-oxo-5H-thieno[3,2-b]pyran-6-carboxylate (18), Yield: 83.9%; ¹H NMR (400 MHz, DMSO-d6): δ 8.46 (s, 1H), 6.26 (s, 1H), 3.67 (s, 3H), 3.28 (d, J=4.8 Hz, 2H), 3.10 (s, 3H), 1.79-1.59 (m, 6H), 1.21-1.08 (m, 3H), 1.00-0.93 (m, 2H).

Step 10

In a 100 mL single necked flask, Intermediate (18) (400 mg, 1.194 mmol) was dissolved in dry acetonitrile (50 mL). To the solution was added trimethylsilyl iodide (477.6 mg, 2.388 mmol) at rt and the mixture was stirred for 16 h at rt. After the reaction completion, the solution was concentrated, and the product was purified by silica gel column chromatography to afford 240 mg of 2-((cyclohexylmethyl)(methyl)amino)-5-oxo-5H-thieno[3,2-b]pyran-6-carboxylic acid (Example 5); Yield: 62.5%; ¹H NMR (400 MHz, CDCl₃): δ 12.28 (s, 1H), 8.42 (s, 1H), 5.96 (s, 1H), 3.28 (d, J=7.4 Hz, 2H), 3.17 (s, 3H), 1.82-1.69 (m, 6H), 1.32-1.15 (m, 3H), 1.04-0.95 (m, 2H), LC-MS (ESI): 322 (M+H)⁺; HPLC: 98.1%.

### Example 6

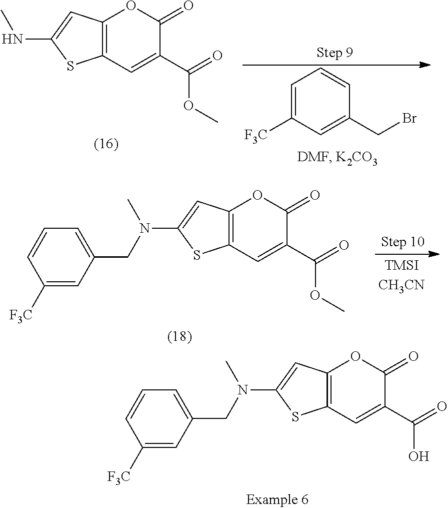

Step 9

To a solution of Intermediate (16) (380 mg, 1.59 mmol) from Example 4 and potassium carbonate (439 mg, 3.18 mmol) in DMF (10 mL) was added 1-(bromomethyl)-3-(trifluoromethyl)benzene (1.14 g, 4.77 mmol) at rt, and the mixture was heated for 4 h at 60° C. After the reaction completion, the mixture was concentrated in vacuo, and the crude product was treated with water (50 mL) followed by extraction several times with EtOAc. The combined organic layer was dried over anhydrous sodium sulfate and concentrated. The crude product was purified by silica gel column chromatography to afford 550 mg of methyl 2-(methyl(3-(trifluoromethyl)benzyl)amino)-5-oxo-5H-thieno[3,2-b]pyran-6-carboxylate (19); Yield: 87.1%; ¹H NMR (400 MHz, DMSO-d6): δ 8.54 (s, 1H), 7.69-7.55 (m, 4H), 6.41 (s, 1H), 4.83 (s, 2H), 3.67 (s, 3H), 3.18 (s, 3H).

Step 10

In a 50 mL single necked flask, Intermediate (19) (250 mg, 0.63 mmol) was dissolved in dichloromethane (8 mL). To the solution was added trimethylsilyl iodide (504 mg, 2.52 mmol) at rt, and the mixture was stirred for 1.5 h at rt. After the reaction completion, diethyl ether (30 mL) was added, and the mixture was concentrated. To the crude solid was added 25 mL of solvent mixture (methanol:dichloromethane; 20:1), and the mixture was stirred for 30 min. The resultant suspension was filtered, and the filter cake was washed with a small amount of methanol to afford 190 mg of 2-(methyl(3-(trifluoromethyl)benzyl)amino)-5-oxo-5H-thieno[3,2-b]pyran-6-carboxylic acid (Example 6) as yellow solid; Yield: 78.8%; ¹H NMR (400 MHz, DMSO-d6): δ 12.15 (s, 1H), 8.57 (s, 1H), 7.69-7.56 (m, 4H), 6.52 (s, 1H), 4.85 (s, 2H), 3.21 (s, 3H); ¹⁹F NMR (376 MHz, DMSO-d6): δ −61.08; LC-MS (ESI): 384 (M+H)⁺; HPLC: 99.6%.

### Example 7

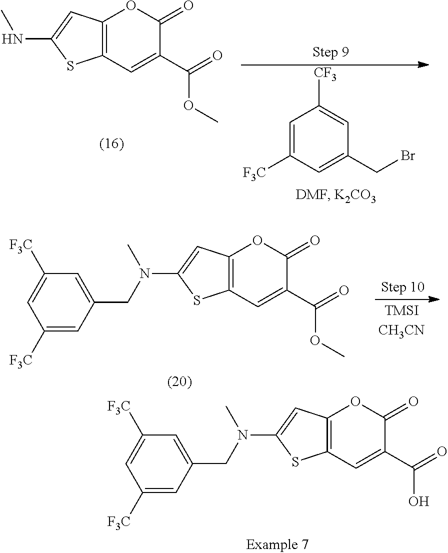

Step 9

To a solution of Intermediate (16) (380 mg, 1.59 mmol) and potassium carbonate (439 mg, 3.18 mmol) in DMF (10 mL) was added 1-(bromomethyl)-3,5-bis(trifluoromethyl)benzene (1.46 g, 4.77 mmol) at rt, and the mixture was heated to 60° C. under N₂ for 4 h. After the reaction completion, the mixture was concentrated, and the crude product was treated with water followed by extraction several times with EtOAc. The combined organic layer was dried over anhydrous sodium sulfate and concentrated in vacuo. The resultant product was purified by silica gel column chromatography to afford 500 mg of the desired product, 2-((3,5-bis(trifluoromethyl)benzyl)(methyl)amino)-5-oxo-5H-thieno[3,2-b]pyran-6-carboxylate (20) as yellow solid; Yield: 67.6%; ¹H NMR (400 MHz, DMSO-d6): δ 8.56 (s, 1H), 8.07 (s, 1H), 7.97 (s, 1H), 6.44 (s, 1H), 4.91 (s, 2H), 3.68 (s, 3H), 3.22 (s, 3H).

Step 10

In a 50 mL single necked flask, Intermediate (20) (250 mg, 0.54 mmol) was dissolved in dichloromethane (8 mL). To the solution was added trimethylsilyl iodide (424 mg, 2.12 mmol) at rt, and the resultant mixture was stirred for 1.5 h. After the reaction completion, diethyl ether (30 mL) was added to the mixture and concentrated in vacuo. To the crude solid product was added a solvent mixture containing dichloromethane and methanol (20:1). The mixture was stirred for 30 min and the suspension was filtered. The filter cake was washed with a small amount of methanol to afford 200 mg of the desired compound, 2-((3,5-bis(trifluoromethyl)benzyl) (methyl)amino)-5-oxo-5H-thieno-[3,2-b]pyran-6-carboxylic acid (Example 7) as a yellow solid; Yield: 82.3%; ¹H NMR (400 MHz, DMSO-d6): δ 12.18 (s, 1H), 8.60 (s, 1H), 8.08 (s, 1H), 7.98 (s, 1H), 6.55 (s, 1H), 4.94 (s, 2H), 3.24 (s, 3H); ¹⁹F NMR (376 MHz, DMSO-d6): δ −61.29; LC-MS (m/z, ESI): 452 (M+H)⁺; HPLC: 98.2%.

### Example 8

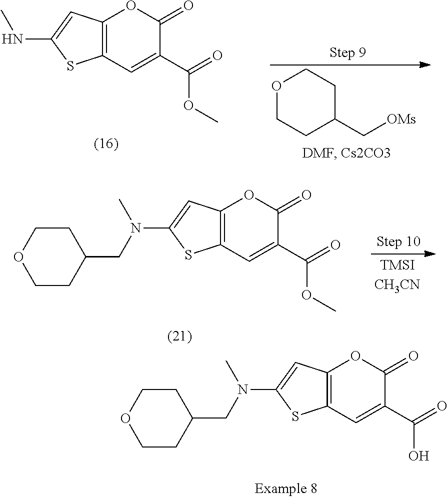

Step 9

To a solution of Intermediate (16) (700 mg, 2.93 mmol) from Example 4 and cesium carbonate (1.91 g, 5.86 mmol) in DMF (10 mL) was added tetrahydro-2H-pyran-4-yl)methyl methanesulfonate (2.84 g, 14.6 mmol) at rt, and the resultant mixture was heated to 100° C. under microwave conditions for 5 h. After the reaction completion, the reaction mixture was concentrated in vacuo, and the crude product obtained was dissolved in water (50 mL) and extracted several times with EtOAc. The combined organic layer was dried over anhydrous sodium sulfate and concentrated in vacuo. The crude product was purified by silica gel column chromatography to afford 550 mg of the desired methyl 2-(methyl-((tetrahydro-2H-pyran yl)methyl)amino)-5-oxo-5H-thieno[3,2-b]pyran-6-carboxylate (21) as yellow solid; Yield: 55.7%; ¹H NMR (400 MHz, CDCl₃): δ 8.33 (s, 1H), 5.84 (s, 1H), 4.00 (d, J=8.1 Hz, 2H), 3.88 (s, 3H), 3.37 (t, J=11.8 Hz, 2H), 3.29 (d, J=7.3 Hz, 2H), 3.14 (s, 3H), 2.06 (s, 2H), 1.43-1.38 (m, 3H).

Step 10

In a 100 mL single necked flask, Intermediate (21) (450 mg, 1.34 mmol) was dissolved in dry acetonitrile (50 mL). To the solution was added trimethylsilyl iodide (534 mg, 2.67 mmol) at rt, and the mixture was stirred for 16 h. After the reaction completion, the mixture was concentrated in vacuo and purified by prep-HPLC to afford 90 mg of the desired product, 2-(methyl((tetrahydro-2H-pyran-4-yl)-methyl)amino)-5-oxo-5H-thieno[3,2-b]pyran-6-carboxylic acid (Example 8); Yield: 20.1%; ¹H NMR (400 MHz, CDCl₃): δ 12.24 (s, 1H), 8.46 (s, 1H), 5.98 (s, 1H), 4.00 (d, J=8.1 Hz, 2H), 3.41-3.34 (m, 4H), 3.20 (s, 3H), 1.62-1.57 (m, 6H), 1.43-1.40 (m, 3H); LC-MS (ESI): 324 (M+H)⁺; HPLC: 98.5%.

### Example 9

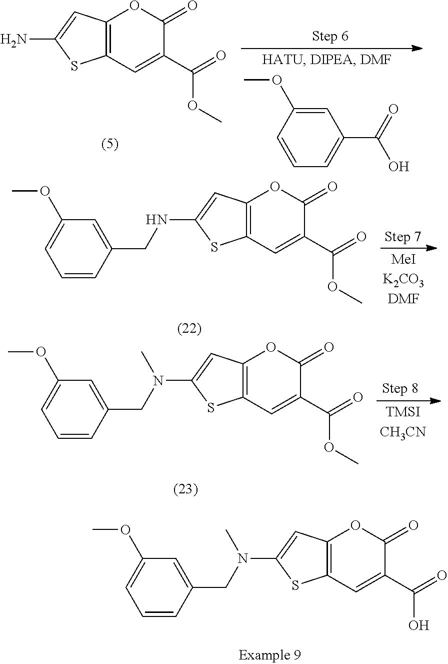

Step 6

To a solution of Common Intermediate (5) (400 mg, 1.78 mmol, 1.0 eq), 3-methoxybenzoic acid (325 mg, 2.14 mmol, 1.2 eq) and HATU (1.01 g, 2.67 mmol, 1.5 eq) in DMF (15 mL), was added diisopropylethyl amine (689 mg, 5.34 mmol, 3.0 eq) at rt. The reaction mixture was stirred at rt for 16 h. The reaction was monitored by TLC. The mixture was concentrated in vacuo. The residue was purified by silica gel column chromatography to afford 398 mg of methyl 2-(3-methoxybenzamido)-5-oxo-5H-thieno[3,2-b]pyran-6-carboxylate (22), Yield: 62.5%; ¹H NMR (400 MHz, DMSO-d6): δ 12.37 (s, 1H), 8.86 (s, 1H), 7.61-7.51 (m, 3H), 7.24 (d, J=8 Hz, 1H), 6.95 (s, 1H), 3.84 (s, 3H), 3.75 (s, 3H).

Step 7

To a solution of Intermediate (22) (300 mg, 0.84 mmol, 1.0 eq) and K₂CO₃ (232 mg, 1.68 mmol, 2.0 eq) in DMF (10 mL) was added iodomethane (1.19 g, 8.4 mmol, 10 eq) at rt. Then the mixture was heated to 70° C. under N₂ and stirred for 2 h at that temperature. The mixture was concentrated. The residue was dissolved with water (50 mL) and the solution was extracted with ethyl acetate. The combined organic layer was dried over anhydrous sodium sulfate, concentrated in vacuo, and the residue was purified by silica gel column chromatography to afford 274 mg of the product methyl 2-(3-methoxy-N-methylbenzamido)-5-oxo-5H-thieno[3,2-b]pyran-6-carboxylate (23), yield: 87.5%; ¹H NMR (400 MHz, DMSO-d6): δ 8.88 (s, 1H), 7.44 (t, J=8.2 Hz, 1H), 7.19-7.11 (m, 4H), 3.80 (s, 3H), 3.76 (s, 3H), 3.48 (s, 3H).

Step 8

In a 250 mL single necked flask, Intermediate (23) (210 mg, 0.56 mmol, 1.0 eq) was dissolved in dry dichloromethane (14 mL). To the solution was added trimethylsilyl iodide (280 mg, 1.40 mmol, 2.5 eq) at rt, and the resultant mixture was stirred for 16 h at rt. The mixture was concentrated, filtered, and the crude product was triturated with methanol to afford 150 mg of the product 2-(3-methoxy-N-methylbenzamido)-5-oxo-5H-thieno[3,2-b]pyran-6-carboxylic acid (Example 9), Yield: 74.6%; ¹H NMR (400 MHz, DMSO-d6): δ 12.64 (s, 1H), 8.87 (s, 1H), 7.74 (t, J=8 Hz, 1H), 7.20-7.12 (m, 4H), 3.80 (s, 3H), 3.48 (s, 3H); LC-MS (ESI): 360 (M+H), 382 (M+23); HPLC: 97.2%.

### Example 10

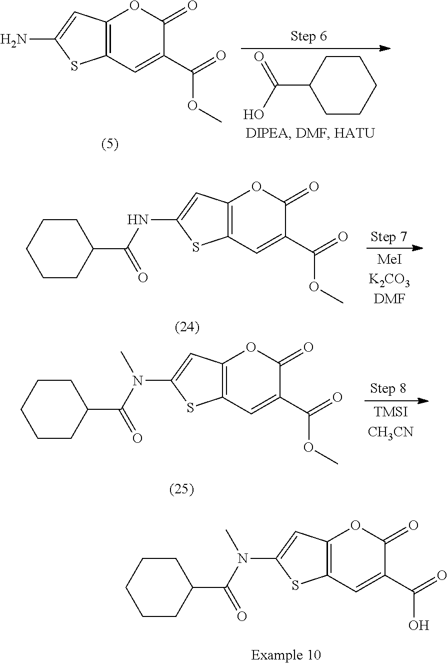

Step 6

To a solution of Common Intermediate (5) (950 mg, 4.22 mmol), cyclohexanecarboxylic acid (648 mg, 5.07 mmol) and HATU (2.41 g, 6.33 mmol) in DMF (25 mL) was added diisopropylethylamine (1.63 g, 12.7 mmol), and the reaction mixture was stirred for 16 h at rt. On reaction completion, the mixture was concentrated in vacuo, added water (50 mL) and stirred for 30 min at rt. The resultant suspension was filtered, and the filter cake was washed with diethyl ether to afford 800 mg of the desired product, methyl 2-(cyclohexanecarboxamido)-5-oxo-5H-thieno[3,2-b]pyran-6-carboxylate (24) as a yellow solid; Yield: 56.5%; ¹H NMR (400 MHz, DMSO-d6): δ 12.00 (s, 1H), 8.80 (s, 1H), 6.70 (s, 1H), 3.74 (s, 3H), 2.46-2.41 (m, 1H), 1.84-1.81 (m, 4H), 1.75-1.72 (m, 1H), 1.41-1.38 (m, 2H), 1.21-1.14 (m, 3H).

Step 7

To a solution of Intermediate (24) (500 mg, 1.49 mmol) and potassium carbonate (621 mg, 4.47 mmol) in DMF (20 mL) was added methyl iodide (3.18 g, 22.3 mmol) at rt, and the resultant mixture was heated at 70° C. under N₂ atm for 4 h. After the reaction completion, the solution mixture was concentrated in vacuo and the crude product obtained was dissolved in water (20 mL). The reaction mixture was extracted several times with EtOAc, and the combined organic layer was dried over anhydrous sodium sulfate. The crude mixture was concentrated in vacuo and purified by silica gel column chromatography to afford 330 mg of the desired product, methyl 2-(N-methylcyclohexanecarboxamido)-5-oxo-5H-thieno[3,2-b]-pyran-6-carboxylate (25) as a yellow solid; Yield: 63.4%; ¹H NMR (400 MHz, DMSO-d6): δ, 8.80 (s, 1H), 7.00 (s, 1H), 3.74 (s, 3H), 3.57 (s, 3H), 2.97-2.94 (m, 1H), 1.81-1.64 (m, 4H), 1.37-1.33 (m, 4H), 1.20-1.14 (m, 2H).

Step 8

In a 100 mL single necked flask, Intermediate (25) (340 mg, 0.97 mmol) was dissolved in dry acetonitrile (50 mL). To the solution was added trimethylsilyl iodide (389 mg, 1.95 mmol) at rt, and the mixture was stirred for 16 h at rt. After the reaction completion, the mixture was filtered and the filter cake was washed with acetonitrile to afford 300 mg of the desired product, 2-(N-methylcyclohexane-carboxamido)-5-oxo-5H-thieno[3,2-b]pyran-6-carboxylic acid (Example 10); Yield: 91.9%; ¹H NMR (400 MHz, DMSO-d6): δ 12.58 (s, 1H), 8.81 (s, 1H), 7.03 (s, 1H), 3.58 (s, 3H), 2.97 (s, 1H), 1.72-1.20 (m, 10H); LC-MS (ESI): 336 (M+H)⁺; HPLC: 99.6%.

### Example 11

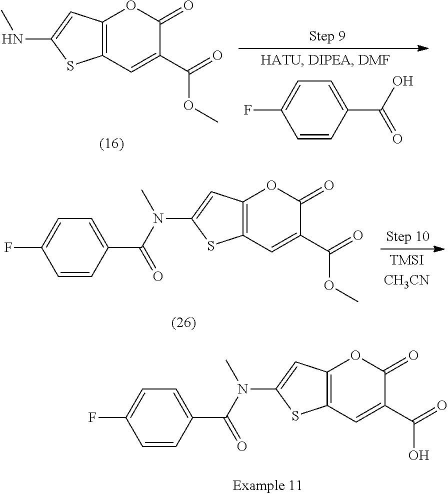

Step 9

To a solution of Intermediate (16) (900 mg, 4.0 mmol) from Example 4, 4-fluorobenzoic acid (672 mg, 4.8 mmol) and (1-[Bis(dimethylamino)methylene]-1H-1,2,3-triazolo[4,5-b]pyridinium-3-oxid-hexafluoro-phosphate) (2.28 g, 6.0 mmol) in DMF (30 mL), was added N,N′-diisopropylethylamine (1.55 g, 12.0 mmol) at rt, and the reaction mixture was stirred for 16 h at rt. The reaction was monitored by TLC. On reaction completion, the reaction mixture was concentrated in vacuo, and treated with water (50 mL), and the resultant mixture was stirred for 30 min at rt. The mixture was filtered and the yellow solid was washed with diethyl ether to afford 890 mg of methyl 2-(4-fluorobenzamido)-5-oxo-5H-thieno[3,2-b]pyran-6-carboxylate (26); Yield, 64.1%; ¹H NMR (400 MHz, DMSO-d6): δ 12.41 (s, 1H), 8.85 (s, 1H), 8.12-8.08 (m, 2H), 7.44 (t, J=8.2 Hz, 2H), 6.93 (s, 1H), 3.75 (s, 3H).

Step 10

In a 50 mL single necked flask, Intermediate (26) (300 mg, 0.831 mmol) was dissolved in dry acetonitrile (30 mL). To the solution was added trimethylsilyl iodide (346 mg, 1.66 mmol) at rt, and the mixture was stirred for 16 h at rt. After the reaction completion, the mixture was filtered, and the crude product was washed with acetonitrile and dried in vacuo to afford 200 mg of 2-(4-fluoro-N-methylbenzamido)-5-oxo-5H-thieno[3,2-b]pyran-6-carboxylic acid (Example 11); Yield, 69.4%; ¹H NMR (400 MHz, DMSO-d6): δ 12.65 (s, 1H), 8.87 (s, 1H), 7.74 (t, J=8 Hz, 2H), 7.37 (t, J=8 Hz, 2H), 7.14 (s, 1H), 3.49 (s, 3H), ¹⁹F NMR (376 MHz, DMSO-d6): δ −108.66; LC-MS (ESI): 348 (M+H); HPLC: 97.2%.

### Example 12

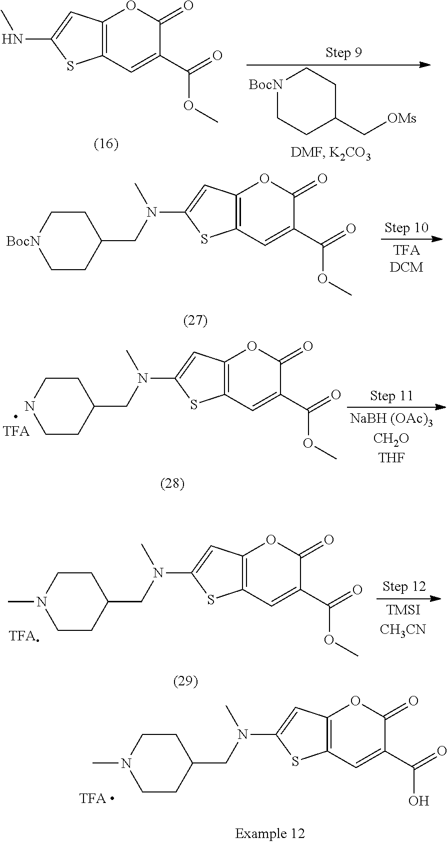

Step 9

To a solution of Intermediate (16) (800 mg, 3.35 mmol) from Example 4 and cesium carbonate (2.18 g, 6.69 mmol) in DMF (50 mL) was added tert-butyl 4-[{(methylsulfonyl)oxy}methyl]piperidine-1-carboxylate (4.90 g, 16.7 mmol) at rt followed by heating at 100° C. under N₂ atmosphere for 16 h. After the reaction completion, the mixture was concentrated in vacuo. The crude product was dissolved in water (30 mL) and extracted several times with EtOAc. The combined organic layer was dried over anhydrous sodium sulfate and concentrated in vacuo. The resultant crude product was purified by silica gel column chromatography to afford 1.4 g of tert-butyl4-(((6-(methoxy-carbonyl)-5-oxo-5H-thieno[3,2-b]pyran-2-yl)(methyl)amino)methyl)piperidine-1-carboxylate (27); Yield, 94.5%; ¹H NMR (400 MHz, DMSO-d6): δ 8.48 (s, 1H), 6.32 (s, 1H), 4.44 (s, 1H), 3.92 (d, J=8.2 Hz, 2H), 3.67 (s, 3H), 3.36 (d, J=7.0 Hz, 2H), 3.22-3.20 (m, 3H), 3.11 (s, 3H), 1.57 (t, J=14.8 Hz, 3H), 1.37 (s, 9H), 1.15-1.04 (m, 1H), 0.95 (dd, J=12.4, 8.6 Hz, 2H).

Step 10

To a solution of Intermediate (27) (1.1 g, 2.52 mmol) in dichloromethane (20 mL) was added TFA (7 mL) at rt, and the mixture was stirred under N₂ atm for 2 h. After the reaction completion, the mixture was concentrated in vacuo, and the crude product obtained was triturated with diethyl ether to afford 570 mg of the desired product, methyl 2-(methyl-(piperidin-4-ylmethyl)amino)-5-oxo-5H-thieno[3,2-b]pyran-6-carboxylate (28); Yield: 67.1%; ¹H NMR (400 MHz, DMSO-d6): δ 8.51 (s, 2H), 8.19 (s, 1H), 6.33 (s, 1H), 3.68 (s, 3H), 3.40 (d, J=7.1 Hz, 2H), 3.12 (s, 3H), 3.26-3.24 (m, 2H), 2.83-281 (m, 2H), 2.08-2.06 (m, 1H), 1.75 (d, J=12.9 Hz, 2H), 1.34-1.31 (m, 2H).

Step 11

To a solution of Intermediate (28) (600 mg, 1.79 mmol) in THF (40 mL) was added paraformaldehyde (269 mg, 8.95 mmol) and sodium triacetoxyborohydride (759 mg, 3.58 mmol) at rt and stirred for 1 h. After the reaction completion, the mixture was poured into dichloromethane (˜200 mL) to get a clear solution. This solution was dried over anhydrous sodium sulfate and concentrated in vacuo. The crude product was purified by silica gel column chromatography to afford 590 mg of the desired product, methyl 2-(methyl((1-methylpiperidin-4-yl)methyl)amino)-5-oxo-5H-thieno-[3,2-b]pyran-6-carboxylate (29); Yield: 94.1%; ¹H NMR (400 MHz, DMSO-d6): δ 8.50 (s, 1H), 6.32 (s, 1H), 3.67 (s, 3H), 3.37 (dd, J=17.9, 7.1 Hz, 5H), 3.17-3.09 (m, 5H), 1.97-1.89 (m, 1H), 1.70 (d, J=11.9 Hz, 2H), 1.49-1.27 (m, 2H), 1.21-1.13 (m, 2H).

Step 12

In a 50 mL single necked flask, Intermediate (29) (500 mg, 1.43 mmol) was dissolved in dry acetonitrile (25 mL). To the solution was added trimethylsilyl iodide (1.72 g, 8.58 mmol) at rt, and the mixture was stirred for 1.5 h at rt. After the reaction completion, water (1 mL) and diethyl ether (100 mL) was added into the reaction mixture, and the suspension was filtered. The resultant solid product was purified by prep-HPLC to afford 110 mg of the desired product, 2-(methyl((1-methylpiperidin-4-yl)methyl)amino)-5-oxo-5H-thieno[3,2-b]pyran-6-carboxylic acid (Example 12) as yellow solid; Yield: 23.0%; ¹H NMR (400 MHz, DMSO-d6): δ 12.15 (s, 1H), 9.25 (s, 1H), 8.51 (s, 1H), 6.46 (s, 1H), 3.43-3.40 (m, 4H), 3.15 (s, 3H), 2.88-2.85 (m, 2H), 2.72 (s, 3H), 2.00 (s, 1H), 1.82 (d, J=13.7 Hz, 2H), 1.42-1.39 (m, 2H); ¹⁹F NMR (376 MHz, DMSO-d6): δ −73.50; LC-MS (ESI): 337 (M+H)⁺; HPLC: 97.3%.

### Example 13

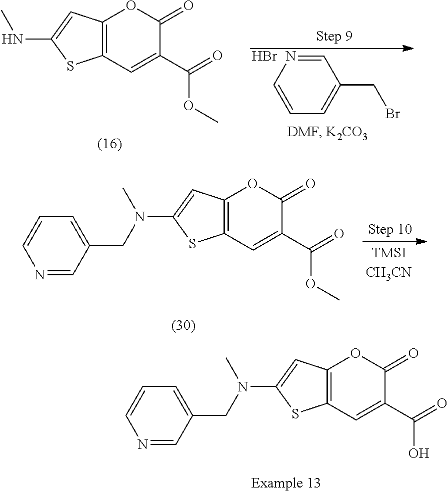

Step 9

To a solution of Intermediate (16) (700 mg, 2.93 mmol) from Example 4 and K₂CO₃ (809 mg, 5.86 mmol) in DMF (80 mL) was added hydrobromic acid salt of 3-(bromomethyl)pyridine (1.11 g, 4.39 mmol) at rt, and the reaction mixture was stirred for 5 h at 70° C. under N₂ atm. After the reaction completion, the mixture was concentrated in vacuo, and the crude product was diluted with water (50 mL) followed by extraction several times with EtOAc. The combined organic layer was dried over anhydrous sodium sulfate and concentrated in vacuo. The resultant product was purified by silica gel column chromatography to afford 550 mg of the desired product, methyl 2-(methyl(pyridin-3-ylmethyl)amino)-5-oxo-5H-thieno-[3,2-b]-pyran-6-carboxylate (30) as yellow solid; Yield: 56.8%; ¹H NMR (400 MHz, DMSO-d6): δ 8.54-8.51 (m, 3H), 7.71 (d, J=4.8 Hz, 1H), 7.40-7.37 (m, 1H), 6.42 (c, 1H), 4.76 (s, 2H), 3.68 (s, 3H),

Step 10

In a 100 mL single necked flask, Intermediate (30) (460 mg, 1.39 mmol) was dissolved in dry acetonitrile (60 mL). To the solution was added trimethylsilyl iodide (834 mg, 4.17 mmol) at rt, and the mixture was stirred for 30 min at rt. After the reaction completion, water (1 mL) was added into the mixture and a solid was precipitated. The resultant suspension was filtered, and the solid product obtained was purified by prep-HPLC to afford 110 mg of the desired product, 2-(methyl(pyridin-3-ylmethyl)amino)-5-oxo-5H-thieno-[3,2-b]pyran-6-carboxylic acid (Example 13) as a yellow solid; Yield: 25.5%; ¹H NMR (400 MHz, DMSO-d6): δ 8.70-8.67 (m, 2H), 8.59 (s, 1H), 8.03 (s, 1H), 7.69-7.67 (m, 1H), 6.52 (s, 1H), 4.86 (s, 2H), 3.22 (s, 3H); LC-MS (ESI): 317 (M+H)⁺; HPLC: 97.2%.

### Example 14

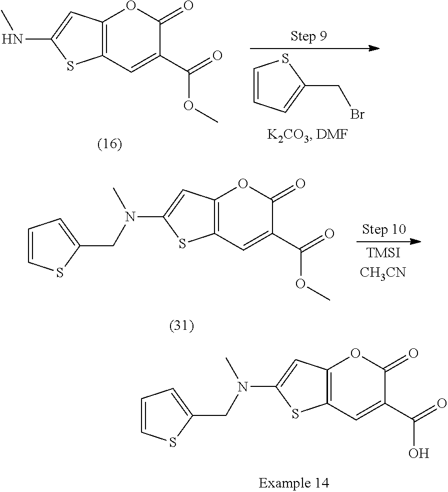

Step 9

To a solution of Intermediate (16) (500 mg, 2.09 mmol) from Example 4 and potassium carbonate (578 mg, 4.18 mmol) in DMF (20 mL) was added 2-(bromomethyl)thiophene (735 mg, 4.18 mmol) at rt, and the mixture was heated to 60° C. under N₂ atmosphere for 1 h. After the reaction completion, the mixture was concentrated in vacuo, and the product was treated with water (20 mL) and extracted with dichloromethane. The combined organic layer was dried over anhydrous sodium sulfate and concentrated in vacuo. The crude product obtained was purified by silica gel column chromatography to afford 300 mg of the desired product, methyl 2-(methyl (thiophen-2-ylmethyl)amino)-5-oxo-5H-thieno[3,2-b]pyran-6-carboxylate (31) as a brown solid; Yield, 42.8%; ¹H NMR (400 MHz, DMSO-d6): δ 8.55 (s, 1H), 7.48 (d, J=5.2 Hz, 1H), 7.15 (d, J=3.2 Hz, 1H), 7.01 (d, J=4.0 Hz, 1H), 6.44 (s, 1H), 4.88 (s, 2H), 3.68 (s, 3H), 3.11 (s, 3H).

Step 10

In a 100 mL single necked flask, Intermediate (31) (270 mg, 0.81 mmol) was dissolved in dry acetonitrile (50 mL). To the solution was added trimethylsilyl iodide (324 mg, 1.62 mmol) at rt, and the mixture was stirred for 2 h at rt. After reaction completion, water (1 mL) was added into the mixture and a solid was precipitated. The resultant suspension was filtered. The filter cake was washed with a small amount of methanol and dried in vacuo to afford 90 mg of the desired product, 2-(methyl(thiophen-2-ylmethyl)amino)-5-oxo-5H-thieno[3,2-b]pyran-6-carboxylic acid (Example 14) as yellow solid; Yield: 34.6%; ¹H NMR (400 MHz, DMSO-d6): δ 12.18 (s, 1H), 8.58 (s, 1H), 7.49 (d, J=8.2, 1H), 7.16 (s, 1H), 7.01 (s, 1H), 6.55 (s, 1H), 4.90 (s, 2H), 3.14 (s, 3H); LC-MS (ESI): 322 (M+H)⁺; HPLC: 96.3%.

### Example 15

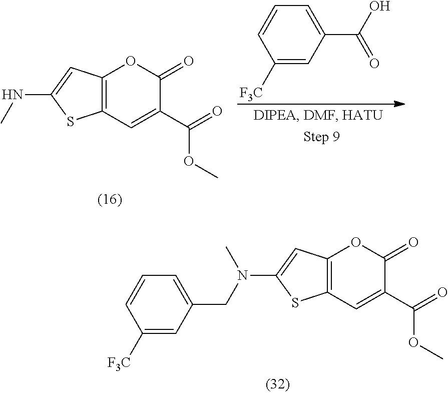

To a solution of Intermediate (16) (500 mg, 2.09 mmol, 1.0 eq) from Example 4 and HATU (1.19 g, 3.14 mmol, 1.5 eq) in DMF (50 mL), was added 3-(trifluoromethyl)benzoic acid (517 mg, 2.72 mmol, 1.3 eq) and DIEA (808 mg, 6.27 mmol, 3.0 eq) at rt. The mixture was stirred for 16 h at rt. The reaction was monitored by LCMS and HPLC. After the reaction completion, the solution was concentrated. The residue was extracted with DCM:MeOH=20:1 and water, washed with brine. The organic layer was dried with Na₂SO₄, concentrated for crude product. The crude was purified by FCC to afford 300 mg of the product, methyl 2-(N-methyl-3-(trifluoromethyl)benzamido)-5-oxo-5H-thieno[3,2-b]pyran-6-carboxylate (32); Yield 34.9%.

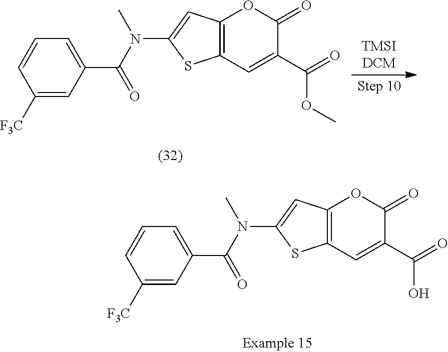 [SMILES: COC(=O)c1cc2sc(N(C)C(=O)c3cccc(C(F)(F)F)c3)cc2oc1=O ; CN(C(=O)c1cccc(C(F)(F)F)c1)c1cc2oc(=O)c(C(=O)O)cc2s1]

In a 250 mL single necked flask, intermediate (32) (210 mg, 0.63 mmol, 1.0 eq) was dissolver with DCM (100 mL). TMSI (376 mg, 1.88 mmol, 3.0 eq) was added to above solution at RT. The mixture was stirred for 18 h at RT. After the reaction completion, the mixture was concentrated to remove DCM, triturated with CH₃CN and water, filtered to afford 180 mg of the final product 2-((3-methoxybenzyl)(methyl)amino)-5-oxo-5H-thieno[3,2-b]pyran-6-carboxylic acid (Example 15); Yield: 88.7%; ¹H NMR (400 MHz, DMSO-d6) δ 12.68 (s, 1H), 8.88 (s, 1H), 8.04 (s, 1H), 7.94 (t, J=7.7 Hz, 2H), 7.76 (t, J=7.8 Hz, 1H), 7.16 (d, J=0.4 Hz, 1H), 3.45 (s, 3H), ¹⁹F NMR (332 MHz, DMSO-d6) δ −61.21 (s). LC-MS (ESI): 398.05 (M+H)⁺; HPLC: 220 nm, 95.5%; 254 nm, 95.6%.

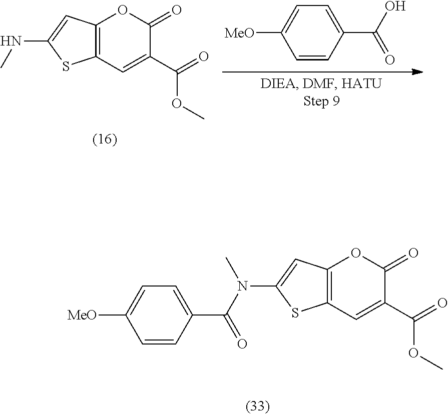

### Example 16

A solution of Intermediate (16) (500 mg, 2.09 mmol, 1.0 eq) and HATU (1.19 g, 3.14 mmol, 1.5 eq) in DMF (50 mL), was added 4-methoxybenzoic acid (413 mg, 2.72 mmol, 1.3 eq) and DIEA (808 mg, 6.27 mmol, 3.0 eq) at rt. The mixture was stirred for 16 h at rt. The reaction was monitored by LCMS and HPLC. After the reaction completion, the solution was concentrated. The residue was extracted with DCM:MeOH=20:1 and water, washed with brine. The organic layer was dried with Na₂SO₄, concentrated for crude product. The crude was purified by FCC to afford the 450 mg of methyl 2-(4-methoxy-N-methylbenzamido)-5-oxo-5H-thieno[3,2-b]pyran-6-carboxylate (33); Yield 57.6%.

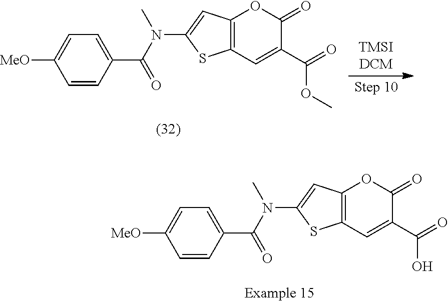 [SMILES: COC(=O)c1cc2sc(N(C)C(=O)c3ccc(OC)cc3)cc2oc1=O ; COc1ccc(C(=O)N(C)c2cc3oc(=O)c(C(=O)O)cc3s2)cc1]

In a 250 mL single necked flask, intermediate (33) (450 mg, 1.21 mmol, 1.0 eq) was dissolver with DCM (150 mL). TMSI (723 mg, 3.62 mmol, 3.0 eq) was added to above solution at RT. The mixture was stirred for 16 h at RT. After the reaction completion, the mixture was concentrated to remove DCM, triturated with CH₃CN and water, filtered to afford 320 mg of the final product, 2-(4-methoxy-N-methylbenzamido)-5-oxo-5H-thieno[3,2-b]pyran-6-carboxylic acid (Example 16); Yield: 73.9%; ¹H NMR (400 MHz, DMSO-d6) δ 12.61 (s, 1H), 8.85 (s, 1H), 7.62 (d, J=8.8 Hz, 1H), 7.09 (s, 1H), 7.76 (d, J=8.8 Hz, 1H), 3.81 (s, 3H), 3.52 (s, 3H). LC-MS (ESI): 360.00 (M+H)⁺; HPLC: 220 nm, 99.1%; 254 nm, 98.2%.

### Example 17

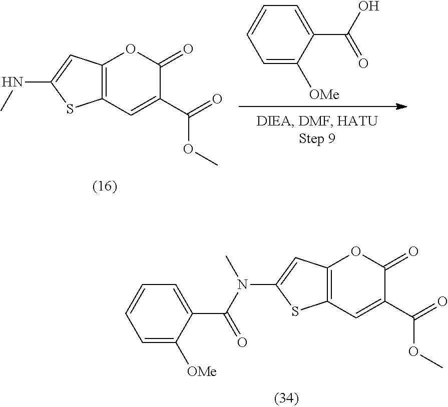

A solution of Intermediate (16) (500 mg, 2.09 mmol, 1.0 eq) and HATU (1.19 g, 3.14 mmol, 1.5 eq) in DMF (50 mL), was added 2-methoxybenzoic acid (413 mg, 2.72 mmol, 1.3 eq) and DIEA (808 mg, 6.27 mmol, 3.0 eq) at rt. The mixture was stirred for 16 h at rt. The reaction was monitored by LCMS and HPLC. After the reaction completion, the solution was concentrated. The residue was extracted with DCM:MeOH=20:1 and water, washed with brine. The organic layer was dried with Na₂SO₄, concentrated for crude product. The crude was purified by FCC to afford 477 mg of methyl 2-(2-methoxy-N-methylbenzamido)-5-oxo-5H-thieno[3,2-b]pyran-6-carboxylate (34); Yield 61.1%.

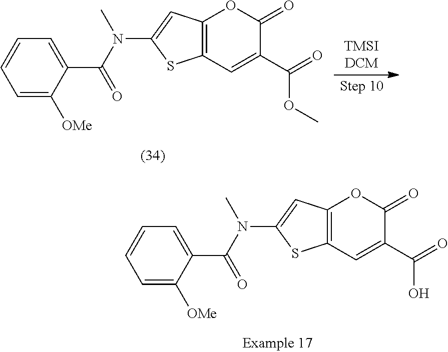 [SMILES: COC(=O)c1cc2sc(N(C)C(=O)c3ccccc3OC)cc2oc1=O ; COc1ccccc1C(=O)N(C)c1cc2oc(=O)c(C(=O)O)cc2s1]

In a 250 mL single necked flask, intermediate (34) (477 mg, 1.28 mmol, 1.0 eq) was dissolver with DCM (180 mL). TMSI (767 mg, 3.83 mmol, 3.0 eq) was added to above solution at RT. The mixture was stirred for 16 h at RT. After the reaction completion, the mixture was concentrated to remove DCM, triturated with CH₃CN and water, filtered to afford 376 mg of the final product, 2-(2-methoxy-N-methylbenzamido)-5-oxo-5H-thieno[3,2-b]pyran-6-carboxylic acid (Example 17); Yield: 81.9%; ¹H NMR (400 MHz, DMSO-d6) δ 12.67 (s, 1H), 8.86 (s, 1H), 7.52 (m, 1H), 7.40 (dd, 1H), 7.09-7.05 (m, 2H), 3.81 (s, 3H), 3.33 (s, 3H). LC-MS (ESI): 360.05 (M+H)⁺; HPLC: 220 nm, 99.4%; 254 nm, 99.0%.

### Example 18

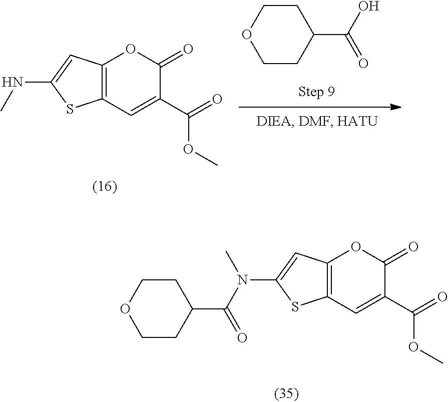

A solution of Intermediate (16) (500 mg, 2.09 mmol, 1.0 eq) and HATU (1.19 g, 3.14 mmol, 1.5 eq) in DMF (50 mL), was added tetrahydro-2H-pyran-4-carboxylic acid (353 mg, 2.72 mmol, 1.3 eq) and DIEA (808 mg, 6.27 mmol, 3.0 eq) at rt. The mixture was stirred for 16 h at rt. The reaction was monitored by LCMS and HPLC. After the reaction completion, Solution was concentrated. The residue was extracted with DCM:MeOH=20:1 and water, washed with brine. The organic layer was dried with anhydrous Na₂SO₄, concentrated for crude product. The crude was purified by FCC to afford 600 mg of methyl 2-(N-methyltetrahydro-2H-pyran-4-carboxamido)-5-oxo-5H-thieno[3,2-b]pyran-6-carboxylate (35); Yield 81.7%.

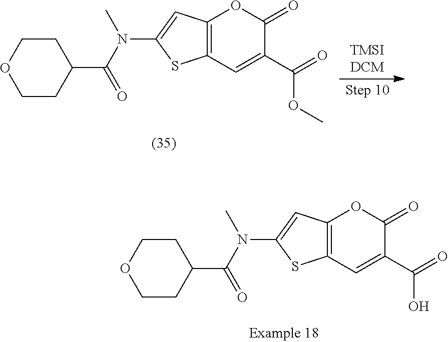 [SMILES: COC(=O)c1cc2sc(N(C)C(=O)C3CCOCC3)cc2oc1=O ; CN(C(=O)C1CCOCC1)c1cc2oc(=O)c(C(=O)O)cc2s1]

In a 250 mL single necked flask, intermediate (35) (300 mg, 0.85 mmol, 1.0 eq) was dissolver with DCM (120 mL). TMSI (512 mg, 2.56 mmol, 3.0 eq) was added to above solution at RT. The mixture was stirred for 16 h at RT. After the reaction completion, the mixture was concentrated, triturated with CH₃CN and water, filtered to afford 227 mg of the final product, 2-(N-methyltetrahydro-2H-pyran-4-carboxamido)-5-oxo-5H-thieno[3,2-b]pyran-6-carboxylic acid (Example 18); Yield: 78.8%; ¹H NMR (400 MHz, DMSO-d6) δ 12.59 (s, 1H), 8.80 (s, 1H), 7.03 (s, 1H), 3.86 (m, 2H), 3.58 (s, 3H), 3.42 (m, 3H), 1.71 (m, 2H), 1.62 (m, 2H). LC-MS (ESI): 338.05 (M+H)⁺; HPLC: 220 nm, 100%; 254 nm, 100%.

### Example 19

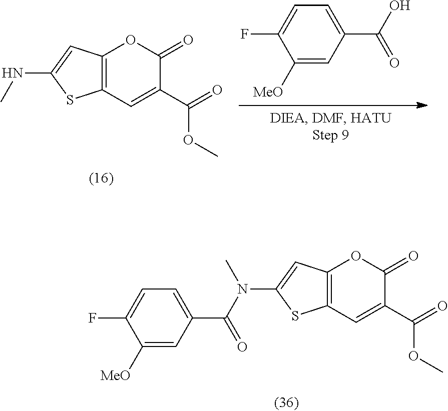

A solution of Intermediate (16) (500 mg, 2.09 mmol, 1.0 eq) and HATU (1.19 g, 3.14 mmol, 1.5 eq) in DMF (50 mL), was added 4-fluoro-3-methoxybenzoic acid (462 mg, 2.72 mmol, 1.3 eq) and DIEA (808 mg, 6.27 mmol, 3.0 eq) at rt. The mixture was stirred for 16 h at rt. The reaction was monitored by LCMS and HPLC. After the reaction completion, Solution was concentrated. The residue was extracted with DCM:MeOH=20:1 and water, washed with brine. The organic layer was dried with Na₂SO₄, concentrated for crude product. The crude was purified by FCC to afford 565 mg of methyl 2-(4-fluoro-3-methoxy-N-methylbenzamido)-5-oxo-5H-thieno[3,2-b]pyran-6-carboxylate (36); Yield 69.0%.

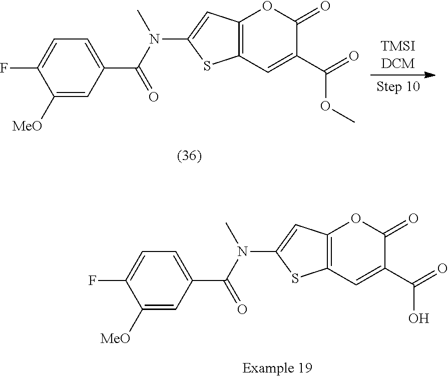 [SMILES: COC(=O)c1cc2sc(N(C)C(=O)c3ccc(F)c(OC)c3)cc2oc1=O ; COc1cc(C(=O)N(C)c2cc3oc(=O)c(C(=O)O)cc3s2)ccc1F]

In a 250 mL single necked flask, intermediate (36) (300 mg, 0.77 mmol, 1.0 eq) was dissolver with DCM (120 mL). TMSI (460 mg, 2.30 mmol, 3.0 eq) was added to above solution at RT. The mixture was stirred for 16 h at RT. After the reaction completion, the mixture was concentrated, triturated with CH₃CN and water, filtered to afford 250 mg of the final product, 2-(4-fluoro-3-methoxy-N-methylbenzamido)-5-oxo-5H-thieno[3,2-b]pyran-6-carboxylic acid (Example 19); Yield: 86.4%; ¹H NMR (400 MHz, DMSO-d6) δ 12.66 (s, 1H), 8.86 (s, 1H), 7.47 (dd, 1H), 7.36 (m, 1H), 7.22 (m, 1H), 7.12 (s, 1H), 3.86 (s, 3H), 3.58 (s, 3H), 3.48 (s, 3H); ¹⁹F NMR (332 MHz, DMSO-d6) δ −131.20 (m). LC-MS (ESI): 378.00 (M+H)⁺; HPLC: 220 nm, 97.6%; 254 nm, 97.0%.

### Example 20

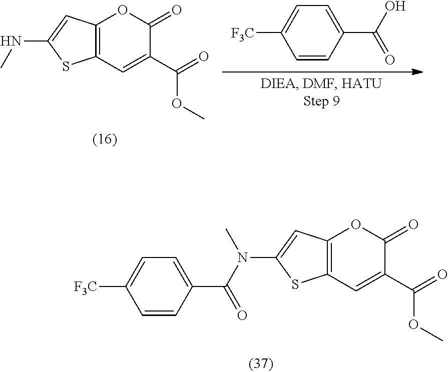

A solution of Intermediate (16) (500 mg, 2.09 mmol, 1.0 eq) and HATU (1.19 g, 3.14 mmol, 1.5 eq) in DMF (50 mL), was added 4-(trifluoromethyl)benzoic acid (517 mg, 2.72 mmol, 1.3 eq) and DIEA (808 mg, 6.27 mmol, 3.0 eq) at rt. The mixture was stirred for 16 h at rt. The reaction was monitored by LCMS and HPLC. After the reaction completion, Solution was concentrated. The residue was extracted with DCM:MeOH=20:1 and water, washed with brine. The organic layer was dried with Na₂SO₄, concentrated for crude product. The crude was purified by FCC to afford 247 mg of methyl 2-(N-methyl-4-(trifluoromethyl)benzamido)-5-oxo-5H-thieno[3,2-b]pyran-6-carboxylate (37); Yield 28.7%.

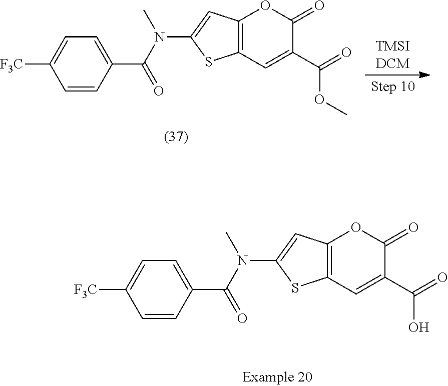 [SMILES: COC(=O)c1cc2sc(N(C)C(=O)c3ccc(C(F)(F)F)cc3)cc2oc1=O ; CN(C(=O)c1ccc(C(F)(F)F)cc1)c1cc2oc(=O)c(C(=O)O)cc2s1]

In a 250 mL single necked flask, intermediate (37) (207 mg, 0.50 mmol, 1.0 eq) was dissolver with DCM (60 mL). TMSI (604 mg, 3.02 mmol, 6.0 eq) was added to above solution at RT. The mixture was stirred for 5 d at RT. After the reaction completion, the mixture was concentrated, triturated with CH₃CN and water, filtered to afford 129 mg of the final product 2-(4-fluoro-3-methoxy-N-methylbenzamido)-5-oxo-5H-thieno[3,2-b]pyran-6-carboxylic acid (Example 20); Yield: 64.5%; ¹H NMR (400 MHz, DMSO-d6) δ 12.68 (s, 1H), 8.87 (s, 1H), 7.88 (q, 4H), 7.16 (s, 1H), 3.44 (s, 3H), ¹⁹F NMR (332 MHz, dmso) δ −61.46 (s), LC-MS (ESI): 461.05 (M+Na+MeCN)⁺; HPLC: 220 nm, 99.0%; 254 nm, 99.0%.

### Example 21

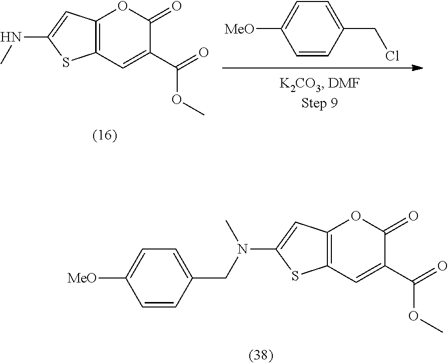

To a solution of intermediate (16) (500 mg, 2.09 mmol, 1.0 eq) and K₂CO₃ (865 mg, 6.27 mmol, 3.0 eq) in DMF (40 mL) was added 1-(chloromethyl)-4-methoxybenzene (491 mg, 3.14 mmol, 1.5 eq) at 70° C. The mixture was stirred for 3 h under N₂ at 70° C., monitored by TLC. After concentration, the residue was dissolved with DCM, filtered by silica gel, purified by FCC to afford 480 mg of methyl 2-((4-methoxybenzyl)(methyl)amino)-5-oxo-5H-thieno[3,2-b]pyran-6-carboxylate (38); Yield: 63.9%.

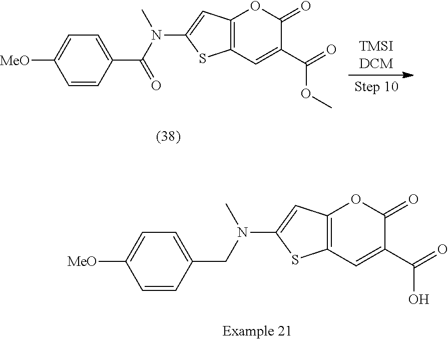 [SMILES: COC(=O)c1cc2sc(N(C)C(=O)c3ccc(OC)cc3)cc2oc1=O ; COc1ccc(CN(C)c2cc3oc(=O)c(C(=O)O)cc3s2)cc1]

In a 100 mL single necked flask, intermediate (38) (190 mg, 0.53 mmol, 1.0 eq) was dissolver with DCM (10 mL). TMSI (317 mg, 1.59 mmol, 3.0 eq) was added to above solution at 0° C. The mixture was stirred for 1 h at 0° C., monitored by TLC. The mixture was concentrated, triturated with CH₃CN and water, filtered and washed with MeOH to afford 105 mg of the final product, 2-((4-methoxybenzyl)(methyl)amino)-5-oxo-5H-thieno[3,2-b]pyran-6-carboxylic acid (Example 21); Yield: 57.5%; ¹H NMR (400 MEL, DMSO-d6): δ 12.15 (s, 1H), 8.53 (s, 1H), 7.22 (d, J=8.4 Hz, 2H), 6.92 (d, J=8.8 Hz, 2H), 6.51 (s, 1H), 4.64 (s, 2H), 3.71 (s, 3H), 3.13 (s, 3H); LCMS (ESI): 368.25 [M+Na]⁺, 713.45 [2M+Na]⁺; HPLC: 220 nm, 97.3%; 254 nm, 97.5%.

### Example 22

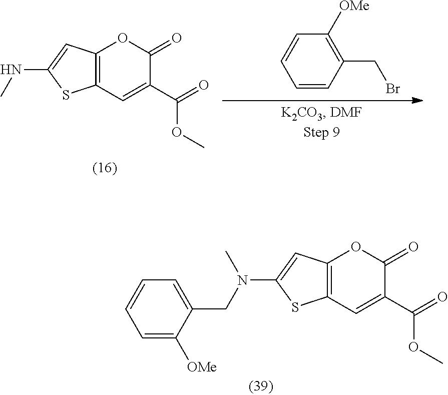

To a solution of intermediate (16) (500 mg, 2.09 mmol, 1.0 eq) and K₂CO₃ (865 mg, 6.27 mmol, 3.0 eq) in DMF (40 mL) was added 1-(bromomethyl)-2-methoxybenzene (631 mg, 3.14 mmol, 1.5 eq) at 70° C. The mixture was stirred for 1 h under N₂ at 70° C., monitored by TLC and LCMS. After concentration, the residue was dissolved with DCM, filtered by silica gel, purified by FCC to afford 480 mg of methyl 2-((2-methoxybenzyl)(methyl)amino)-5-oxo-5H-thieno[3,2-b]pyran-6-carboxylate (39); Yield: 46.6%.

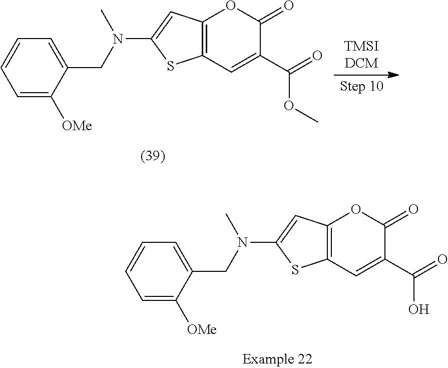 [SMILES: COC(=O)c1cc2sc(N(C)Cc3ccccc3OC)cc2oc1=O ; COc1ccccc1CN(C)c1cc2oc(=O)c(C(=O)O)cc2s1]

In a 50 mL single necked flask, intermediate (39) (270 mg, 0.75 mmol, 1.0 eq) was dissolver with DCM (10 mL). TMSI (451 mg, 2.25 mmol, 3.0 eq) was added to above solution at RT. The mixture was stirred for 1 h at RT, monitored by TLC and LCMS. The mixture was concentrated, triturated with CH₃CN and water, filtered, washed with MeOH to afford 170 mg of the final product, 2-((4-methoxybenzyl)(methyl)amino)-5-oxo-5H-thieno[3,2-b]pyran-6-carboxylic acid (Example 22); Yield: 65.5%; ¹H NMR (400 MHz, DMSO-d6): δ 12.16 (s, 1H), 8.51 (s, 1H), 7.30 (m, 1H), 7.11 (dd, 1H), 7.03 (dd, 1H), 6.91 (m, 1H), 4.64 (s, 2H), 3.78 (s, 3H), 3.14 (s, 3H); LCMS (ESI): 713.45 [2M+Na]⁺; HPLC: 220 nm, 98.2%; 254 nm, 98.0%.

### Example 23

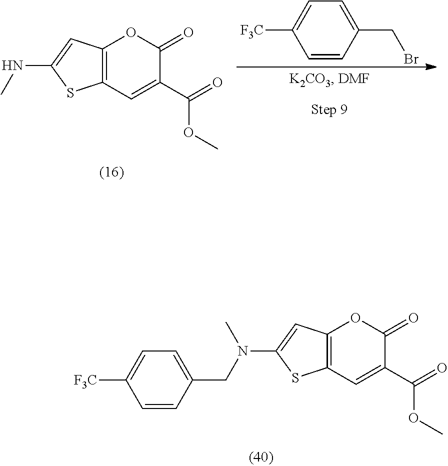

To a solution of intermediate (16) (500 mg, 2.09 mmol, 1.0 eq) and K₂CO₃ (865 mg, 6.27 mmol, 3.0 eq) in DMF (40 mL) was added 1-(bromomethyl)-4-(trifluoromethyl)benzene (749 mg, 3.13 mmol, 1.5 eq) at 70° C. The mixture was stirred for 1 h under N₂ at 70° C., monitored by TLC and LCMS. After concentration, the residue was dissolved with DCM, filtered by silica gel, purified by FCC to afford 370 mg of methyl 2-(methyl(4-(trifluoromethyl)benzyl)amino)-5-oxo-5H-thieno[3,2-b]pyran-6-carboxylate (40); Yield: 45.5%.

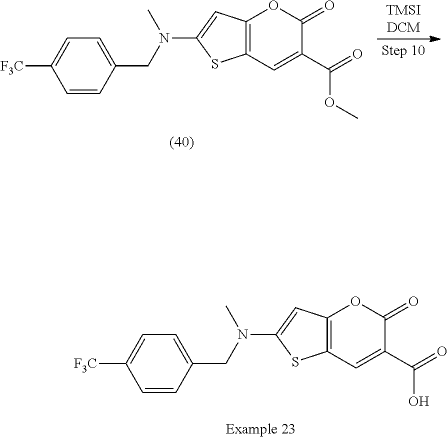 [SMILES: COC(=O)c1cc2sc(N(C)Cc3ccc(C(F)(F)F)cc3)cc2oc1=O ; CN(Cc1ccc(C(F)(F)F)cc1)c1cc2oc(=O)c(C(=O)O)cc2s1]

In a 50 mL single necked flask, intermediate (40) (270 mg, 0.68 mmol, 1.0 eq) was dissolver with DCM (10 mL). TMSI (408 mg, 2.04 mmol, 3.0 eq) was added to above solution at RT. The mixture was stirred for 16 h at RT, monitored by TLC and LCMS. The mixture was concentrated, triturated with CH₃CN and water, filtered, washed with MeOH to afford 195 mg of the final product, 2-(methyl(4-(trifluoromethyl)benzyl)amino)-5-oxo-5H-thieno[3,2-b]pyran-6-carboxylic acid (Example 23) 195 mg, Yield: 74.8%; ¹H NMR (400 MHz, DMSO-d6): δ 12.17 (s, 1H), 8.56 (s, 1H), 7.72 (d, J=8 Mz, 1H), 7.48 (d, J=8 Hz, 1H), 6.48 (s, 1H), 4.84 (s, 3H), 3.20 (s, 3H); LCMS (ESI): 384.05 [M+H]⁺; HPLC: 220 nm, 97.7%; 254 nm, 98.3%.

### Example 24

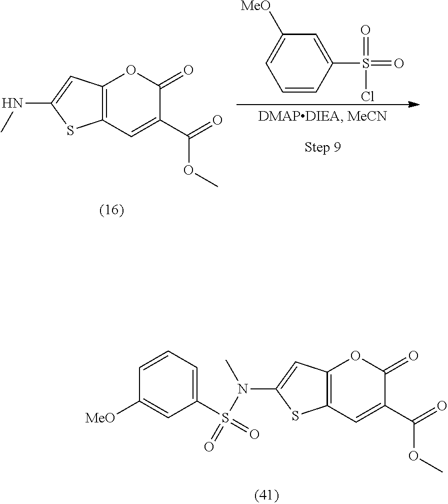

To a solution of intermediate (16) (500 mg, 2.09 mmol, 1.0 eq) and DMAP (128 mg, 1.05 mmol, 0.5 eq) in MeCN (50 mL) was added 3-methoxybenzenesulfonyl chloride (907 mg, 4.39 mmol, 2.1 eq) and DIEA (1.88 g, 14.63 mmol, 7.0 eq) at RT. The mixture was stirred for 16 h at RT, monitored by LCMS and HPLC. After concentration, the residue was triturated with MeOH and filtered to afford 719 mg of N-(6-((11-oxidanyl)carbonyl)-5-oxo-5H-thieno[3,2-b]pyran-2-yl)-3-methoxy-N-methylbenzenesulfonamide (41); yield 87.2%.

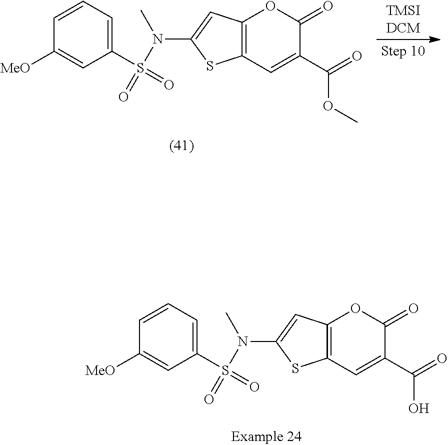 [SMILES: COC(=O)c1cc2sc(N(C)S(=O)(=O)c3cccc(OC)c3)cc2oc1=O ; COc1cccc(S(=O)(=O)N(C)c2cc3oc(=O)c(C(=O)O)cc3s2)c1]

In a 50 mL single necked flask, intermediate (41) (300 mg, 0.73 mmol, 1.0 eq) was dissolver with DCM (12 mL). TMSI (733 mg, 3.66 mmol, 5.0 eq) was added to above solution at RT. The mixture was stirred for 16 h at RT, monitored by LCMS and HPLC. The mixture was concentrated, triturated with CH₃CN and water, filtered, washed with MeOH and ether to afford 160 mg of the final product 2-((3-methoxy-N-methylphenyl)69ulfonamide)-5-oxo-5H-thieno[3,2-b]pyran-6-carboxylic acid (Example 24); Yield: 55.2%; ¹H NMR (400 MHz, DMSO-d6): δ 12.71 (s, 1H), 8.81 (s, 1H), 7.54 (t, J=8 Hz, 1H), 7.33 (m, 2H), 7.23 (t, J=2.2 Hz, 1H), 3.77 (s, 3H), 3.32 (s, 3H); LCMS (ESI): 418.10 [M+Na]⁺; HPLC: 220 nm, 99.6%; 254 nm, 99.4%.

### Example 25

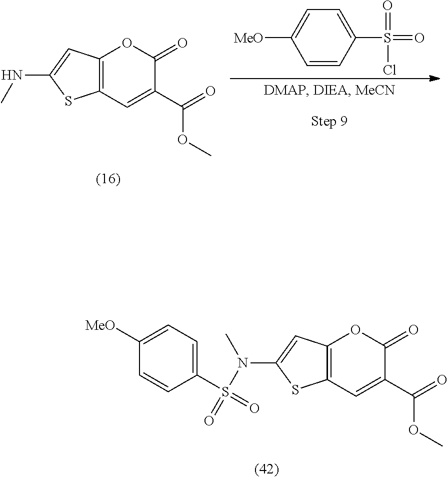

To a solution of intermediate (16) (400 mg, 1.67 mmol, 1.0 eq) and DMAP (102 mg, 0.84 mmol, 0.5 eq) in MeCN (40 mL) was added 4-methoxybenzenesulfonyl chloride (518 mg, 2.51 mmol, 1.5 eq) and DIEA (1.51 g, 11.69 mmol, 7.0 eq) at RT. The mixture was stirred for 16 h at RT, monitored by LCMS and HPLC. After concentration, the residue was triturated with MeOH and filtered to afford 450 mg of methyl 2-((4-methoxy-N-methylphenyl)70ulfonamide)-5-oxo-5H-thieno[3,2-b]pyran-6-carboxylate (42); yield 65.7%.

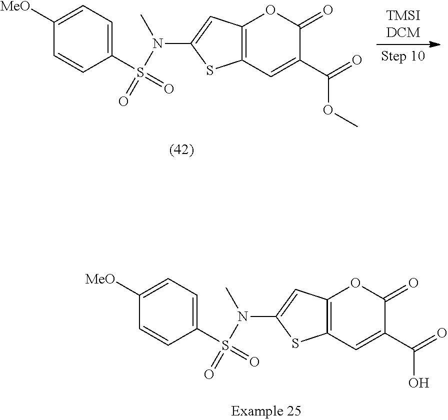 [SMILES: COC(=O)c1cc2sc(N(C)S(=O)(=O)c3ccc(OC)cc3)cc2oc1=O ; COc1ccc(S(=O)(=O)N(C)c2cc3oc(=O)c(C(=O)O)cc3s2)cc1]

In a 100 mL single necked flask, intermediate (42) (432 mg, 1.06 mmol, 1.0 eq) was dissolver with DCM (20 mL). TMSI (987 mg, 4.93 mmol, 4.6 eq) was added to above solution at RT. The mixture was stirred for 48 h at RT, monitored by LCMS and HPLC. The mixture was concentrated, triturated with CH₃CN and water, filtered, washed with MeOH and ether to afford 270 mg of the final product 2-((4-methoxy-N-methylphenyl)sulfonamido)-5-oxo-5H-thieno[3,2-b]pyran-6-carboxylic acid (Example 25); Yield: 64.7%; ¹H NMR (400 MHz, DMSO-d6): δ 12.75 (s, 1H), 8.81 (s, 1H), 7.73 (d, J=8.8 Hz, 2H), 7.12 (d, J=8.8 Hz, 2H), 6.88 (s, 1H), 3.80 (s, 3H), 3.28 (s, 3H); LCMS (ESI): 396.00 [M+H]⁺; HPLC: 220 nm, 100%; 254 nm, 100%.

### Example 26

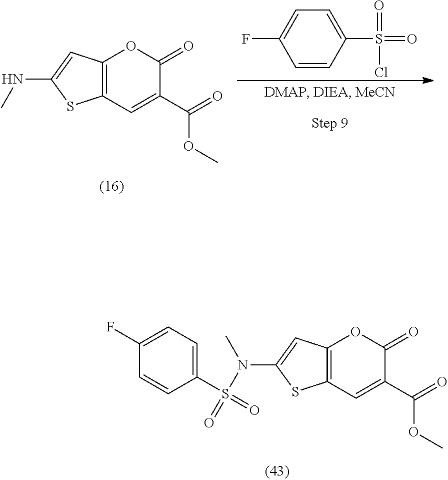

To a solution of intermediate (16) (310 mg, 1.3 mmol, 1.0 eq) and DMAP (83 mg, 0.68 mmol, 0.5 eq) in MeCN (30 mL) was added 4-fluorobenzenesulfonyl chloride (380 mg, 1.94 mmol, 1.5 eq) and DIEA (1.26 g, 9.77 mmol, 7.5 eq) at RT. The mixture was stirred for 16 h at RT, monitored by LCMS and HPLC. After concentration, the residue was triturated with MeOH, filtered to afford 434 mg of methyl 2-((4-fluoro-N-methylphenyl)sulfonamido)-5-oxo-5H-thieno[3,2-b]pyran-6-carboxylate (43); yield 84.2%.

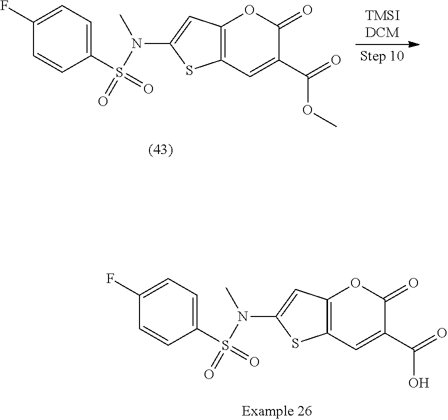 [SMILES: COC(=O)c1cc2sc(N(C)S(=O)(=O)c3ccc(F)cc3)cc2oc1=O ; CN(c1cc2oc(=O)c(C(=O)O)cc2s1)S(=O)(=O)c1ccc(F)cc1]

In a 100 mL single necked flask, intermediate (43) (434 mg, 1.09 mmol, 1.0 eq) was dissolver with DCM (40 mL). TMSI (633 mg, 3.28 mmol, 3 eq) was added to above solution at RT. The mixture was stirred for 16 h at RT, monitored by LCMS and HPLC. The mixture was concentrated, triturated with CH₃CN and water, filtered, washed with MeOH and ether to afford 370 mg of the final product, 2-((4-fluoro-N-methylphenyl)sulfonamido)-5-oxo-5H-thieno[3,2-b]pyran-6-carboxylic acid (Example 26); Yield: 88.3%; ¹H NMR (400 MHz, CDCl₃): δ 12.10 (s, 1H), 8.76 (s, 1H), 7.82 (m, 2H), 7.21 (d, J=8 Hz, 2H), 6.69 (s, 1H), 3.38 (s, 3H); ¹⁹F NMR (332 MHz, CDCl₃): δ −100.90 (s); LCMS (ESI): 406.65 [M+Na]⁺; HPLC: 220 nm, 97.9%; 254 nm, 96.5%.

### Example 27

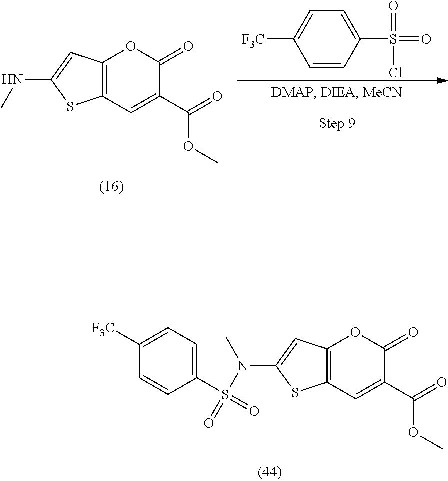

To a solution of intermediate (16) (400 mg, 1.67 mmol, 1.0 eq) and DMAP (102 mg, 0.83 mmol, 0.5 eq) in MeCN (40 mL) was added 4-(trifluoromethyl)benzenesulfonyl chloride (914 mg, 3.73 mmol, 2.2 eq) and DIEA (1.51 g, 11.68 mmol, 7.0 eq) at RT. The mixture was stirred for 48 h at RT, monitored by LCMS and HPLC. After concentration, the residue was triturated with MeOH, filtered to afford 544 mg of methyl 2-4N-methyl-4-(trifluoromethyl)phenyl)sulfonamido)-5-oxo-5H-thieno[3,2-b]pyran-6-carboxylate (44); yield 72.7%.

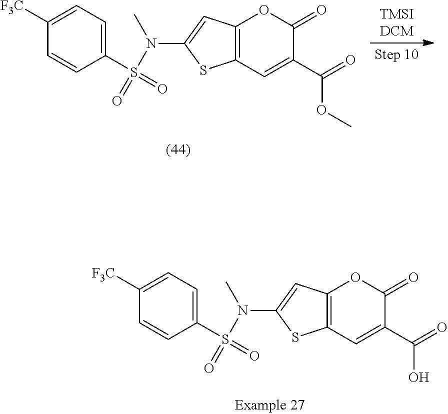 [SMILES: COC(=O)c1cc2sc(N(C)S(=O)(=O)c3ccc(C(F)(F)F)cc3)cc2oc1=O ; CN(c1cc2oc(=O)c(C(=O)O)cc2s1)S(=O)(=O)c1ccc(C(F)(F)F)cc1]

In a 250 mL single necked flask, intermediate (44) (544 mg, 1.22 mmol, 1.0 eq) was dissolver with DCM (100 mL). TMSI (730 mg, 3.65 mmol, 3 eq) was added to above solution at RT. The mixture was stirred for 48 h at RT, monitored by LCMS and HPLC. The mixture was concentrated, triturated with CH₃CN and water, filtered, washed with MeOH and ether to afford 400 mg of the final product 2-((N-methyl-4-(trifluoromethyl)phenyl)sulfonamido)-5-oxo-5H-thieno[3,2-b]pyran-6-carboxylic acid (Example 27); Yield: 75.9%; ¹H NMR (400 MHz, DMSO-d6): δ 12.83 (s, 1H), 8.83 (s, 1H), 8.01 (q, 4H), 6.98 (s, 1H), 3.34 (s, 3H); ¹⁹F NMR (332 MHz, DMSO): δ 61.86 (s); LCMS (ESI): 456.05 [M+Na]⁺; HPLC: 220 nm, 100%; 254 nm, 100%.

### Example 28

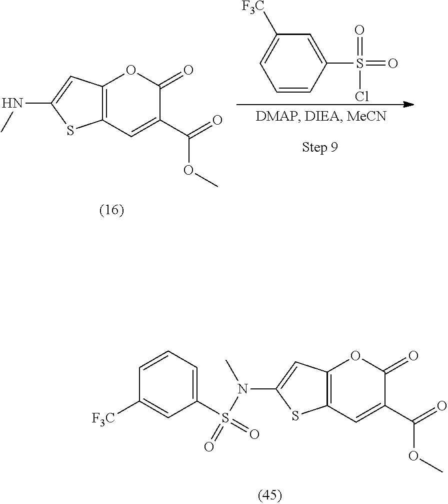

To a solution of intermediate (16) (400 mg, 1.67 mmol, 1.0 eq) and DMAP (102 mg, 0.83 mmol, 0.5 eq) in MeCN (40 mL) was added 3-(trifluoromethyl)benzenesulfonyl chloride (614 mg, 2.51 mmol, 1.5 eq) and DIEA (1.51 g, 11.68 mmol, 7.0 eq) at RT. The mixture was stirred for 48 h at RT, monitored by LCMS and HPLC. After concentration, the residue was triturated with MeOH, filtered to afford 459 mg of methyl 2-4N-methyl-3-(trifluoromethyl)phenyl)sulfonamido)-5-oxo-5H-thieno[3,2-b]pyran-6-carboxylate (45); Yield 61.3%.

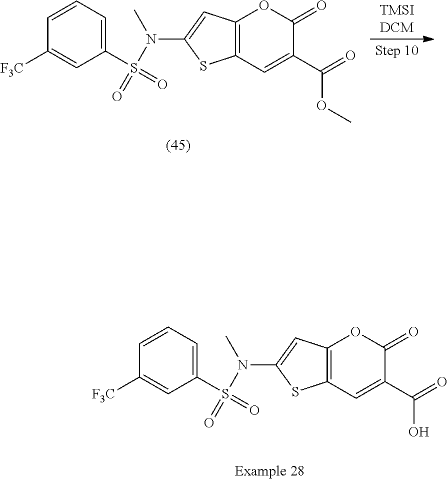 [SMILES: COC(=O)c1cc2sc(N(C)S(=O)(=O)c3cccc(C(F)(F)F)c3)cc2oc1=O ; CN(c1cc2oc(=O)c(C(=O)O)cc2s1)S(=O)(=O)c1cccc(C(F)(F)F)c1]

In a 250 mL single necked flask, intermediate (45)6-2 (459 mg, 1.03 mmol, 1.0 eq) was dissolver with DCM (100 mL). TMSI (1.03 g, 5.13 mmol, 5 eq) was added to above solution at RT. The mixture was stirred for 48 h at RT, monitored by LCMS and HPLC. The mixture was concentrated, triturated with CH₃CN and water, filtered, washed with MeOH and ether to afford 290 mg of the final product, 2-((N-methyl-3-(trifluoromethyl)phenyl)sulfonamido)-5-oxo-5H-thieno[3,2-b]pyran-6-carboxylic acid (Example 28); Yield: 65.2%; ¹H NMR (400 MHz, DMSO): δ 12.86 (s, 1H), 8.83 (s, 1H), 8.17 (d, J=4 Hz, 1H), 8.05 (t, J=8 Hz, 2H), 7.88 (t, J=8 Hz, 1H), 6.98 (s, 1H), 3.35 (s, 3H); ¹⁹F NMR (332 MHz, DMSO-d6): δ 61.43 (s); LCMS (ESI): 434.05 [M+H]⁺; HPLC: 220 nm, 100%; 254 nm, 100%.

TABLE 1

| | | | Cytotoxicity |
|---|---|---|---|
| Example | Structure | Name | [IC₅₀; uM] |
| 1 | 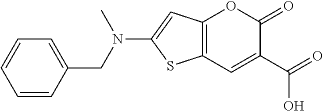 [SMILES: CN(Cc1ccccc1)c1cc2oc(=O)c(C(=O)O)cc2s1] | 2-(benzyl(methyl)amino)-5-oxo- 5H-thieno[3,2-b]pyran-6- carboxylic acid | A |
| 2 | 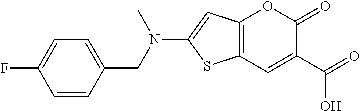 [SMILES: CN(Cc1ccc(F)cc1)c1cc2oc(=O)c(C(=O)O)cc2s1] | 2-((4-fluorobenzyl)(methyl) amino)-5-oxo-5H-thieno[3,2- b]pyran-6-carboxylic acid | A |
| 3 | 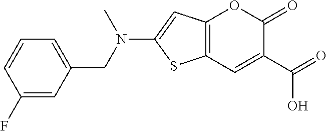 [SMILES: CN(Cc1cccc(F)c1)c1cc2oc(=O)c(C(=O)O)cc2s1] | 2-((3-fluorobenzyl)(methyl) amino)-5-oxo-5H-thieno[3,2- b]pyran-6-carboxylic acid | A |
| 4 | 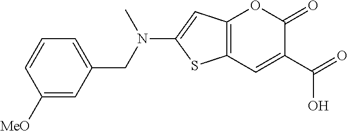 [SMILES: COc1cccc(CN(C)c2cc3oc(=O)c(C(=O)O)cc3s2)c1] | 2-((3-methoxybenzyl)(methyl) amino)-5-oxo-5H-thieno[3,2- b]pyran-6-carboxylic acid | A |
| 5 | 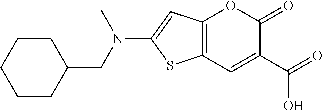 [SMILES: CN(CC1CCCCC1)c1cc2oc(=O)c(C(=O)O)cc2s1] | 2-((cyclohexylmethyl)(methyl) amino)-5-oxo-5H-thieno[3,2-b] pyran-6-carboxylic acid | A |
| 6 | 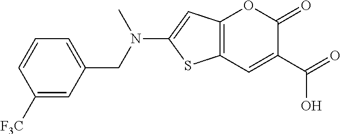 [SMILES: CN(Cc1cccc(C(F)(F)F)c1)c1cc2oc(=O)c(C(=O)O)cc2s1] | 2-(methyl(3-(trifluoromethyl) benzyl)amino)-5-oxo-5H- thieno[3,2-b]pyran-6-carboxylic acid | A |
| 7 | 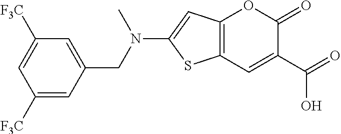 [SMILES: CN(Cc1cc(C(F)(F)F)cc(C(F)(F)F)c1)c1cc2oc(=O)c(C(=O)O)cc2s1] | 2-((3,5-bis(trifluoromethyl) benzyl)(methyl)amino)-5-oxo- 5H-thieno[3,2-b]pyran-6- carboxylic acid | A |
| 8 | 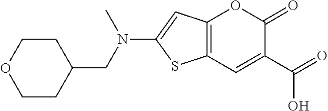 [SMILES: CN(CC1CCOCC1)c1cc2oc(=O)c(C(=O)O)cc2s1] | 2-(methyl((tetrahydro-2H- pyran-4-yl)methyl)amino)- 5-oxo-5H-thieno[3,2-b] pyran-6-carboxylic acid | A |
| 9 | 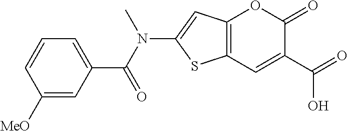 [SMILES: COc1cccc(C(=O)N(C)c2cc3oc(=O)c(C(=O)O)cc3s2)c1] | 2-(3-methoxy- N-methylbenzamido)- 5-oxo-5H-thieno[3,2-b] pyran-6-carboxylic acid | A |
| 10 | 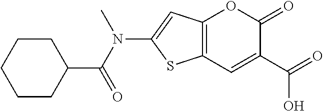 [SMILES: CN(C(=O)C1CCCCC1)c1cc2oc(=O)c(C(=O)O)cc2s1] | 2-(N- methylcyclohexanecarboxamido)- 5-oxo-5H-thieno[3,2-b]pyran- 6-carboxylic acid | A |
| 11 | 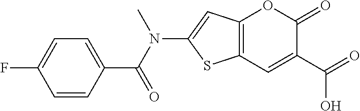 [SMILES: CN(C(=O)c1ccc(F)cc1)c1cc2oc(=O)c(C(=O)O)cc2s1] | 2-(4-fluoro-N- methylbenzamido)-5-oxo- 5H-thieno[3,2-b]pyran-6- carboxylic acid | A |
| 12 | 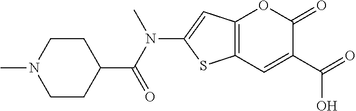 [SMILES: CN1CCC(C(=O)N(C)c2cc3oc(=O)c(C(=O)O)cc3s2)CC1] | 2-(methyl((1-methylpiperidin- 4-yl)methyl)amino)-5-oxo-5H- thieno[3,2-b]pyran-6-carboxylic acid | A |
| 13 | 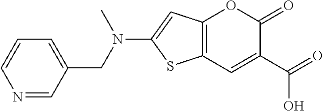 [SMILES: CN(Cc1cccnc1)c1cc2oc(=O)c(C(=O)O)cc2s1] | 2-(methyl(pyridin-3-ylmethyl) amino)-5-oxo-5H-thieno[3,2- b]pyran-6-carboxylic acid | A |
| 14 | 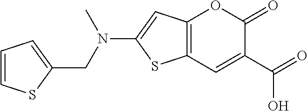 [SMILES: CN(Cc1cccs1)c1cc2oc(=O)c(C(=O)O)cc2s1] | 2-(methyl(thiophen-2-ylmethyl) amino)-5-oxo-5H-thieno[3,2-b] pyran-6-carboxylic acid | A |
| 15 | 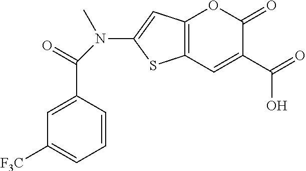 [SMILES: CN(C(=O)c1cccc(C(F)(F)F)c1)c1cc2oc(=O)c(C(=O)O)cc2s1] | 2-[Methyl-(3-trifluoromethyl- benzoyl)-amino]-5-oxo-5H- thieno[3,2-b]pyran-6-carboxylic acid | A |
| 16 | 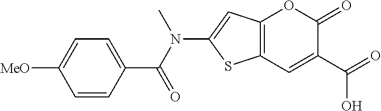 [SMILES: COc1ccc(C(=O)N(C)c2cc3oc(=O)c(C(=O)O)cc3s2)cc1] | 2-[(4-Methoxy-benzoyl)-methyl- amino]-5-oxo-5H-thieno[3,2-b] pyran-6-carboxylic acid | A |
| 17 | 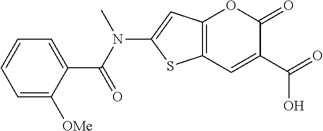 [SMILES: COc1ccccc1C(=O)N(C)c1cc2oc(=O)c(C(=O)O)cc2s1] | 2-[(2-Methoxy-benzoyl)-methyl- amino]-5-oxo-5H-thieno[3,2-b] pyran-6-carboxylic acid | A |
| 18 | 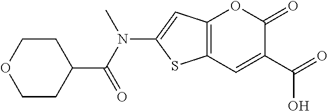 [SMILES: CN(C(=O)C1CCOCC1)c1cc2oc(=O)c(C(=O)O)cc2s1] | 2-[Methyl-(tetrahydro-pyran-4- carbonyl)-amino]-5-oxo-5H- thieno[3,2-b]pyran-6-carboxylic acid | A |
| 19 | 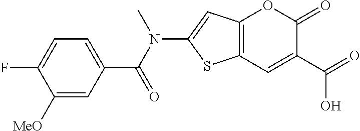 [SMILES: COc1cc(C(=O)N(C)c2cc3oc(=O)c(C(=O)O)cc3s2)ccc1F] | 2-[(4-Fluoro-3-methoxy-benzoyl)- methyl-amino]-5-oxo-5H-thieno [3,2-b]pyran-6-carboxylic acid | A |
| 20 | 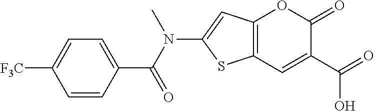 [SMILES: CN(C(=O)c1ccc(C(F)(F)F)cc1)c1cc2oc(=O)c(C(=O)O)cc2s1] | 2-[Methyl-(4-trifluoromethyl- benzoyl)-amino]-5-oxo-5H- thieno[3,2-b]pyran-6-carboxylic acid | A |
| 21 | 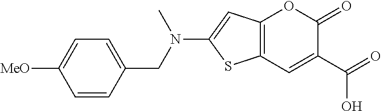 [SMILES: COc1ccc(CN(C)c2cc3oc(=O)c(C(=O)O)cc3s2)cc1] | 2-[(4-Methoxy-benzyl)-methyl- amino]-5-oxo-5H-thieno[3,2-b] pyran-6-carboxylic acid | A |
| 22 | 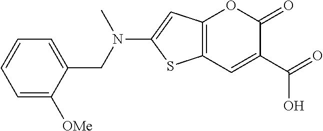 [SMILES: COc1ccccc1CN(C)c1cc2oc(=O)c(C(=O)O)cc2s1] | 2-[(2-Methoxy-benzyl)-methyl- amino]-5-oxo-5H-thieno[3,2-b] pyran-6-carboxylic acid | A |
| 23 | 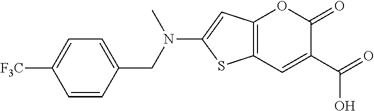 [SMILES: CN(Cc1ccc(C(F)(F)F)cc1)c1cc2oc(=O)c(C(=O)O)cc2s1] | 2-[Methyl-(4-trifluoromethyl- benzyl)-amino]-5-oxo-5H- thieno[3,2-b]pyran-6-carboxylic acid | A |
| 24 | 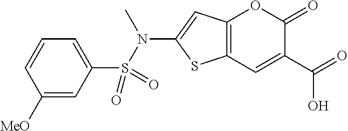 [SMILES: COc1cccc(S(=O)(=O)N(C)c2cc3oc(=O)c(C(=O)O)cc3s2)c1] | 2-[(3-Methoxy-benzenesulfonyl)- methyl-amino]-5-oxo-5H-thieno [3,2-b]pyran-6-carboxylic acid | A |
| 25 | 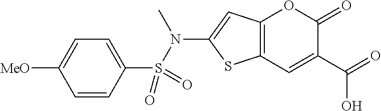 [SMILES: COc1ccc(S(=O)(=O)N(C)c2cc3oc(=O)c(C(=O)O)cc3s2)cc1] | 2-[(4-Methoxy-benzenesulfonyl)- methyl-amino]-5-oxo-5H-thieno [3,2-b]pyran-6-carboxylic acid | A |
| 26 | 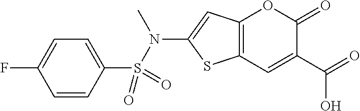 [SMILES: CN(c1cc2oc(=O)c(C(=O)O)cc2s1)S(=O)(=O)c1ccc(F)cc1] | 2-[(4-Fluoro-benzenesulfonyl)- methyl-amino]-5-oxo-5H-thieno [3,2-b]pyran-6-carboxylic acid | A |
| 27 | 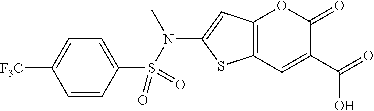 [SMILES: CN(c1cc2oc(=O)c(C(=O)O)cc2s1)S(=O)(=O)c1ccc(C(F)(F)F)cc1] | 2-[Methyl-(4-trifluoromethyl- benzenesulfonyl)-amino]-5-oxo- 5H-thieno[3,2-b]pyran-6- carboxylic acid | A |
| 28 | 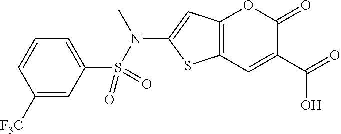 [SMILES: CN(c1cc2oc(=O)c(C(=O)O)cc2s1)S(=O)(=O)c1cccc(C(F)(F)F)c1] | 2-[Methyl-(3-trifluoromethyl- benzenesulfonyl)-amino]-5-oxo- 5H-thieno[3,2-b]pyran-6- carboxylic acid | A |
| 29 | 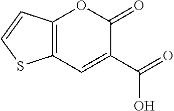 [SMILES: O=C(O)c1cc2sccc2oc1=O] | 5-oxo-5H-thieno[3,2-b]pyran- 6-carboxylic acid | B |
| 30 | 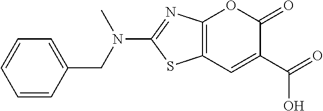 [SMILES: CN(Cc1ccccc1)c1nc2oc(=O)c(C(=O)O)cc2s1] | 2-(benzyl(methyl)amino)-5-oxo- 5H-pyrano[2,3-d]thiazole-6- carboxylic acid | NT |
| 31 | 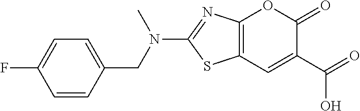 [SMILES: CN(Cc1ccc(F)cc1)c1nc2oc(=O)c(C(=O)O)cc2s1] | 2-((4-fluorobenzyl)(methyl)amino)- 5-oxo-5H-pyrano[2,3-d]thiazole- 6-carboxylic acid | NT |
| 32 | 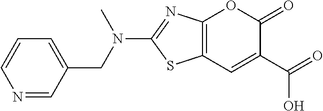 [SMILES: CN(Cc1cccnc1)c1nc2oc(=O)c(C(=O)O)cc2s1] | 2-(methyl(pyridin-3-ylmethyl) amino)-5-oxo-5H-pyrano[2,3- d]thiazole-6-carboxylic acid | NT |
| 33 | 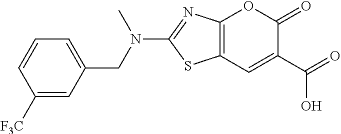 [SMILES: CN(Cc1cccc(C(F)(F)F)c1)c1nc2oc(=O)c(C(=O)O)cc2s1] | 2-(methyl(3-(trifluoromethyl) benzyl)amino)-5-oxo-5H- pyrano[2,3-d]thiazole-6- carboxylic acid | NT |
| 34 | 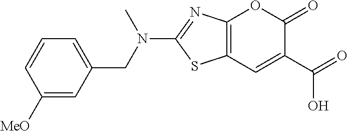 [SMILES: COc1cccc(CN(C)c2nc3oc(=O)c(C(=O)O)cc3s2)c1] | 2-((3-methoxybenzyl)(methyl) amino)-5-oxo-5H-pyrano[2,3-d] thiazole-6-carboxylic acid | NT |
| 35 | 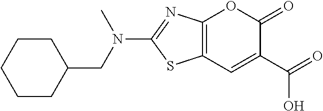 [SMILES: CN(CC1CCCCC1)c1nc2oc(=O)c(C(=O)O)cc2s1] | 2-((cyclohexylmethyl)(methyl) amino)-5-oxo-5H-pyrano[2,3-d] thiazole-6-carboxylic acid | NT |
| 36 | 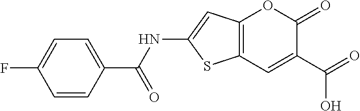 [SMILES: O=C(Nc1cc2oc(=O)c(C(=O)O)cc2s1)c1ccc(F)cc1] | 2-(4-fluorobenzamido)-5-oxo- 5H-thieno[3,2-b]pyran-6- carboxylic acid | NT |
| 37 | 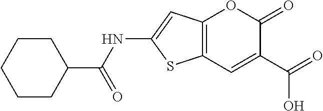 [SMILES: O=C(O)c1cc2sc(NC(=O)C3CCCCC3)cc2oc1=O] | 2-(cyclohexanecarboxamido)-5- oxo-5H-thieno[3,2-b]pyran- 6-carboxylic acid | NT |
| 38 | 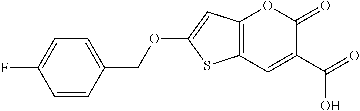 [SMILES: O=C(O)c1cc2sc(OCc3ccc(F)cc3)cc2oc1=O] | 2-((4-fluorobenzyl)oxy)-5-oxo- 5H-thieno[3,2-b]pyran-6- carboxylic acid | NT |
| 39 | 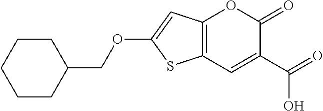 [SMILES: O=C(O)c1cc2sc(OCC3CCCCC3)cc2oc1=O] | 2-(cyclohexylmethoxy)-5-oxo- 5H-thieno[3,2-b]pyran-6- carboxylic acid | NT |
| 40 | 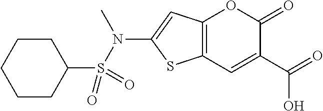 [SMILES: CN(c1cc2oc(=O)c(C(=O)O)cc2s1)S(=O)(=O)C1CCCCC1] | 2-(Cyclohexanesulfonyl-methyl- amino)-5-oxo-5H-thieno[3,2-b] pyran-6-carboxylic acid | NT |
| 41 | 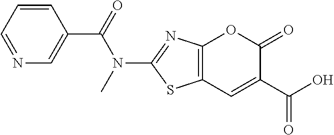 [SMILES: CN(C(=O)c1cccnc1)c1nc2oc(=O)c(C(=O)O)cc2s1] | 2-[Methyl-(pyridine-3-carbonyl)- amino]-5-oxo-5H-thieno[3,2-b] pyran-6-carboxylic acid | NT |
| 42 | 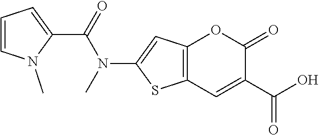 [SMILES: CN(C(=O)c1cccn1C)c1cc2oc(=O)c(C(=O)O)cc2s1] | 2-[Methyl-(1-methyl-1H-pyrrole- 2-carbonyl)-amino]-5-oxo-5H- thieno[3,2-b]pyran-6-carboxylic acid | NT |
| 43 | 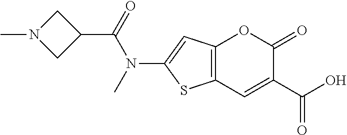 [SMILES: CN1CC(C(=O)N(C)c2cc3oc(=O)c(C(=O)O)cc3s2)C1] | 2-[Methyl-(1-methyl-azetidine- 3-carbonyl)-amino]-5-oxo-5H- thieno[3,2-b]pyran-6-carboxylic acid | NT |
| 44 | 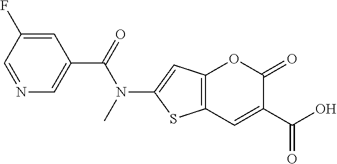 [SMILES: CN(C(=O)c1cncc(F)c1)c1cc2oc(=O)c(C(=O)O)cc2s1] | 2-[(5-Fluoro-pyridine-3-carbonyl)- methyl-amino]-5-oxo-5H-thieno [3,2-b]pyran-6-carboxylic acid | NT |
| 45 | 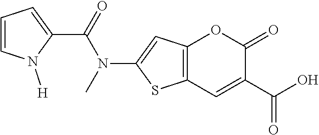 [SMILES: CN(C(=O)c1ccc[nH]1)c1cc2oc(=O)c(C(=O)O)cc2s1] | 2-[Methyl-(1H-pyrrole-2-carbonyl)- amino]-5-oxo-5H-thieno[3,2-b] pyran-6-carboxylic acid | NT |
| 46 | 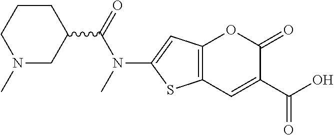 [SMILES: CN1CCCC(C(=O)N(C)c2cc3oc(=O)c(C(=O)O)cc3s2)C1] | 2-[Methyl-(1-methyl-piperidine-3- carbonyl)-amino]-5-oxo-5H-thieno [3,2-b]pyran-6-carboxylic acid | NT |
| 47 | 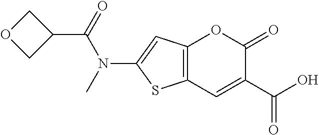 [SMILES: CN(C(=O)C1COC1)c1cc2oc(=O)c(C(=O)O)cc2s1] | 2-[Methyl-(oxetane-3-carbonyl)- amino]-5-oxo-5H-thieno[3,2-b] pyran-6-carboxylic acid | NT |
| 48 | 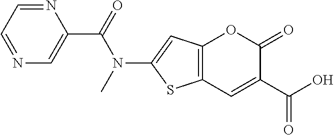 [SMILES: CN(C(=O)c1cnccn1)c1cc2oc(=O)c(C(=O)O)cc2s1] | 2-[Methyl-(pyrazine-2-carbonyl)- amino]-5-oxo-5H-thieno[3,2-b] pyran-6-carboxylic acid | NT |
| 49 | 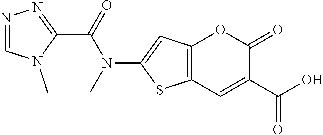 [SMILES: CN(C(=O)c1nncn1C)c1cc2oc(=O)c(C(=O)O)cc2s1] | 2-[Methyl-(4-methyl-4H-[1,2,4] triazole-3-carbonyl)-amino]-5-oxo- 5H-thieno[3,2-b]pyran-6-carboxylic acid | NT |
| 50 | 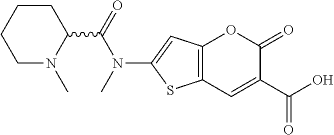 [SMILES: CN(C(=O)C1CCCCN1C)c1cc2oc(=O)c(C(=O)O)cc2s1] | 2-[Methyl-(1-methyl-piperidine- 2-carbonyl)-amino]-5-oxo-5H- thieno[3,2-b]pyran-6-carboxylic acid | NT |
| 51 | 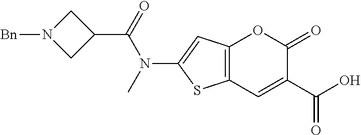 [SMILES: CN(C(=O)C1CN(Cc2ccccc2)C1)c1cc2oc(=O)c(C(=O)O)cc2s1] | 2-[(1-Benzyl-azetidine-3-carbonyl)- methyl-amino]-5-oxo-5H-thieno[3,2- b]pyran-6-carboxylic acid | NT |
| 52 | 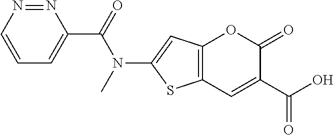 [SMILES: CN(C(=O)c1cccnn1)c1cc2oc(=O)c(C(=O)O)cc2s1] | 2-[Methyl-(pyridazine-3-carbonyl)- amino]-5-oxo-5H-thieno[3,2-b] pyran-6-carboxylic acid | NT |
| 53 | 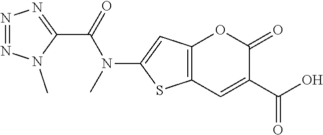 [SMILES: CN(C(=O)c1nnnn1C)c1cc2oc(=O)c(C(=O)O)cc2s1] | 2-[Methyl-(1-methyl-1H-tetrazole-5- carbonyl)-amino]-5-oxo-5H-thieno [3,2-b]pyran-6-carboxylic acid | NT |
| 54 | 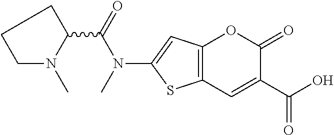 [SMILES: CN(C(=O)C1CCCN1C)c1cc2oc(=O)c(C(=O)O)cc2s1] | 2-[Methyl-(1-methyl-pyrrolidine-2- carbonyl)-amino]-5-oxo-5H-thieno [3,2-b]pyran-6-carboxylic acid | NT |
| 55 | 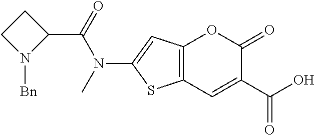 [SMILES: CN(C(=O)C1CCN1Cc1ccccc1)c1cc2oc(=O)c(C(=O)O)cc2s1] | 2-[(1-Benzyl-azetidine-2-carbonyl)- methyl-amino]-5-oxo-5H-thieno [3,2-b]pyran-6-carboxylic acid | NT |
| 56 | 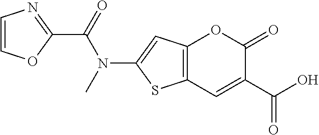 [SMILES: CN(C(=O)c1ncco1)c1cc2oc(=O)c(C(=O)O)cc2s1] | 2-[Methyl-(oxazole-2-carbonyl)- amino]-5-oxo-5H-thieno[3,2-b] pyran-6-carboxylic acid | NT |
| 57 | 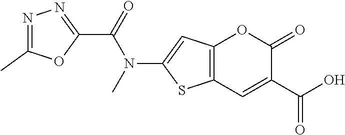 [SMILES: Cc1nnc(C(=O)N(C)c2cc3oc(=O)c(C(=O)O)cc3s2)o1] | 2-[Methyl-(5-methyl-[1,3,4] oxadiazole-2-carbonyl)-amino]-5- oxo-5H-thieno[3,2-b]pyran-6- carboxylic acid | NT |
| 58 | 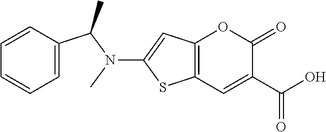 [SMILES: C[C@H](c1ccccc1)N(C)c1cc2oc(=O)c(C(=O)O)cc2s1] | 2-[Methyl-(1-phenyl-ethyl)-amino]- 5-oxo-5H-thieno[3,2-b]pyran-6- carboxylic acid | NT |
| 59 | 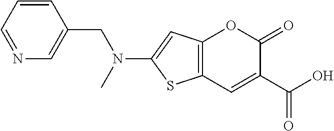 [SMILES: CN(Cc1cccnc1)c1cc2oc(=O)c(C(=O)O)cc2s1] | 2-(Methyl-pyridin-3-ylmethyl- amino)-5-oxo-5H-thieno[3,2-b] pyran-6-carboxylic acid | NT |
| 60 | 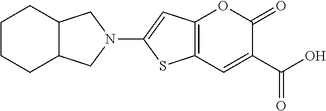 [SMILES: O=C(O)c1cc2sc(N3CC4CCCCC4C3)cc2oc1=O] | 2-(Octahydro-isoindol-2-yl)-5- oxo-5H-thieno[3,2-b]pyran-6- carboxylic acid | NT |
| 61 | 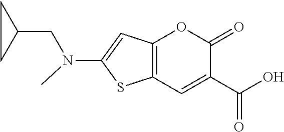 [SMILES: CN(CC1CC1)c1cc2oc(=O)c(C(=O)O)cc2s1] | 2-(Cyclopropylmethyl-methyl- amino)-5-oxo-5H-thieno[3,2-b] pyran-6-carboxylic acid | NT |
| 62 | 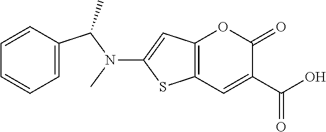 [SMILES: C[C@@H](c1ccccc1)N(C)c1cc2oc(=O)c(C(=O)O)cc2s1] | 2-[Methyl-(1-phenyl-ethyl)-amino]- 5-oxo-5H-thieno[3,2-b]pyran-6- carboxylic acid | NT |
| 63 | 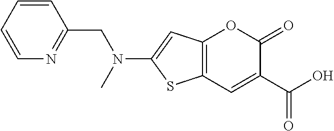 [SMILES: CN(Cc1ccccn1)c1cc2oc(=O)c(C(=O)O)cc2s1] | 2-(Methyl-pyridin-2-ylmethyl- amino)-5-oxo-5H-thieno[3,2- b]pyran-6-carboxylic acid | NT |
| 64 | 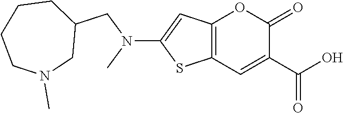 [SMILES: CN1CCCCC(CN(C)c2cc3oc(=O)c(C(=O)O)cc3s2)C1] | 2-[Methyl-(1-methyl-azepan-3- ylmethyl)-amino]-5-oxo-5H- thieno[3,2-b]pyran-6-carboxylic acid | NT |
| 65 | 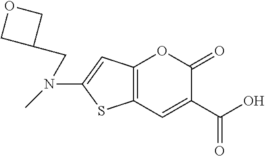 [SMILES: CN(CC1COC1)c1cc2oc(=O)c(C(=O)O)cc2s1] | 2-(Methyl-oxetan-3-ylmethyl- amino)-5-oxo-5H-thieno[3,2- b]pyran-6-carboxylic acid | NT |
| 66 | 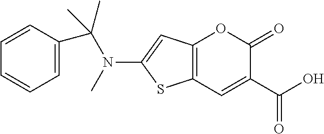 [SMILES: CN(c1cc2oc(=O)c(C(=O)O)cc2s1)C(C)(C)c1ccccc1] | 2-[Methyl-(1-methyl-1-phenyl- ethyl)-amino]-5-oxo-5H-thieno [3,2-b]pyran-6-carboxylic acid | NT |
| 67 | 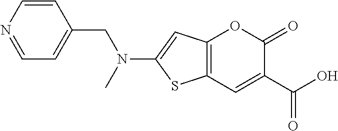 [SMILES: CN(Cc1ccncc1)c1cc2oc(=O)c(C(=O)O)cc2s1] | 2-(Methyl-pyridin-4-ylmethyl- amino)-5-oxo-5H-thieno[3,2-b] pyran-6-carboxylic acid | NT |
| 68 | 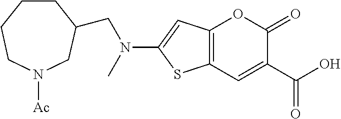 [SMILES: CC(=O)N1CCCCC(CN(C)c2cc3oc(=O)c(C(=O)O)cc3s2)C1] | 2-[(1-Acetyl-azepan-3-ylmethyl)- methyl-amino]-5-oxo-5H-thieno [3,2-b]pyran-6-carboxylic acid | NT |
| 69 | 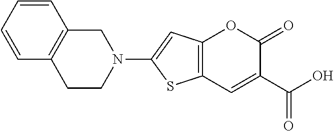 [SMILES: O=C(O)c1cc2sc(N3CCc4ccccc4C3)cc2oc1=O] | 2-(3,4-Dihydro-1H-isoquinolin- 2-yl)-5-oxo-5H-thieno[3,2-b] pyran-6-carboxylic acid | NT |
| 70 | 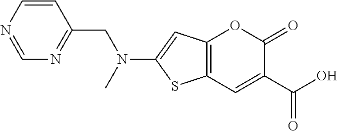 [SMILES: CN(Cc1ccncn1)c1cc2oc(=O)c(C(=O)O)cc2s1] | 2-(Methyl-pyrimidin-4-ylmethyl- amino)-5-oxo-5H-thieno[3,2-b] pyran-6-carboxylic acid | NT |
| 71 | 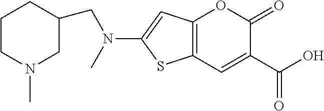 [SMILES: CN1CCCC(CN(C)c2cc3oc(=O)c(C(=O)O)cc3s2)C1] | 2-[Methyl-(1-methyl-piperidin- 3-ylmethyl)-amino]-5-oxo-5H- thieno[3,2-b]pyran-6-carboxylic acid | NT |
| 72 | 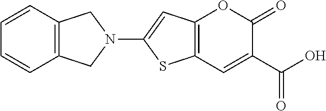 [SMILES: O=C(O)c1cc2sc(N3Cc4ccccc4C3)cc2oc1=O] | 2-(1,3-Dihydro-isoindol-2-yl)- 5-oxo-5H-thieno[3,2-b]pyran- 6-carboxylic acid | NT |
| 73 | 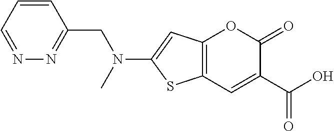 [SMILES: CN(Cc1cccnn1)c1cc2oc(=O)c(C(=O)O)cc2s1] | 2-(Methyl-pyridazin-3-ylmethyl- amino)-5-oxo-5H-thieno[3,2-b] pyran-6-carboxylic acid | NT |
| 74 | 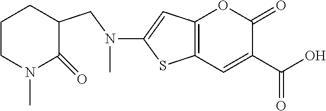 [SMILES: CN1CCCC(CN(C)c2cc3oc(=O)c(C(=O)O)cc3s2)C1=O] | 2-[Methyl-(1-methyl-2-oxo- piperidin-3-ylmethyl)-amino]-5- oxo-5H-thieno[3,2-b]pyran-6- carboxylic acid | NT |
| 75 | 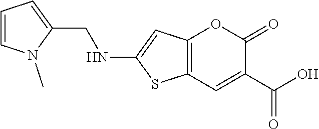 [SMILES: Cn1cccc1CNc1cc2oc(=O)c(C(=O)O)cc2s1] | 2-[(1-Methyl-1H-pyrrol-2- ylmethyl)-amino]-5-oxo-5H- thieno[3,2-b]pyran-6-carboxylic acid | NT |
| 76 | 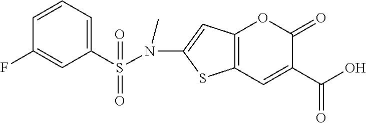 [SMILES: CN(c1cc2oc(=O)c(C(=O)O)cc2s1)S(=O)(=O)c1cccc(F)c1] | 2-[(3-Fluoro-benzenesulfonyl)- methyl-amino]-5-oxo-5H-thieno [3,2-b]pyran-6-carboxylic acid | NT |
| 77 | 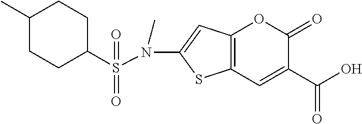 [SMILES: CC1CCC(S(=O)(=O)N(C)c2cc3oc(=O)c(C(=O)O)cc3s2)CC1] | 2-[Methyl-(4-methyl- cyclohexanesulfonyl)-amino]- 5-oxo-5H-thieno[3,2-b]pyran- 6-carboxylic acid | NT |
| 78 | 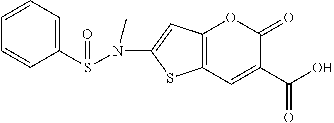 [SMILES: CN(c1cc2oc(=O)c(C(=O)O)cc2s1)S(=O)c1ccccc1] | 2-(Benzenesulfinyl-methyl-amino)- 5-oxo-5H-thieno[3,2-b]pyran-6- carboxylic acid | NT |
| 79 | 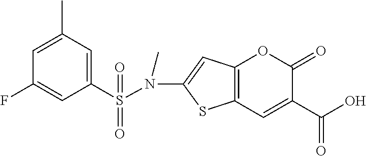 [SMILES: Cc1cc(F)cc(S(=O)(=O)N(C)c2cc3oc(=O)c(C(=O)O)cc3s2)c1] | 2-[(3-Fluoro-5-methyl- benzenesulfonyl)-methyl-amino]- 5-oxo-5H-thieno[3,2-b]pyran-6- carboxylic acid | NT |
| 80 | 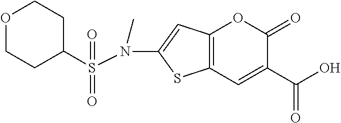 [SMILES: CN(c1cc2oc(=O)c(C(=O)O)cc2s1)S(=O)(=O)C1CCOCC1] | 2-[Methyl-(tetrahydro-pyran-4- sulfonyl)-amino]-5-oxo-5H-thieno [3,2-b]pyran-6-carboxylic acid | NT |
| 81 | 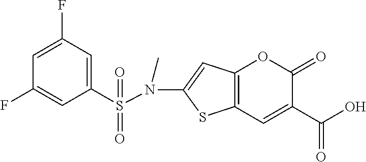 [SMILES: CN(c1cc2oc(=O)c(C(=O)O)cc2s1)S(=O)(=O)c1cc(F)cc(F)c1] | 2-[(3,5-Difluoro-benzenesulfonyl)- methyl-amino]-5-oxo-5H-thieno [3,2-b]pyran-6-carboxylic acid | NT |
| 82 | 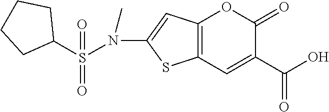 [SMILES: CN(c1cc2oc(=O)c(C(=O)O)cc2s1)S(=O)(=O)C1CCCC1] | 2-(Cyclopentanesulfonyl-methyl- amino)-5-oxo-5H-thieno[3,2-b] pyran-6-carboxylic acid | NT |
| 83 | 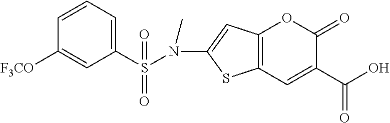 [SMILES: CN(c1cc2oc(=O)c(C(=O)O)cc2s1)S(=O)(=O)c1cccc(OC(F)(F)F)c1] | 2-[Methyl-(3-trifluoromethoxy- benzenesulfonyl)-amino]-5-oxo- 5H-thieno[3,2-b]pyran-6-carboxylic acid | NT |
| 84 | 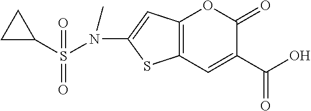 [SMILES: CN(c1cc2oc(=O)c(C(=O)O)cc2s1)S(=O)(=O)C1CC1] | 2-(Cyclopropanesulfonyl-methyl- amino)-5-oxo-5H-thieno[3,2-b] pyran-6-carboxylic acid | NT |
| 85 | 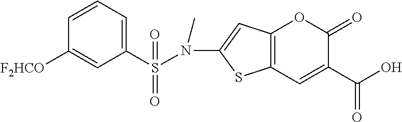 [SMILES: CN(c1cc2oc(=O)c(C(=O)O)cc2s1)S(=O)(=O)c1cccc(OC(F)F)c1] | 2-[(3-Difluoromethoxy- benzenesulfonyl)-methyl-amino]-5- oxo-5H-thieno[3,2-b]pyran-6- carboxylic acid | NT |
| 86 | 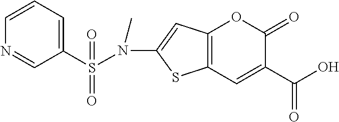 [SMILES: CN(c1cc2oc(=O)c(C(=O)O)cc2s1)S(=O)(=O)c1cccnc1] | 2-[Methyl-(pyridine-3-sulfonyl)- amino]-5-oxo-5H-thieno[3,2-b] pyran-6-carboxylic acid | NT |
| 87 | 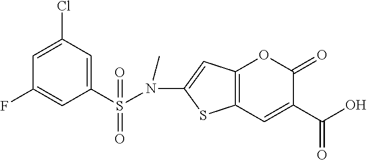 [SMILES: CN(c1cc2oc(=O)c(C(=O)O)cc2s1)S(=O)(=O)c1cc(F)cc(Cl)c1] | 2-[(3-Chloro-5-fluoro- benzenesulfonyl)-methyl-amino]- 5-oxo-5H-thieno[3,2-b]pyran-6- carboxylic acid | NT |
| 88 | 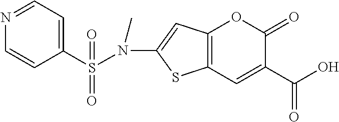 [SMILES: CN(c1cc2oc(=O)c(C(=O)O)cc2s1)S(=O)(=O)c1ccncc1] | 2-[Methyl-(pyridine-4-sulfonyl)- amino]-5-oxo-5H-thieno[3,2-b] pyran-6-carboxylic acid | NT |
| 89 | 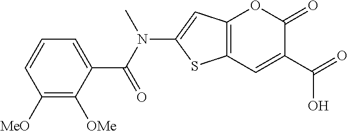 [SMILES: COc1cccc(C(=O)N(C)c2cc3oc(=O)c(C(=O)O)cc3s2)c1OC] | 2-(2,3-dimethoxy-N- methylbenzamido)-5-oxo-5H- thieno[3,2-b]pyran-6-carboxylic acid | NT |
| 90 | 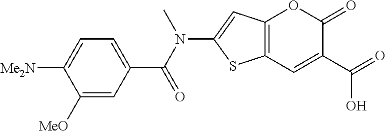 [SMILES: COc1cc(C(=O)N(C)c2cc3oc(=O)c(C(=O)O)cc3s2)ccc1N(C)C] | 2-(4-(dimethylamino)-3-methoxy-N- methylbenzamido)-5-oxo-5H-thieno [3,2-b]pyran-6-carboxylic acid | NT |
| 91 | 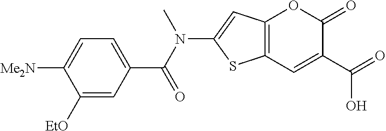 [SMILES: CCOc1cc(C(=O)N(C)c2cc3oc(=O)c(C(=O)O)cc3s2)ccc1N(C)C] | 2-(4-(dimethylamino)-3-ethoxy-N- methylbenzamido)-5-oxo-5H-thieno [3,2-b]pyran-6-carboxylic acid | NT |
| 92 | 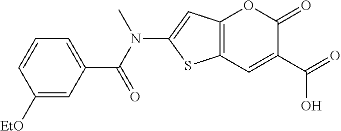 [SMILES: CCOc1cccc(C(=O)N(C)c2cc3oc(=O)c(C(=O)O)cc3s2)c1] | 2-(3-ethoxy-N-methylbenzamido)-5- oxo-5H-thieno[3,2-b]pyran-6- carboxylic acid | NT |
| 93 | 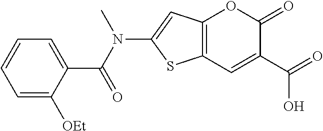 [SMILES: CCOc1ccccc1C(=O)N(C)c1cc2oc(=O)c(C(=O)O)cc2s1] | 2-(2-ethoxy-N-methylbenzamido)- 5-oxo-5H-thieno[3,2-b]pyran-6- carboxylic acid | NT |
| 94 | 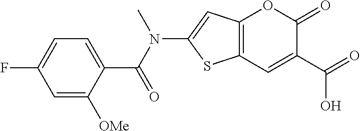 [SMILES: COc1cc(F)ccc1C(=O)N(C)c1cc2oc(=O)c(C(=O)O)cc2s1] | 2-(4-fluoro-2-methoxy-N- methylbenzamido)-5-oxo-5H- thieno[3,2-b]pyran-6-carboxylic acid | NT |
| 95 | 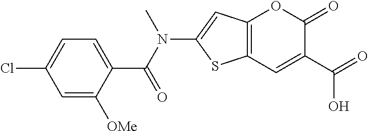 [SMILES: COc1cc(Cl)ccc1C(=O)N(C)c1cc2oc(=O)c(C(=O)O)cc2s1] | 2-(4-chloro-2-methoxy-N- methylbenzamido)-5-oxo-5H- thieno[3,2-b]pyran-6-carboxylic acid | NT |
| 96 | 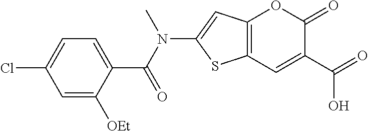 [SMILES: CCOc1cc(Cl)ccc1C(=O)N(C)c1cc2oc(=O)c(C(=O)O)cc2s1] | 2-(4-chloro-2-ethoxy-N- methylbenzamido)-5-oxo- 5H-thieno[3,2-b]pyran-6- carboxylic acid | NT |
| 97 | 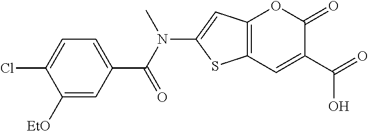 [SMILES: CCOc1cc(C(=O)N(C)c2cc3oc(=O)c(C(=O)O)cc3s2)ccc1Cl] | 2-(4-chloro-3-ethoxy-N- methylbenzamido)-5-oxo- 5H-thieno[3,2-b]pyran-6- carboxylic acid | NT |
| 98 | 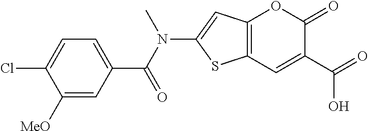 [SMILES: COc1cc(C(=O)N(C)c2cc3oc(=O)c(C(=O)O)cc3s2)ccc1Cl] | 2-(4-chloro-3-methoxy-N- methylbenzamido)-5-oxo- 5H-thieno[3,2-b]pyran-6- carboxylic acid | NT |
| 99 | 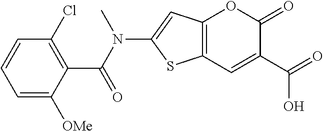 [SMILES: COc1cccc(Cl)c1C(=O)N(C)c1cc2oc(=O)c(C(=O)O)cc2s1] | 2-(2-chloro-6-methoxy-N- methylbenzamido)-5-oxo- 5H-thieno[3,2-b]pyran-6- carboxylic acid | NT |
| 100 | 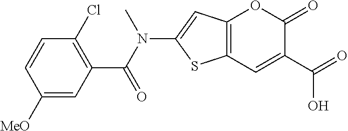 [SMILES: COc1ccc(Cl)c(C(=O)N(C)c2cc3oc(=O)c(C(=O)O)cc3s2)c1] | 2-(2-chloro-5-methoxy-N- methylbenzamido)-5-oxo-5H- thieno[3,2-b]pyran-6-carboxylic acid | NT |

IC₅₀:
A = <1 uM;
B = 1-10 uM;
C = >10;
NT = Not Tested

TABLE II

| | | Lactate Consumption [{Lactate |
|---|---|---|
| | | Kit or Lactate meter or YSI |
| | | bioanalyzer}(% |
| Example | Structure | inhibition)/(IC₅₀; uM)] |
| 1 | 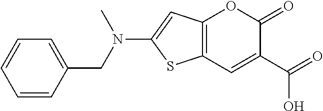 [SMILES: CN(Cc1ccccc1)c1cc2oc(=O)c(C(=O)O)cc2s1] | A |
| 2 | 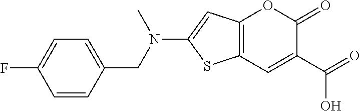 [SMILES: CN(Cc1ccc(F)cc1)c1cc2oc(=O)c(C(=O)O)cc2s1] | A |

MTS Cell Proliferation Assay

Cytotoxicity of the inhibition of monocarboxylate transporters of the invention was determined and shown in Table 1. The anti-proliferation effect of MCT inhibition was investigated across a panel of solid and haemotological tumor cell lines. Cells were routinely cultured in their appropriate growth medium. On day 1, between 5,000-20,000 cells/well were plated into 96-well plates. 100 μL of phosphate buffered saline solution was added to the external wells to prevent media evaporation. Plates were incubated in growth medium overnight at 37° C. in the presence of 5% CO₂. On day 2, dry weight compound stocks were dissolved to a concentration of 20 mM in 100% DMSO. Compounds were further diluted in the assay medium; 10 mM lactate medium (without glucose, pyruvate, and glutamine) or RPMI medium or appropriate medium to generate a final dose range of 10 nM to 100 μM. Growth medium in the 96-well plate was replaced with the assay medium (10 mM lactate medium or RPMI medium or appropriate medium), and compounds were added to each well in the plate at different concentrations via serial dilution or pre-prepared solutions in assay medium. Plates were then incubated at 37° C. in the presence of 5% CO₂ for a further 72 hours. On day 5, 20 μL of CellTiter 96 AQ MTS reagent was added to each well and the plate was returned to the incubator for 2 hours. In case of lactate medium, the medium was replaced by 100 μL of growth medium and 20 μL of CellTiter 96 AQ MTS reagent. MTS is bioreduced by NADPH or NADH produced by dehydrogenase enzymes in metabolically active cells into a coloured formazan product that is soluble in tissue culture medium. The amount of coloured formazan product is directly proportional to the number of living cells in culture. The absorbance of the plates was read on a Synergy H4 plate reader using 490 nM measurement wavelength. Dose response curves were plotted and IC₅₀ values were calculated using GraphPrism. The IC₅₀ value is equivalent to the concentration of compound that causes 50% inhibition of growth calculated from the compound treated signal to the vehicle treated signal.

MTT Cell Proliferation Assay

The MIT assay is a colorimetric assay for assessing cell viability similar to MTS assay. NAD(P)H-dependent cellular oxidoreductase enzymes may, under defined conditions, reflect the number of viable cells present. These enzymes are capable of reducing the tetrazolium dye MTT 3-(4,5-dimethylthiazol-2-yl)-2,5-diphenyltetrazolium bromide to its insoluble formazan, which has a purple color. MTT powder is dissolved in Dulbecco's Phosphate Buffered Saline, pH=7.4 (DPBS) to 5 mg/ml as stock solution. Cells and test compounds in 96-well plates were prepared containing a final volume of 100 μL/well as described in the MTS assay. The assay was performed similar to MTS assay above. After 72 hours incubation, 10 μL MTT solution per well were added to achieve a final concentration of 0.45 mg/mL, and then incubated for 4 hours at 37° C. The medium was removed and plates were air dried for 10 minutes at dark. Then 100 μL of DMSO was added to each well and incubated at dark for 30 minutes with mild shaking. The absorbance was read at 570 nm.

Lactate Consumption Assay in Tumor Cell Lines.

The inhibition of monocarboxylate transporters of the invention was determined and data are shown in Table II. Cells are maintained in their appropriate growth medium (DMEM medium with 4.5 g/L glucose, 4 mM L-glutamine supplemented with 10% FBS and P/S (growth medium). 500,000 cells/well were seeded in 24-well plate in growth medium for 6 hours. Replace the growth medium with 1 mL lactate medium (10 mM lactate in base DMEM without sodium pyruvate) for overnight. Cells were treated with compounds in 1 mL lactate medium for 24 hours. The culture medium was collected and centrifuged at 12,000 rpm for 5 minutes at 4° C. to get rid of any cell debris. An aliquot of 0.5 mL of the supernatant was loaded to a deproteinizing column, centrifuged at 12,000 rpm for 15 minutes at 4° C. The flow-through was collected and stored at −80° C. for future analysis. The amount of lactate in the supernatant was analyzed by enzymatic L-Lactate Kit II (Eton Bioscience Inc.) or commercially available YSI 2900 bioanalyzer according to manufacturer's instructions. Briefly, 50 μL of 10 times diluted sample was mixed with 50 μL reaction mixture. Lactic acid is oxidized by enzyme reactions to yield color product, which can be measured in dual modes, either at 570 nm for colorimetric assay or with Ex 530-560/Ern 570-595 nm fluorescence assay. And the color or fluorescence intensity is proportional to lactic acid concentration, and therefore the sample lactic acid concentration can be accurately calculated based on the lactic acid standards. The signal was read on a Synergy H4 plate reader using 570 nM measurement wavelength, and the lactate consumption was calculated by medium lactate concentration at start point (10 mM) subtracted the end point.

Tumor Xenograft Model.

In order to test the in vivo efficacy of the invention, Example 9 was tested according to the protocol of Iorns to al. [Iorns E, Drews-Elger K, Ward T M, Dean S, Clarke J, Berry D, et al. (2012) A New Mouse Model for the Study of Human Breast Cancer Metastasis. PLoS ONE 7(10): e47995. https://doi.org/10.1371/journal.pone.0047995] Twenty-eight female NOD-SCID mice (Charles River) were injected orthotopically with basal MDA-MB-231 human breast carcinoma cells into the mammary fat pads. Mice were monitored for development of primary xenograft tumors, and dosing with test compound began when mice exhibited a median tumor volume of 125 mm³. Mice were dosed qd with 30 mg/kg and 75 mg/kg p.o. of Example 9. At nine days after start of treatment tumor volume was roughly 50% of control in mice dosed at 30 mg/kg and roughly 30% of control in mice dosed at 75 mg/kg.

We claim:

1. A compound of formula I: 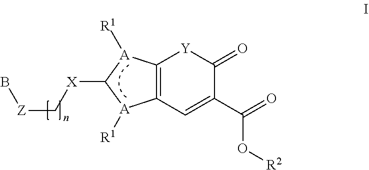
wherein:
n is 0, 1, or 2;
X is O or NR″;
Y is O or NR″;
Z is a bond, CH₂, C═O, SO₂; 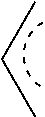
is 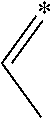 [SMILES: *=CC]
or 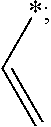
A is chosen independently in each occurrence from N, NR″, S, O, CR″ and CHR″;
R¹ is selected from the group consisting of hydrogen, halogen, alkyl, —CHF₂, —CF₃, —CN, —C(O)R″, —C(O)OR″, —SO₂R″, —C(O)NR″₂, —C(O)N(OR″)R″ and 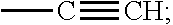
R² is selected from the group consisting of
Hydrogen;
—C(O)R″;
—(CH₂)₀₋₄C(O)R″;
—(CH₂)₀₋₄C(O)OR″;
optionally substituted C₁₋₆ alkyl;
optionally substituted 3-8 membered saturated or partially unsaturated cycloalkyl ring;
optionally substituted 3-8 membered saturated or partially unsaturated heterocycloalkyl ring having 1-2 heteroatoms independently selected from nitrogen, oxygen, and sulfur;
optionally substituted phenyl; and
optionally substituted 5-6 membered heteroaryl ring having 1-4 heteroatoms independently selected from nitrogen, oxygen, and sulfur;
B is a ring selected from
a 3-8 membered saturated or partially unsaturated monocyclic carbocyclic ring, phenyl,
an 8-10 membered bicyclic aryl ring,
a 3-8 membered saturated or partially unsaturated monocyclic or bicyclic heterocyclic ring having 1-2 heteroatoms independently selected from nitrogen, oxygen, and sulfur,
a 5-6 membered monocyclic heteroaryl ring having 1-4 heteroatoms independently selected from nitrogen, oxygen, and sulfur, and
an 8-10 membered bicyclic heteroaryl ring having 1-4 heteroatoms independently selected from nitrogen, oxygen, and sulfur,
wherein B is optionally substituted with one or more R″ substituents;
R″ is chosen from
R¹;
3-8 membered saturated or partially unsaturated cycloalkyl ring, optionally substituted with halogen or C₁₋₆ alkyl;
3-8 membered saturated or partially unsaturated heterocycloalkyl ring having 1-2 heteroatoms independently selected from nitrogen, oxygen, and sulfur, said ring optionally substituted with halogen or C₁₋₆ alkyl;
phenyl optionally substituted with halogen or C₁₋₆ alkyl; and
5-6 membered heteroaryl ring having 1-4 heteroatoms independently selected from nitrogen, oxygen, and sulfur, said ring optionally substituted with halogen or C₁₋₆ alkyl.

2. A compound according to claim 1, wherein the compound is of the formula II:

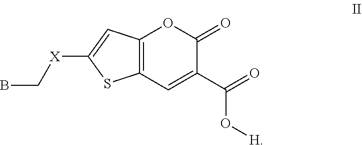

3. A compound according to claim 1, wherein the compound is of the formula III:

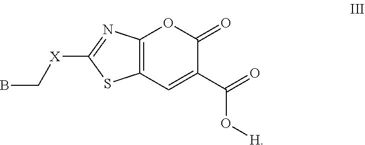

4. A compound according to claim 2, wherein, B is selected from:

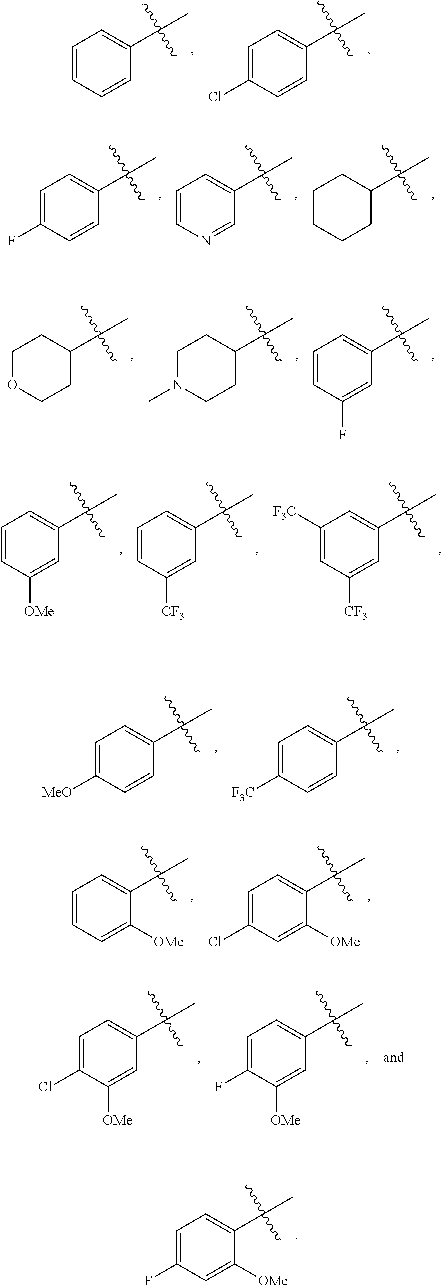

5. A compound according to claim 3, wherein, B is selected from:

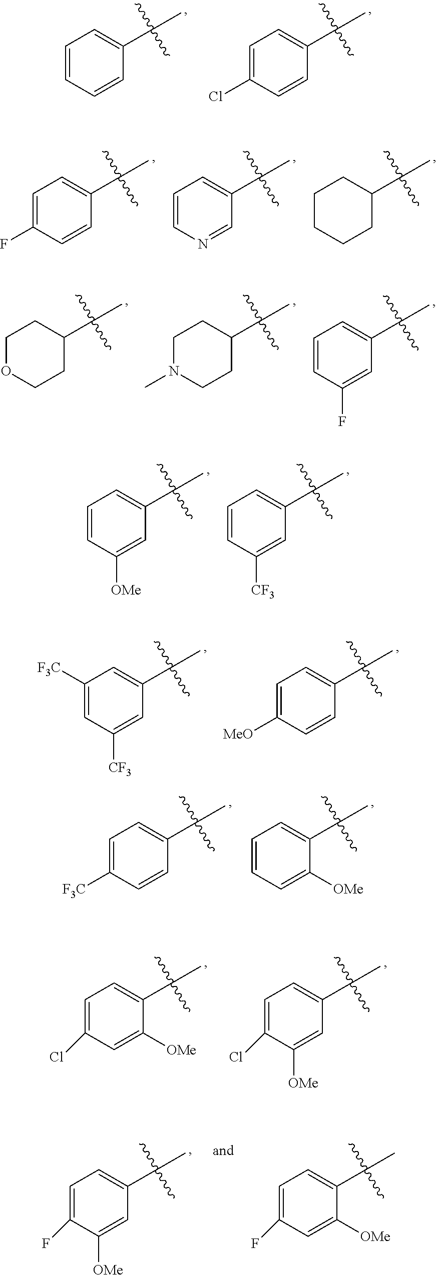

6. A compound according to claim 4, wherein, X is O, —NH or —NMe.

7. A compound according to claim 5, wherein, X is O, —NH, or —NCH₃.

8. A compound according to claim 1, wherein the compound is of the formula IV:

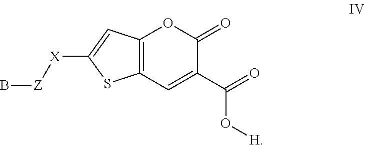

9. A compound according to claim 8, wherein the compound is of the formula V or VI:

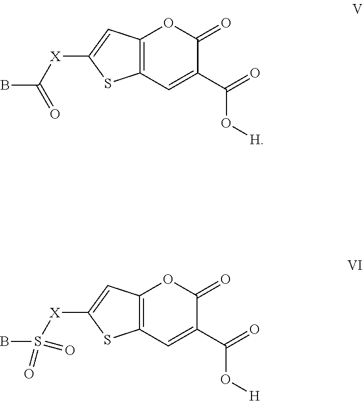

10. A compound according to claim 9, wherein, B is selected from:

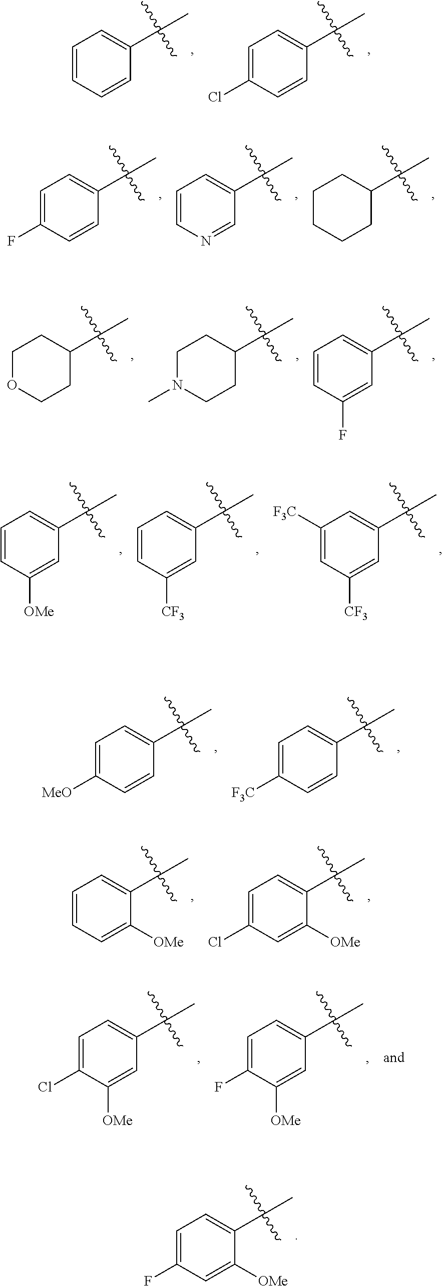

11. A compound according to claim 10, wherein, X is O, —NH or —NMe.

12. A compound according to claim 1, wherein the compound is of the formula VI:

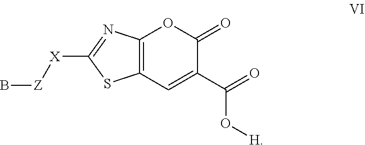

13. A compound according to claim 12, wherein the compound is of the formula VII:

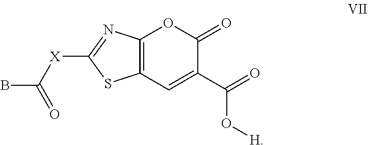

14. A compound according to claim 13, wherein, B is selected from:

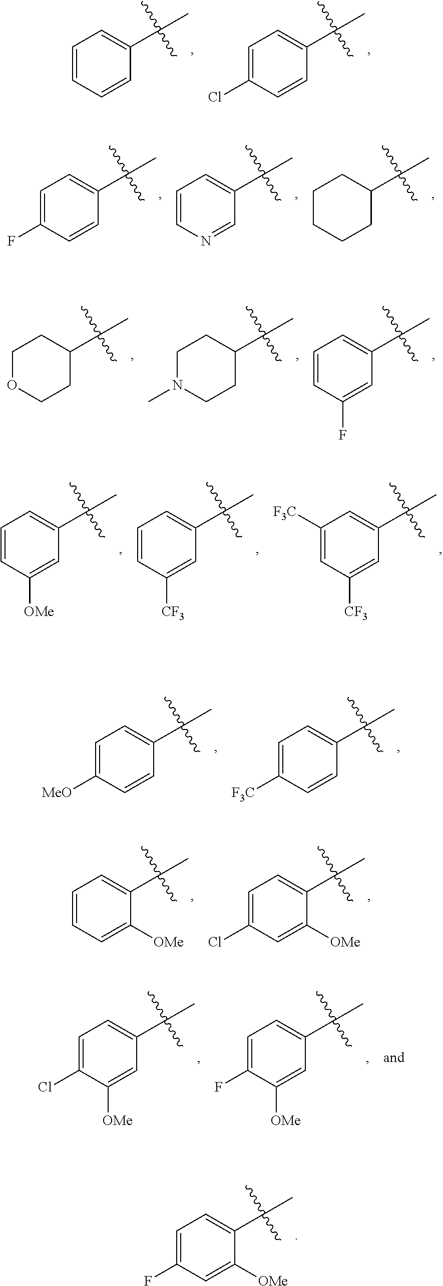

15. A compound according to claim 14, wherein, X is O, —NH or —NMe.

16. A compound according to claim 1 selected from:

| Structure | Name |
|---|---|
| 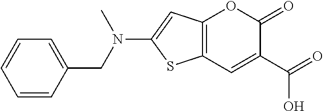 [SMILES: CN(Cc1ccccc1)c1cc2oc(=O)c(C(=O)O)cc2s1] | 2-(benzyl(methyl)amino)-5-oxo-5H-thieno[3,2- b]pyran-6-carboxylic acid |
| 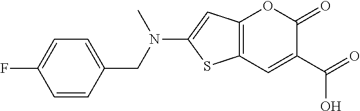 [SMILES: CN(Cc1ccc(F)cc1)c1cc2oc(=O)c(C(=O)O)cc2s1] | 2-((4-fluorobenzyl)(methyl)amino)-5-oxo-5H- thieno[3,2-b]pyran-6-carboxylic acid |
| 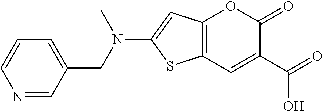 [SMILES: CN(Cc1cccnc1)c1cc2oc(=O)c(C(=O)O)cc2s1] | 2-(methyl(pyridin-3-ylmethyl)amino)-5-oxo-5H- thieno[3,2-b]pyran-6-carboxylic acid |
| 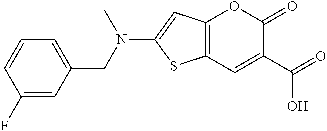 [SMILES: CN(Cc1cccc(F)c1)c1cc2oc(=O)c(C(=O)O)cc2s1] | 2-((3-fluorobenzyl)(methyl)amino)-5-oxo-5H- thieno[3,2-b]pyran-6-carboxylic acid |
| 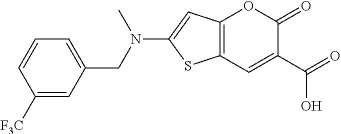 [SMILES: CN(Cc1cccc(C(F)(F)F)c1)c1cc2oc(=O)c(C(=O)O)cc2s1] | 2-(methyl(3-(trifluoromethyl)benzyl)amino)-5- oxo-5H-thieno[3,2-b]pyran-6-carboxylic acid |
| 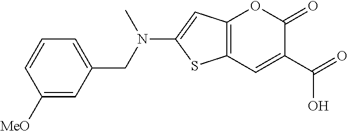 [SMILES: COc1cccc(CN(C)c2cc3oc(=O)c(C(=O)O)cc3s2)c1] | 2-((3-methoxpenzyl)(methyl)amino)-5-oxo-5H- thieno[3,2-b]pyran-6-carboxylic acid |
| 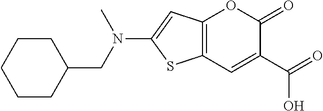 [SMILES: CN(CC1CCCCC1)c1cc2oc(=O)c(C(=O)O)cc2s1] | 2-((cyclohexylmethyl)(methyl)amino)-5-oxo-5H- thieno[3,2-b]pyran-6-carboxylic acid |
| 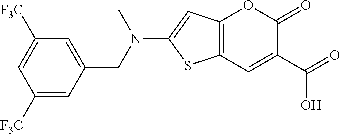 [SMILES: CN(Cc1cc(C(F)(F)F)cc(C(F)(F)F)c1)c1cc2oc(=O)c(C(=O)O)cc2s1] | 2-((3,5-bis(trifluoromethypenzyl)(methyl)amino)-5-oxo- 5H-thieno[3,2-b]pyran-6-carboxylic acid |
| 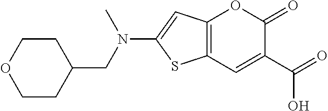 [SMILES: CN(CC1CCOCC1)c1cc2oc(=O)c(C(=O)O)cc2s1] | 2-(methyl((tetrahydro-2H-pyran-4- yl)methyl)amino)-5-oxo-5H-thieno[3,2-b]pyran-6- carboxylic acid |
| 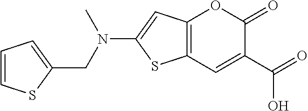 [SMILES: CN(Cc1cccs1)c1cc2oc(=O)c(C(=O)O)cc2s1] | 2-(methyl(thiophen-2-ylmethyl)amino)-5-oxo-5H- thieno[3,2-b]pyran-6-carboxylic acid |
| 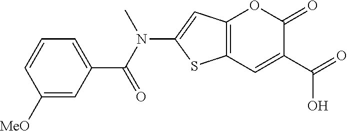 [SMILES: COc1cccc(C(=O)N(C)c2cc3oc(=O)c(C(=O)O)cc3s2)c1] | 2-(3-methoxy-N-methylbenzamido)-5-oxo-5H- thieno[3,2-b]pyran-6-carboxylic acid |
| 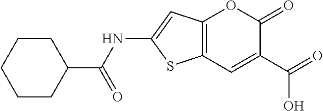 [SMILES: O=C(O)c1cc2sc(NC(=O)C3CCCCC3)cc2oc1=O] | 2-(cyclohexanecarboxamido)-5-oxo-5H- thieno[3,2-b]pyran-6-carboxylic acid |
| 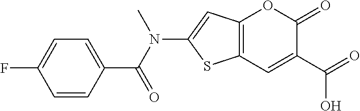 [SMILES: CN(C(=O)c1ccc(F)cc1)c1cc2oc(=O)c(C(=O)O)cc2s1] | 2-(4-fluoro-N-methylbenzamido)-5-oxo-5H- thieno[3,2-b]pyran-6-carboxylic acid |
| 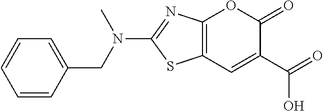 [SMILES: CN(Cc1ccccc1)c1nc2oc(=O)c(C(=O)O)cc2s1] | 2-(benzyl(methyl)amino)-5-oxo-5H-pyrano[2,3- d]thiazole-6-carboxylic acid |
| 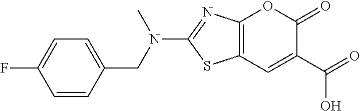 [SMILES: CN(Cc1ccc(F)cc1)c1nc2oc(=O)c(C(=O)O)cc2s1] | 2-((4-fluorobenzyl)(methyl)amino)-5-oxo-5H- pyrano[2,3-d]thiazole-6-carboxylic acid |
| 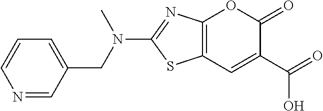 [SMILES: CN(Cc1cccnc1)c1nc2oc(=O)c(C(=O)O)cc2s1] | 2-(methyl(pyridin-3-ylmethyl)amino)-5-oxo-5H- pyrano[2,3-d]thiazole-6-carboxylic acid |
| 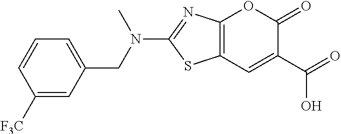 [SMILES: CN(Cc1cccc(C(F)(F)F)c1)c1nc2oc(=O)c(C(=O)O)cc2s1] | 2-(methyl(3-(trifluoromethyl)benzyl)amino)-5- oxo-5H-pyrano[2,3-d]thiazole-6-carboxylic acid |
| 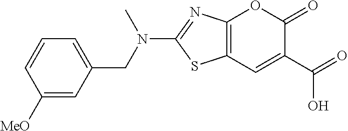 [SMILES: COc1cccc(CN(C)c2nc3oc(=O)c(C(=O)O)cc3s2)c1] | 2-((3-methoxpenzyl)(methyl)amino)-5-oxo-5H- pyrano[2,3-d]thiazole-6-carboxylic acid |
| 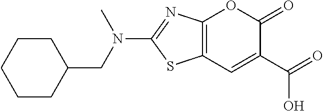 [SMILES: CN(CC1CCCCC1)c1nc2oc(=O)c(C(=O)O)cc2s1] | 2-((cyclohexylmethyl)(methyl)amino)-5-oxo-5H- pyrano[2,3-d]thiazole-6-carboxylic acid |
| 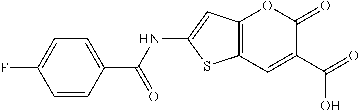 [SMILES: O=C(Nc1cc2oc(=O)c(C(=O)O)cc2s1)c1ccc(F)cc1] | 2-(4-fluorobenzamido)-5-oxo-5H-thieno[3,2- b]pyran-6-carboxylic acid |
| 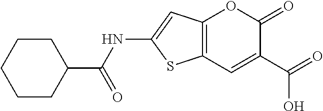 [SMILES: O=C(O)c1cc2sc(NC(=O)C3CCCCC3)cc2oc1=O] | 2-(cyclohexanecarboxamido)-5-oxo-5H- thieno[3,2-b]pyran-6-carboxylic acid |
| 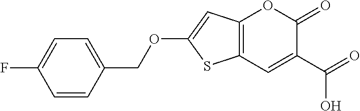 [SMILES: O=C(O)c1cc2sc(OCc3ccc(F)cc3)cc2oc1=O] | 2-((4-fluorobenzyl)oxy)-5-oxo-5H-thieno[3,2- b]pyran-6-carboxylic acid |
| 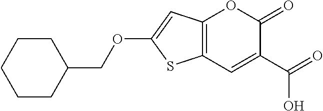 [SMILES: O=C(O)c1cc2sc(OCC3CCCCC3)cc2oc1=O] | 2-(cyclohexylmethoxy)-5-oxo-5H-thieno[3,2- b]pyran-6-carboxylic acid |
| 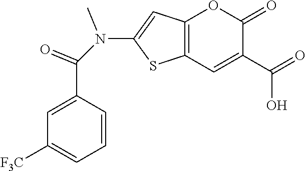 [SMILES: CN(C(=O)c1cccc(C(F)(F)F)c1)c1cc2oc(=O)c(C(=O)O)cc2s1] | 2-[Methyl-(3-trifluoromethyl-benzoyl)-amino]-5- oxo-5H-thieno[3,2-b]pyran-6-carboxylic acid |
| 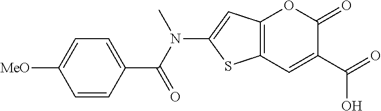 [SMILES: COc1ccc(C(=O)N(C)c2cc3oc(=O)c(C(=O)O)cc3s2)cc1] | 2-[(4-Methoxy-benzoyl)-methyl-amino]-5-oxo- 5H-thieno[3,2-b]pyran-6-carboxylic acid |
| 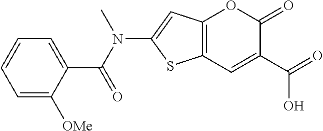 [SMILES: COc1ccccc1C(=O)N(C)c1cc2oc(=O)c(C(=O)O)cc2s1] | 2-[(2-Methoxy-benzoyl)-methyl-amino]-5-oxo- 5H-thieno[3,2-b]pyran-6-carboxylic acid |
| 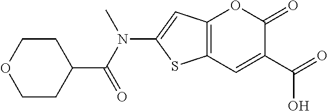 [SMILES: CN(C(=O)C1CCOCC1)c1cc2oc(=O)c(C(=O)O)cc2s1] | 2-[Methyl-(tetrahydro-pyran-4-carbonyl)-amino]- 5-oxo-5H-thieno[3,2-b]pyran-6-carboxylic acid |
| 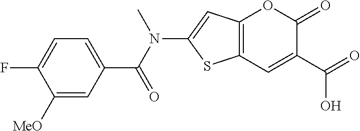 [SMILES: COc1cc(C(=O)N(C)c2cc3oc(=O)c(C(=O)O)cc3s2)ccc1F] | 2-[(4-Fluoro-3-methoxy-benzoyl)-methyl-amino]- 5-oxo-5H-thieno[3,2-b]pyran-6-carboxylic acid |
| 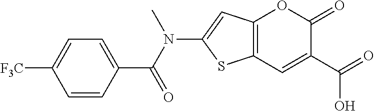 [SMILES: CN(C(=O)c1ccc(C(F)(F)F)cc1)c1cc2oc(=O)c(C(=O)O)cc2s1] | 2-[Methyl-(4-trifluoromethyl-benzoyl)-amino]-5- oxo-5H-thieno[3,2-b]pyran-6-carboxylic acid |
| 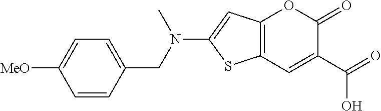 [SMILES: COc1ccc(CN(C)c2cc3oc(=O)c(C(=O)O)cc3s2)cc1] | 2-[(4-Methoxy-benzyl)-methyl-amino]-5-oxo-5H- thieno[3,2-b]pyran-6-carboxylic acid |
| 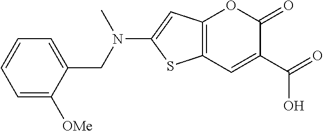 [SMILES: COc1ccccc1CN(C)c1cc2oc(=O)c(C(=O)O)cc2s1] | 2-[(2-Methoxy-benzyl)-methyl-amino]-5-oxo-5H- thieno[3,2-b]pyran-6-carboxylic acid |
| 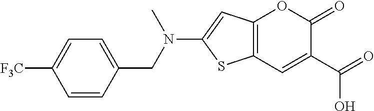 [SMILES: CN(Cc1ccc(C(F)(F)F)cc1)c1cc2oc(=O)c(C(=O)O)cc2s1] | 2-[Methyl-(4-trifluoromethyl-benzyl)-amino]-5- oxo-5H-thieno[3,2-b]pyran-6-carboxylic acid |
| 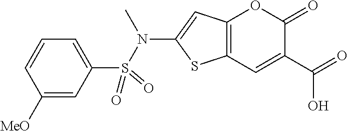 [SMILES: COc1cccc(S(=O)(=O)N(C)c2cc3oc(=O)c(C(=O)O)cc3s2)c1] | 2-[(3-Methoxy-benzenesulfonyl)-methyl-amino]- 5-oxo-5H-thieno[3,2-b]pyran-6-carboxylic acid |
| 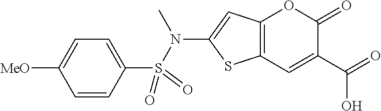 [SMILES: COc1ccc(S(=O)(=O)N(C)c2cc3oc(=O)c(C(=O)O)cc3s2)cc1] | 2-[(4-Methoxy-benzenesulfonyl)-methyl-amino]- 5-oxo-5H-thieno[3,2-b]pyran-6-carboxylic acid |
| 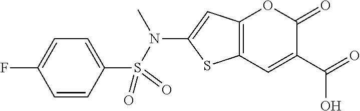 [SMILES: CN(c1cc2oc(=O)c(C(=O)O)cc2s1)S(=O)(=O)c1ccc(F)cc1] | 2-[(4-Fluoro-benzenesulfonyl)-methyl-amino]-5- oxo-5H-thieno[3,2-b]pyran-6-carboxylic acid |
| 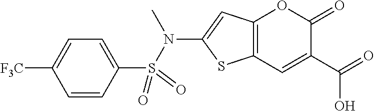 [SMILES: CN(c1cc2oc(=O)c(C(=O)O)cc2s1)S(=O)(=O)c1ccc(C(F)(F)F)cc1] | 2-[Methyl-(4-trifluoromethyl-benzenesulfonyl)- amino]-5-oxo-5H-thieno[3,2-b]pyran-6-carboxylic acid |
| 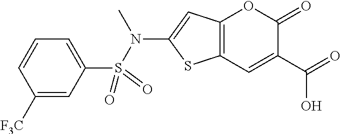 [SMILES: CN(c1cc2oc(=O)c(C(=O)O)cc2s1)S(=O)(=O)c1cccc(C(F)(F)F)c1] | 2-[Methyl-(3-trifluoromethyl-benzenesulfonyl)- amino]-5-oxo-5H-thieno[3,2-b]pyran-6-carboxylic acid. |

17. A method for modulating monocarboxylate transport comprising contacting a monocarboxylate transport protein with a therapeutically effective amount of a compound according to any of claims 1-16.

18. A method for treating a disorder associated with monocarboxylate transport comprising administering a therapeutically effective amount of a compound according to any of claims 1-16.

19. A method according to claim 18, wherein the said disorder is chosen from cancer, neoplastic disorders, disorders of abnormal tissue growth, and tissue and organ rejection.

20. A method according to claim 19 wherein said cancer is breast cancer.

21. A process for preparing a compound of formula I according to claim 1 of formula: 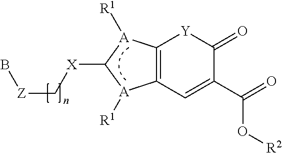
wherein X is NH and R² is CH₃, which comprises reacting an aldehyde of formula: 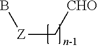
with an amine of formula 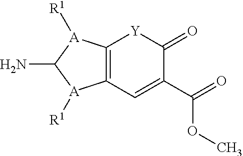
in the presence of a reducing agent.
